(19) United States
(12) Patent Application Publication
(10) Pub. No.: US 2026/0232946 A1
(43) Pub. Date: Aug. 13, 2026

(54) NITRIC OXIDE GENERATION, DILUTION, AND TOPICAL APPLICATIONS, RAPID, PRECISE, NITRIC OXIDE ANALYSIS AND TITRATION APPARATUS, COMPOSITIONS, SYSTEMS, AND METHODS

(71) Applicant: SYK Technologies LLC, Provo, UT (US)

(72) Inventors: J.W. Randolph Miller, Orem, UT (US); Ross M. Wille, Orem, UT (US); John J. Miller, Orem, UT (US); Stephen H. Fairbanks, Orem, UT (US)

(21) Appl. No.: 19/257,949

(22) Filed: Jul. 2, 2025

Related U.S. Application Data
(63) Continuation of application No. 17/727,651, filed on Apr. 22, 2022, now abandoned.
Application No. 17/727,651 is a continuation of application No. 14/810,303, filed on Jul. 27, 2015, now abandoned.
Application No. 14/810,303 is a continuation-in-part of application No. 14/194,977, filed on Mar. 3, 2014, now Pat. No. 9,302,238.
Application No. 14/194,977 is a continuation of application No. 13/197,695, filed on Aug. 3, 2011, now Pat. No. 8,685,467.
(60) Provisional application No. 61/370,214, filed on Aug. 3, 2010.
(60) Provisional application No. 62/138,856, filed on Mar. 26, 2015.

(51) Int. Cl. A61M 16/14; A61K 31/095; A61K 31/191; A61K 33/00; A61K 47/02; A61K 47/38; A61M 16/00; A61M 16/20

(52) U.S. Cl. CPC A61M 16/14; A61K 31/095; A61K 31/191; A61K 33/00; A61K 47/02; A61K 47/38; A61M 16/0003; A61M 16/0057; A61M 16/208; A61M 2202/0275; A61M 2205/3334; A61M 2205/50

(57) ABSTRACT

Topical applications that provide a nitric oxide therapy to a surface are provided. Systems for providing a topical nitric oxide therapy can comprise a nitrite medium in a first container, the nitrite medium comprising about 3% of a nitrite component by weight. The system comprises an acidic medium in a second container, the acidic medium comprising about 9% by weight of one or more acidic reactants. The nitrite medium and the acidic medium are configured to be combined to form a nitric oxide topical medium producing nitric oxide suitable for topical application and suitable for administering nitric oxide therapy wherein a therapeutically effective amount of the nitric oxide topical medium is applied to a treatment surface suitable for receiving nitric oxide therapy, whereby the application of the therapeutically effective amount is adapted to deliver a dose of nitric oxide at the treatment surface of a patient.

### RELATED APPLICATIONS

This application is a continuation of U.S. patent application Ser. No. 14/810,303, filed Jul. 27, 2015 and entitled RAPID, PRECISE, NITRIC OXIDE ANALYSIS AND TITRATION APPARATUS AND METHOD, which is a continuation-in-part of U.S. patent application Ser. No. 14/194,977, filed Mar. 3, 2014, entitled NITRIC OXIDE GENERATION, DILUTION, AND TOPICAL APPLICATION APPARATUS AND METHOD, issued as U.S. Pat. No. 9,302,238 on Apr. 5, 2016, which is a continuation of U.S. patent application Ser. No. 13/197,695, filed Aug. 3, 2011, entitled NITRIC OXIDE GENERATION, DILUTION, AND TOPICAL APPLICATION APPARATUS AND METHOD, issued as U.S. Pat. No. 8,685,467 on Apr. 1, 2014, and claims the benefit of U.S. Provisional Patent Application No. 61/370,214, filed Aug. 3, 2010, entitled NITRIC OXIDE GENERATOR AND DILUTION APPARATUS AND METHOD, and said U.S. patent application Ser. No. 14/810,303, filed Jul. 27, 2015 and entitled RAPID, PRECISE, NITRIC OXIDE ANALYSIS AND TITRATION APPARATUS AND METHOD claims the benefit of U.S. Provisional Patent Application Ser. No. 62/138,856, filed Mar. 26, 2015, entitled RAPID, PRECISE, NITRIC OXIDE ANALYSIS AND TITRATION APPARATUS AND METHOD; all of which are hereby incorporated by reference in their entirety.

### BACKGROUND

### The Field of the Invention

This invention relates generally to chemical reactors, and more specifically to apparatus and methods for generating nitric oxide. Still other applications may involve topical preparations introducing nitric oxide. This invention relates generally to measurement and control, and, more specifically, to apparatus and methods for analyzing and controlling delivery of nitric oxide over a comparatively wide range of dosage rates.

### BACKGROUND

The discovery of certain nitric oxide effects in live tissue garnered a Nobel prize. Much of the work in determining the mechanisms for implementing, and the effects of, nitric oxide administration are reported in literature. In its application however, introduction of nitric oxide to the human body has traditionally been extremely expensive. The therapies, compositions, preparations, hardware, and controls are sufficiently complex, large, and expensive to inhibit more widespread use of such therapies.

### BRIEF SUMMARY OF THE INVENTION

What is needed is a comparatively simple, easily controlled, and consequently inexpensive mechanism for introducing nitric oxide in a variable concentration. Also, needed is a simple introduction method for providing nitric oxide suitable for inhaling. Also, needed is a simple method for topical application of a nitric oxide therapy. User control precisely and responsively over a broader range from well below 100 parts per million (ppm) (even down to 10 ppm), in the infant dosing range, up to about 600 ppm for adult dosing, and over 1000 ppm for topical and other applications control and administration would be a great benefit from simplicity and reduction in size.

It would be an advance in the art to provide a generator suitable for administration of nitric oxide gas at variable concentrations. It would be an advance in the art to provide not only an independence from bottled gas, but independence from auxiliary power required for heat, pumping, instrumentation, controls, and the like. It would be an advance in the art to provide a medium and method for topical administration of nitric oxide gas. It would be an advance in the art to provide the antimicrobial, therapeutic, and analgesic benefits of nitric oxide through a topical application. It would be an advance in the art to provide a system suitable for administration of nitric oxide gas at precise, stable, yet variable concentrations whether or not from bottled gas.

In accordance with the foregoing, certain embodiments of apparatus and methods in accordance with the invention provide a reactor system that produces nitric oxide and regulates the flow and concentration of nitric oxide delivered. Nitric oxide may thus be introduced into the breathing air of a subject in a controlled manner. Nitric oxide amounts may be engineered to deliver a therapeutically effective amount on the order of single digits to the comparatively low hundreds (e.g., 100-500) of parts per million, or up to thousands of parts per million.

For example, sufficient nitric oxide may be presented through nasal inhalation to provide approximately five thousand parts per million in breathing air. This may be diluted due to additional bypass breathing, through nasal inhalation, or through oral inhalation.

One embodiment of an apparatus and method in accordance with the present invention may rely on a small reactor and a system of filters and pumps configured to provide a constant, regulated flow of nitric oxide. Other embodiments may provide an automated feedback system that monitors, controls, and adjusts the concentration of nitric oxide delivered.

Reactive compounds may be appropriately combined dry or in liquid form. Reactants may include potassium nitrite, sodium nitrite or the like. The reaction may begin upon introduction of heat. Heat may be initiated by liquid transport material to support ionic or other chemical reaction in a heat device.

An apparatus and method in accordance with the invention may include an insulating structure, shaped in a convenient, compact, efficient configuration such as a rectangular box, a cylindrical container, or the like. The insulating container may be sealed either inside or out with a containment vessel to prevent leakage of liquids therefrom. Such a system may not need to be constructed to sustain nor contain pressure. However, in certain embodiments, the reactor may need to be constructed to sustain and contain pressure.

In certain embodiments, chemical heaters may include metals finely divided to readily react with oxygen or solid oxidizers. Inside the containment vessel may be positioned heating elements such as those commercially available as chemical heaters. Various other chemical compositions of modest reactivity may be used to generate heat readily without the need for a flame, electrical power, or the like.

Above the heating element or heater within the containment vessel may be located a reactor. The reactor may preferably contain a chemically stable composition for generating nitric oxide. Such compositions, along with their formulation techniques, shapes, processes, and the like are disclosed in U.S. patent application Ser. No. 11/751,523, U.S. patent application Ser. No. 12/361,123, U.S. patent application Ser. No. 12/361,151, U.S. patent application Ser. No. 12/410,442, U.S. patent application Ser. No. 12/419,123, and U.S. Pat. No. 7,220,393, all incorporated herein by reference in their entireties as to all that they teach.

The reactor may include any composition suitable for generating nitric oxide by the activation available from heat. The reactor may be substantially sealed except for an inlet, such as a tubular member secured thereto to seal a path for entry of filtered air into the reactor, and an outlet, such as a tubular member secured thereto to seal a path for exit of nitric oxide from the reactor. The reactor may also include a structure to dissipate heat away from the reaction and facilitate the complete use of the reactants in the reactor.

In certain embodiments, a system of filters and pumps introduces air into the reactor and then conducts a controlled flow of nitric oxide out of the reactor. Accordingly, a system may include filters and pumps to introduce air into the reactor, control production of nitric oxide in the reactor, and conduct nitric oxide out of the reactor. The system may include devices controlling the pumps and the flow of nitric oxide.

Ultimately, an apparatus in accordance with the invention may include a cover through which an outlet penetrates from the reactor in order to connect to a cannula. This has been done effectively. The cover may also vent steam generated by the heaters in the presence of the water typically used to activate such heaters.

The system may be configured for continual use by replenishing the reactants and replacing other components as needed. Alternatively, the system may be completely wrapped in a pre-packaged assembly. In one embodiment, a heat-shrinkable wrapping material may be used to seal the outer container of an apparatus in accordance with the invention. Thus, this system may be rendered tamper-proof, while also being maintained in integral condition throughout its distribution, storage, and use.

In accordance with the foregoing, certain embodiments of an apparatus and method in accordance with the invention provide a topical medium that produces nitric oxide and provides a therapeutic concentration of nitric oxide delivered to a surface. Nitric oxide may thus be introduced to the skin, or a wound, of a subject in a controlled manner. Nitric oxide amounts may be engineered to deliver a therapeutically effective amount on the order of from comparatively low hundreds (e.g., 100-500) of parts per million, up to thousands of parts per million. For example, sufficient nitric oxide may be presented through topical application to provide approximately five hundred parts per million to the surface of a subject's skin.

One embodiment of an apparatus and method in accordance with the present invention may rely on equal amounts of a nitrite medium and an acidified medium formulated to provide a burst of nitric oxide, as well as a continuous amount of nitric oxide over a period of time. One embodiment of an apparatus and method in accordance with the present invention may provide a therapeutically effective amount of nitric oxide from a gel medium, which provides a therapeutically effective dose of nitric oxide over a relatively shorter length of time, from approximately thirty minutes up to about 3 hours.

One embodiment of an apparatus and method in accordance with the present invention may provide a therapeutically effective amount of nitric oxide from a lotion medium, which provides a therapeutically effective dose of nitric oxide over a relatively longer length of time, from about one hour up to about 6 hours. Reactants may include potassium nitrite, sodium nitrite or the like. The reaction may begin upon combination of the nitrite medium and the acidified medium.

An apparatus and method in accordance with the invention may be used for a variety of purposes, including without limitation, disinfecting and cleaning surfaces, increasing localized circulation, facilitating healing and growth, dispersing biofilms, and providing analgesic benefits.

### BRIEF DESCRIPTION OF THE DRAWINGS

The foregoing features of the present invention will become more fully apparent from the following description, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a schematic view of one embodiment of an apparatus in accordance with the invention to generate nitric oxide and control the flow and concentration of nitric oxide delivered;

FIG. 2 is a perspective view of a containment vessel, or cannister;

FIG. 3 is a top perspective view of an open containment vessel, or cannister;

FIG. 4A is a cross-sectional view of a containment vessel, or cannister;

FIG. 4B is a close-up view of the center, bottom of the cross-sectional view of the containment vessel to more clearly show the heat cartridge sleeve of the containment vessel;

FIG. 5 is a schematic view of an automated feedback system that can monitor and adjust the flow or concentration of nitric oxide provided to a ventilator system;

FIG. 6 is a schematic of a possible combination a nitrite medium and an acidified medium for production of a topical medium for topical application of nitric oxide therapy;

FIG. 7 is a schematic block diagram of a computer system for implementing a programmed control process for a system in accordance with the invention;

FIG. 8 is a schematic block diagram of a hardware suite implementing one embodiment of an analysis and control system for administering nitric oxide gas therapy to a subject;

FIG. 9 is a schematic block diagram of one alternative embodiment thereof;

FIG. 10 is a side elevation, cross-sectional, schematic view of a fluid boundary layer near a sensor;

FIG. 11 is a side elevation, cross-sectional view thereof showing vectored flows to decrease thickness of the boundary layer;

FIG. 12 is a side elevation, cross-sectional view of one embodiment of a needle valve for use in an apparatus in accordance with the invention; and

FIG. 13 is a chart showing examples of monotonic approaches to adjustment of independent and dependent variables by a metering valve controller in accordance with the invention.

FIG. 14 is a side elevation view, in section, of a nitric oxide generator constructed in accordance with the teachings of the present invention.

FIG. 14A is an end elevation view of chemical mixture configuration.

FIG. 15 is a process flow diagram which demonstrates the utility of the nitric oxide generator illustrated in FIG. 14.

FIG. 16 is a high-level block diagram of one embodiment of a nitric oxide generator in accordance with the invention;

FIG. 17A is a front perspective view of one embodiment of an implementation of a nitric oxide generator in accordance with the invention;

FIG. 17B is a rear perspective view of the nitric oxide generator illustrated in FIG. 2A;

FIG. 18 is an exploded perspective view of the nitric oxide generator illustrated in FIGS. 2A and 2B;

FIG. 19 is a flow chart of one embodiment of a method for generating nitric oxide;

FIG. 20A is a high-level block diagram of one embodiment of a feedback system for use with a nitric oxide generator in accordance with the invention;

FIG. 20B is a high-level block diagram of another embodiment of a feedback system for use with a nitric oxide generator in accordance with the invention;

FIG. 21 is a flow chart of one embodiment of a method for creating a non-deliquescent nitric-oxide-producing tablet in accordance with the invention;

FIG. 22 is an enlarged perspective view of one embodiment of a tablet in accordance with the invention, showing the tablet's granular structure; and

FIG. 23 is a graph showing nitric oxide production of a tablet as a function of time and compression force.

FIG. 24 is a perspective view of one embodiment of an apparatus in accordance with the invention to generate nitric oxide from a chemically active source of nitric oxide, as a result of exposure to heat;

FIG. 25 is an exploded view of the apparatus of FIG. 24 for generating nitric oxide;

FIG. 26 is a top plan view of an insulating container for the apparatus of FIG. 24;

FIG. 27 is a side elevation view of the box-like container of FIG. 26;

FIG. 28 is an end, elevation, cross-sectional view of the container (box) of FIGS. 26-27;

FIG. 29 is a top plan view of a cover for the container of FIGS. 26-28;

FIG. 30 is an end elevation view of the cover of FIG. 29;

FIG. 31 is a side elevation view of the cover of FIG. 29;

FIG. 32 is a side elevation view of a vent for the portable nitric oxide device of FIG. 24;

FIG. 33 is a top plan view of the vent illustrated in FIG. 32;

FIG. 34 is a front elevation view of a triggering pin for the apparatus of FIG. 24;

FIG. 35 a is an end view of the pin of FIG. 34;

FIG. 35 b is a side elevation view of the pin of FIG. 34;

FIG. 36 is a bottom plan view of a guiding rod for holding a compression spring used in the trigger device of the apparatus of FIG. 25;

FIG. 37 is a side elevation view of the guide rod of FIG. 36;

FIG. 38 is a front elevation view of a spacer used in the piercing assembly of FIG. 25;

FIG. 39 is a top plan view of the spacer of FIG. 38;

FIG. 40 is a top plan view of the mounting assembly for a blade of the piercing assembly of the apparatus of FIG. 25;

FIG. 41 is an end elevation view of the mounting assembly or carrier for blades in the piercing assembly of FIG. 25, and corresponds to the apparatus of FIG. 40;

FIG. 42 is a side elevation view of the mounting assembly with blades in place, and corresponds to the apparatus illustrated in FIGS. 40-41;

FIG. 43 is a side elevation view of a base or base plate for supporting the blades in the piercing assembly of the apparatus of FIG. 25;

FIG. 44 is a top plan view of the base or base plate of the apparatus of FIG. 43;

FIG. 45 is a side elevation view of a cover plate for the blades in the piercing assembly of the apparatus of FIG. 25;

FIG. 46 is a top plan view of the cover plate of FIG. 45;

FIG. 47 is a side elevation view of a spring, used as a compression spring to drive the mounting assembly of FIG. 40, with the blades installed to operate the piercing assembly of FIG. 25;

FIG. 48 is a top plan view of one embodiment of a containment vessel operating as a reactor for the nitric oxide generation from the chemical species contained therein;

FIG. 49 is a side elevation view of the reactor's containment vessel of FIG. 48;

FIG. 50 is a side elevation view of one embodiment of a tube configured to operate as an outlet for the reactor vessel of FIG. 48;

FIG. 51 is a perspective view of one embodiment of a shrink-wrap sleeve that is applied to contain the overall enclosure of the apparatus of FIGS. 24-25;

FIG. 52 is a perspective view of the apparatus of FIGS. 24-25 with the open lid upside down;

FIG. 53 is a partially cutaway top perspective view of the apparatus of FIG. 52 open with the liquid bags removed for viewing of the internal piercing apparatus;

FIG. 54 is a graph showing data for the temperature rise in degrees Fahrenheit of the reactor of FIGS. 25, 52 and 53 using a variety of heaters including a single heater relying on water as the liquid, two standard heaters relying on water, and a single heater using salt water as the activating liquid;

FIG. 55 is a graph depicting the temperature response of the reactor of FIGS. 24-53 over time in both a single heater and double heater configuration;

FIG. 56 is a graph depicting the temperature response of the reactor of FIGS. 24-53 as a function of time when heated by a single heater and by double heaters; and

FIG. 57 is a chart depicting the released volume of nitric oxide from the reactor of FIGS. 24-53 superimposed over the temperature response thereof as a function of time.

FIG. 58 is a perspective view of one embodiment of a layup of layers in one apparatus in accordance with the invention;

FIG. 59 is an end, cross-sectional view of an apparatus in accordance with FIG. 58;

FIG. 60 is an end, cross-sectional view of an alternative embodiment of a two-part apparatus for introducing nitric oxide from reacting gels in accordance with the invention;

FIG. 61 is a top plan view of one embodiment of a mechanism for connecting the substrates under the gel strips in accordance with the invention;

FIG. 62 is an end, cross-sectional view of one embodiment of a packaging scheme for one embodiment of an apparatus in accordance with the invention;

FIG. 63 is a perspective view of one embodiment of an alternative, single-substrate apparatus formed in a method in accordance with the invention; and

FIG. 64 is a schematic block diagram of one embodiment of a method in accordance with the invention.

FIG. 65 is a perspective view of one embodiment of a system for generating and delivering nitric oxide in accordance with the invention;

FIG. 66 is an exploded view of alternative, cross-sectional, end views of the distributor of FIG. 65;

FIG. 67 is a perspective view of various alternative embodiments for a reaction chamber for the apparatus of FIG. 65;

FIG. 68 is a partially cut-away, perspective view of one embodiment of a reactor for use in the apparatus of FIGS. 65-67; and

FIG. 69 is a schematic block diagram of one embodiment of a method in accordance with the invention.

FIG. 70 is a schematic diagram illustrating one embodiment of an anti-microbial device in accordance with the present invention;

FIG. 71 is a schematic block diagram illustrating one embodiment of an anti-microbial method in accordance with the present invention;

FIG. 72 is a schematic block diagram of one embodiment of an experimental method in accordance with the present invention;

FIG. 73 is a table displaying data collected using the experimental method of FIG. 72;

FIG. 74 is a table displaying the reductions in bacterial growth achieved using the experimental method of FIG. 72; and

FIG. 75 is a table displaying the average reductions in bacterial growth achieved using the experimental method of FIG. 72.

### DETAILED DESCRIPTION OF THE PREFERRED EMBODIMENTS

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings.

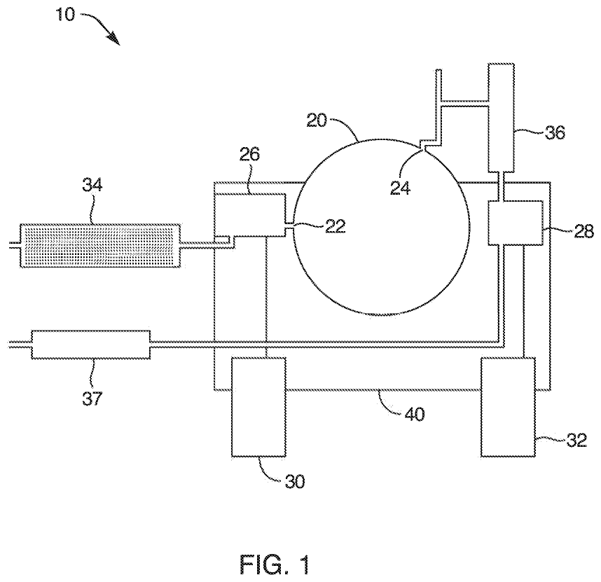

Referring to FIG. 1, a nitric oxide generator 10 may include a first pump 26 that draws air through an activated carbon filter 34 and pressurizes the reaction chamber 20, or reactor 20. The pump 26 provides filtered air for dilution with the nitric oxide to be generated. The pump 26 pumps air into the reactor 20 and pressurizes the reactor 20. Any device suitable for pumping air into and pressurizing the reactor 20 may be utilized.

The pump 26 may be controlled by a potentiometer 30, or the like. Using a potentiometer 30 allows the voltage to the pump 26 to be varied according to the desires of the user. The potentiometer 30 may include circuit boards that control the speed of the pump 26. Also, pump controls that control and measure the amperage to the pumps as opposed to the voltage may also be utilized when measuring the amperage is simpler, easier, or more useful for controlling the pump speed and power. Any device suitable for controlling the pump may be utilized.

The activated carbon filter 34 filters out oxygen and moisture from the inlet air. Again, any suitable device may be used to filter the inlet air appropriately. In another embodiment, the first pump 26 may pump air through the activated carbon filter 34 and then into the reaction chamber 20.

A reaction chamber 20 provides a suitable container for the reaction that produces the nitric oxide. The reaction chamber 20 can be of any suitable size or shape. The various configurations for a suitable reaction chamber 20, as well as the compounds and components used in the reaction, are described elsewhere hereinafter. However, compactness for portability and home use may be valuable.

A vent, or outlet 24, in the reaction chamber 20 allows air and nitric oxide to be drawn out of the reaction chamber 20. The outlet 24 may be configured to release excess pressure in the reaction chamber 20 by allowing air and nitric oxide to escape the system to the atmosphere. The outlet 24 may also be configured to direct the air and nitric oxide from the reactor to a first calcium hydroxide filter 36. The outlet 24 allows venting of the flow through the reactor and helps make sure the proper flow goes through the orifice. The system may provide means for applying a constant flow to the orifice and then venting overboard any remaining or excess flow of nitric oxide.

A second pump 28 draws air and nitric oxide through the first calcium hydroxide filter 36 away from the reaction chamber 20 for use in any type of nitric oxide therapy. The pump 28 further dilutes the nitric oxide with filtered air. The pump 28 may be controlled by a second potentiometer 32, or the like. Using a potentiometer allows the voltage to the pump 28 to be varied according to the desires of the user. The potentiometer may include circuit boards that control the speed of the pump. Also, pump controls that control and measure the amperage delivered to the pumps as opposed to controlling the voltage as described above. Any device suitable for controlling the pump may be utilized. The calcium hydroxide filter 36 absorbs or otherwise filters out moisture and scavenges nitrogen dioxide (NO₂) from the outlet air. Again, any other suitable device may be used to filter or otherwise clean the outlet air appropriately.

A line from the second pump 28 is used to conduct nitric oxide away from the reactor 20 and deliver the nitric oxide for use in various nitric oxide therapies. An orifice at one end of this line is used to restrict and control the flow of nitric oxide. The nitric oxide travels from the second pump 28 through this line, through the orifice, and through a second calcium hydroxide filter 37.

This line from the second pump to the orifice may be a ⅛ inch stainless steel line that carries gas and resists heat and corrosion. Any line used in this system may be a stainless steel line that carries gas and resists heat and corrosion, or any suitable device or material that can conduct the flow of gas in an acceptable manner. Also, any line in the system may be of silicone tubing that is resistant to heat, alcohol, and castor oil. Moreover, any line in the system may be composed of any material that is suitable for the intended purpose, including without limitation, stainless steel, medical grade silicone, plastic, or the like.

The orifice used to restrict and control the flow of nitric oxide may have an aperture from about 2 to about 10 mils, and typically about 0.004 inches in diameter. Any suitable aperture that will restrict and control (e.g., effectively meter) the flow of nitric oxide at a desired level. For example, orifice or aperture may typically be of any size from approximately 0.003 inches to 0.009 inches in diameter.

Finally, the second calcium hydroxide filter 37 removes any remaining moisture and nitrogen dioxide from the gas exiting the reactor 10. After passing through this second calcium hydroxide filter 37, the nitric oxide is ready for use with any variety of nitric oxide therapies. Also, the nitric oxide may be diluted with the air delivered to the patient.

The nitric oxide reactor 10 may include a cover 40 to contain the components of the reactor. The cover 40 may be any suitable shape and material and may be designed to allow access to the components of the reactor 10. The cover 40 may also be designed to enclose a reactor 10 intended for a single use by a patient. Such a single use reactor may be discarded or returned to an appropriate facility for recycling the reactor and its components.

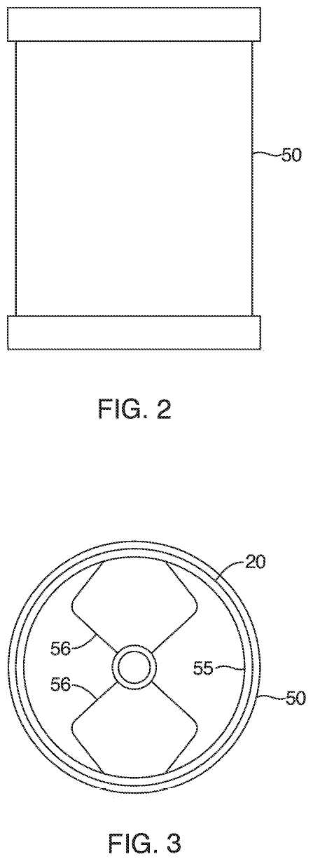

Referring to FIG. 2, in one embodiment the reaction chamber 20 may be contained within a containment vessel 50, or cannister 50. The top of the containment vessel 50 may be configured to be secured, such as by being screwed on to the containment vessel 50, to close or seal the reaction chamber, or unscrewed to allow access to the reaction chamber. The containment vessel may be heated by any suitable heating element. The containment vessel may be of any suitable configuration and may be made of any suitable material, such as stainless steel.

Referring to FIG. 3, in one embodiment the reaction chamber 20 may include fins 56, fin-like structures 56, in contact with the heating element 58 of the reaction chamber 20 and the outside wall 55 of the reaction chamber 20. These fins 56 dissipate the heat of the reaction and facilitate a complete nitric oxide reaction and use of all the reactants. These fins 56 may be composed of any serviceable heat transfer material that will not interfere with the reaction in the reactor and will stay in contact with the heating element 58 and the outside wall 55 of the reactor. Fins 56 may be designed to provide a constant contact force between the heater in the reactor and the wall of the reaction chamber 20. Fins 56 may be intimately bonded or may be described as “spring-loaded” fins in forced contact with the walls of the reaction chamber 20. The fins 56 are especially helpful when the reactants for the nitric oxide reaction include a powder, in which conductive heat transfer through the reactants is comparatively poor.

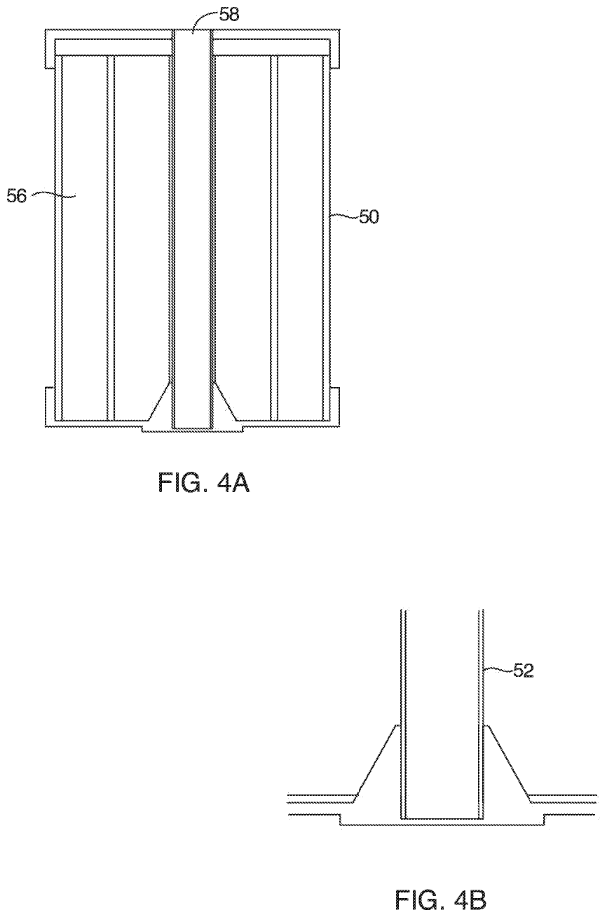

Referring to FIG. 4A, in one embodiment the reaction chamber 20 may be configured to allow for a heating element 58, or cartridge, extending axially along the containment vessel 50. Referring to FIG. 4B, the containment vessel 50 may also include a heat cartridge sleeve 52 to accommodate the heating element 58, or cartridge.

In one embodiment the formulation for the reactants may include the following: approximately 2.3 kg of calcined chromium oxide (Cr₂O₃) or approximately 51% of the granulation, approximately 1.6 kg of sodium nitrite (NaNO₂) or approximately 34.7% of the granulation, and approximately 0.65 kg of sodium nitrate (NaNO₃) or approximately 14.4% of the granulation. These amounts can be adjusted to provide an optimal production of nitric oxide. Generally, the amounts for the respective components may be adjusted plus or minus 10% of the granulation.

Calcined products are best stored under vacuum. The components are best ground to produce a loose granulation passing through a 5 micron screen. Each of the components should go through a double grind separately. All the components should be ground together a third time. The resulting granulation should be stored under nitrogen (N₂) or under vacuum at a comparatively cooler temperature than room temperature (lower is better) and in low light or no light conditions.

In one embodiment, the concentration of nitric oxide delivered can be varied anywhere from 0 ppm to one million ppm. Principally, the nitric oxide may be diluted with outside air. However, the system may be configured such that the nitric oxide can be diluted with any designated gas. Excess gas or nitric oxide can be vented to the atmosphere. The concentration can be adjusted rapidly in order to respond to the protocols and parameters of a variety of nitric oxide therapies.

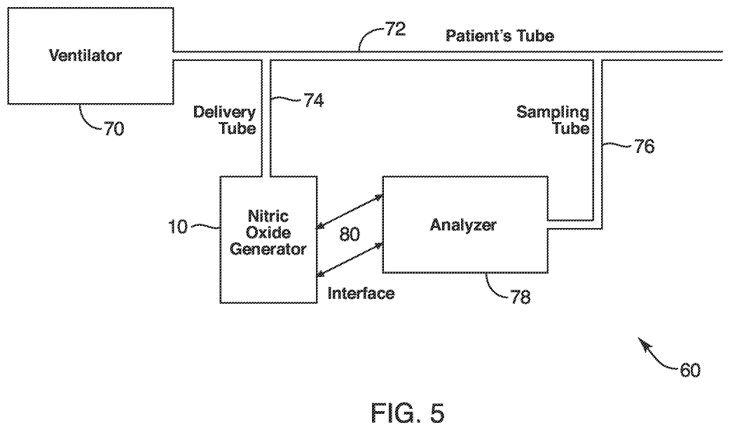

Referring to FIG. 5, in one embodiment, an integrated system 60 may be utilized to control and adjust the delivery of nitric oxide. Such a system may sample or measure the concentration of nitric oxide delivered to a user and then automatically adjust the amount of nitric oxide delivered to the air flow of the user. For example and not by way of limitation, a nitric oxide therapy may be delivered to a patient using a ventilator 70 with a breathing tube 72. After the nitric oxide is delivered to the air flow in the breathing tube through a delivery tube 74, a sample is taken through a sampling tube 76, or the air flow is measured, to determine the concentration of nitric oxide. Any device suitable for analyzing 78 or measuring the concentration of nitric oxide may be used. After a determination is made with regard to the concentration of nitric oxide, the amount of nitric oxide delivered to the air flow in the breathing tube can be adjusted by adjusting the controls of the nitric oxide dilution apparatus, such as adjusting the speed of the pumps or a bypass air inlet in the apparatus.

In one embodiment, an integrated system 60 includes a feedback loop. Measuring, adjusting, and controlling the concentration of nitric oxide may be monitored and controlled by an interface 80 device.

Again referring to FIG. 1, one embodiment of an apparatus and method in accordance with the invention may rely on a series of process steps constituting a method or process. For example, providing a pump may involve any one or more of the required tasks of identifying materials and determining the structural and mechanical characteristics for such a pump. Accordingly, providing a pump may involve design, engineering, manufacture and acquisition of such a device. Similarly, providing a potentiometer to control a pump by varying the voltage or current to the pump may involve identifying materials and determining the structural and mechanical characteristics for such a potentiometer. Accordingly, providing a potentiometer may involve design, engineering, manufacture, and acquisition of such a device.

Providing an activated carbon filter may involve identifying materials, selecting a shape, selecting a cross-sectional profile and active area, and determining the structural and mechanical characteristics for such a filter. Similarly, providing a calcium hydroxide filter may involve identifying materials, selecting a shape, selecting a cross-sectional profile, evaluating an active area, and determining the structural and mechanical characteristics for such a filter. Accordingly, providing any type of filter may involve design, engineering, manufacture and acquisition of such a device.

Providing a reactor may involve selection of materials, selection of a profile and of cross-sectional area, engineering, design, fabrication, acquisition, purchase, or the like of a reactor in accordance with the discussion hereinabove.

Providing reactants may include selection of reacting species, selecting a configuration, such as granules, powder, liquid, gel, a solution, multiple components to be mixed, or the like. Likewise, the particular configuration of a solidous configuration of reactants may involve selecting a sieve size for the particles. This size can affect surface area available to react, heat penetration distances, and times controlling overall chemical reaction rates. Thus, selecting or otherwise providing reactants for the reactor may involve consideration of any or all aspects of chemistry, reaction kinetics, engineering, design, fabrication, purchase or other acquisition, delivery, assembly, or the like.

Assembling the apparatus may also include the disposition of reactants within various locations within a reactor, system, or the like as discussed hereinabove.

Activating the reactants in the reactor may involve, either adding a liquid, mixing the reactant components together, dispersing individual reactants in respective solutes to provide solutions for mixing, adding a liquid transport carrier to dry ingredients in order to initiate exchange between reactants, heating the reactants, a combination thereof, or the like.

Likewise, activation of the reactants may also involve opening valves, opening seals, rupturing or otherwise compromising seals as described hereinabove, or otherwise moving or manipulating reactants with or without carriers in order to place them in chemical and transport contact with one another.

In certain embodiments, nitric oxide may be separated from the reactants themselves. For example, the concept of a molecular sieve as one mechanism to separate nitric oxide form other reactants and from other species of nitrogen compounds is possible. In other embodiments, pumps, vacuum devices, or the like may also tend to separate nitric oxide. Accordingly, in certain embodiments, a suitably sized pump may actually be connected to the reactor in order to draw nitric oxide away from other species of reactants or reacted outputs.

Conducting therapy using nitric oxide may involve a number of steps associated with delivery and monitoring of nitric oxide. For example, in certain embodiments, conducting therapy may involve activating a reactor or the contents thereof.

Monitoring may involve adding gauges or meters, taking samples, or the like in order to verify that the delivery of nitric oxide from the reactor to the user does meet the therapeutically designed maximum and minimum threshold requirements specified by a medical professional.

Ultimately, after the expiration of an appropriate time specified, or the exhaustion of a content of a reactor, a therapy session may be considered completed. Accordingly, the apparatus may be removed from use, discarded, or the like. Accordingly, the removal or discarding of the apparatus may be by parts, or by the entirety.

It is contemplated that the reactor may typically be a single dose reactor but need not be limited to such. Multiple-dose or reusable reactors may also be used. For example, the reactor may actually contain a cartridge placed within the wall. The internal structure of the cartridge may be ruptured in the appropriate seal locations, such as by a blade puncturing the seals by a mechanism on, in, or otherwise associated with the main containment vessel or wall, and thus activated. Accordingly, the reactor may be reused by simply replacing the cartridge of materials containing the reactant volumes.

A patient may also obtain the benefits of nitric oxide therapy by utilizing a topical application that generates nitric oxide. The nitric oxide may affect the surface to which the topical application is applied, and may be absorbed by a surface such as skin.

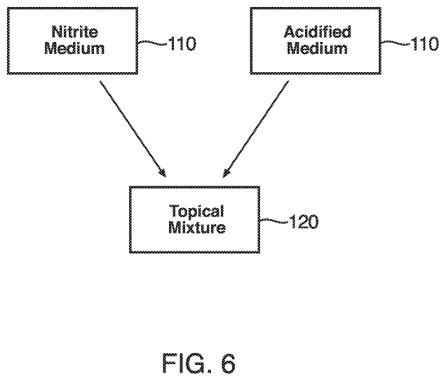

Referring to FIG. 6, two individual, separate, component media are provided. The first medium is a nitrite medium 100 and generally provides the nitrite reactants in some suitable form described herein above, such as sodium nitrite, potassium nitrite, or the like. The second medium is an acidified medium 110 and generally provides at least one acidic reactant in some suitable form, such as citric acid, lactic acid, ascorbic acid, or the like. Reaction rate and pH control are best achieved by using a mixture of multiple food-grade acids. When approximately equal amounts of the two individual components (media) are combined into a topical mixture 120, a reaction is initiated that produces nitric oxide.

Two containers may be provided, each container is capable of dispensing a suitable amount of a given medium (one of the two to be mixed). The containers may be identical in structure and composition, but need not necessarily be so. The containers may dispense the medium by a pump action, such as is common with lotions and soaps. The containers may dispense the medium by a squeezing or shaking action, such as is common with viscous or thixotropic shampoos, condiments, colloidal suspensions, gels, and other compositions.

The medium may be any suitable medium for containing and dispensing the reactants, for example, the medium may be a gel or a lotion. A gel may be obtained by including a water-soluble polymer, such as methyl cellulose available as Methocel™, in a suitable solution. A lotion used to suspend the reactants for a nitrite lotion medium and an acidified lotion medium may be selected such as the Jergens® brand hand and body lotion. For best results, the media holding a matched pair of reactants should be essentially the same. The chemical characteristics of the media may not be strictly identical, but the physical compositions should be essentially the same so as to mix readily and not inhibit the reaction.

For example, a nitrite gel medium may have a slightly acidic to neutral pH while an acidified gel medium may have a more acidic pH than the corresponding nitrite gel medium. Using a nitrite gel medium with an acidified lotion medium may not provide optimal results. Using different media may not provide the best rates for desired results, but would probably not be dangerous.

Generally, a topical application of nitric oxide may be provided by mixing equal amounts of a nitrite medium 100 and an acidified medium 110. The mixture 120 is then applied to the intended surface. The mixture 120 may be applied to a person's skin, or even an open wound.

The mixture 120 provides nitric oxide to the intended surface. As the nitrite medium 100 is mixed with the acidified medium 110, the reduction of nitrite by the acid(s) leads to the release of nitric oxide. The exposure to nitric oxide may serve a variety of purposes.

A topical mixture 120 that produces nitric oxide may be used for antimicrobial, antifungal, or similar cleaning purposes. Infectious diseases are caused by pathogens such as bacteria, viruses, and fungi. Antibacterial soaps can kill some bacteria, but not necessarily all bacteria. A topical mixture as described has been shown to kill as many as, and more, bacteria compared to commercially available antibacterial soaps or hospital-based instant hand antiseptics.

A topical mixture 120 that produces nitric oxide may be used for localized analgesic purposes. The analgesic effect nitric oxide may be provided via topical application.

A topical mixture 120 that produces nitric oxide may be used for anti-inflammatory purposes. A topical mixture that produces nitric oxide may also be used to disperse a biofilm. Biofilms are colonies of dissimilar organisms that seem to join symbiotically to resist attack from antibiotics. Nitric oxide signals a biofilm to disperse so antibiotics can penetrate the biofilm. It is also believed that nitric oxide interferes with the uptake of iron.

A topical mixture 120 that produces nitric oxide may be used to help heal various kinds of wounds. Tests have been performed wherein a topical mixture that produces nitric oxide as described herein is applied regularly to an open wound that is generally resistant to healing. The wound was seen to show significant healing within a few weeks.

For example, a person in Canada had poor circulation and unresponsive diabetic ulcers on the person's feet. The person was immobilized and in a wheel chair, and had been scheduled for amputation to remove the person's foot about a month after this experiment began. A topical mixture 120 that produces nitric oxide was applied to the diabetic ulcers once a day. The person soaked the effected foot in a footbath solution that produces nitric oxide for approximately twenty minutes once every four days. Within two weeks the person was able to walk and go out in public. Within 4-6 weeks, the person was mobile and had achieved a substantially complete recovery. Meanwhile, the scheduled amputation was cancelled.

It was shown that a topical mixture that produces nitric oxide will kill squamous cells, pre-cancerous cells, if the concentration of nitric oxide is high enough. Tests intending to show that a topical mixture that produces nitric oxide would grow hair based in part on the increase of blood flow that accompanies application of nitric oxide actually showed that nitric oxide in as high doses provided as described herein above did kill squamous cells.

The nitrite medium 100 may be formulated in any suitable medium and the concentration of reactants can be adjusted as desired as long as the intended reaction and sufficient concentrations of nitric oxide is obtained. For example, a suitable tank may be charged with distilled/deionized water (94.94% w/w) at room temperature (20°−25° C.). Sodium nitrite (3.00% w/w) and Kathon CG (0.05% w/w) may be dissolved in the water. Methocel™ (HPMC, cold dispersable; 1.75% w/w) may be stirred into the water until no lumps are present. Sodium hydroxide (10N to approximately pH 8; 0.09% w/w) may be rapidly stirred into the water to thicken, and care should be taken to avoid trapping air bubbles that can occur as a result of higher shear mixing.

EDTA, Na4 salt (0.10% w/w) may be stirred into the water until dissolved. Citric acid (crystalline; 0.08% w/w) may be added to adjust the mixture to a pH of 6.0. Small quantities of sodium hydroxide may be used to adjust the pH as needed. The individual percentages may be adjusted as desired for the best results.

The acidified medium 110 may be formulated in any suitable carrier and the concentration of the reactants can be adjusted as desired as long as the intended reaction and sufficient concentrations of nitric oxide are obtained. For example, a suitable tank may be charged with distilled/deionized water (89.02% w/w) at room temperature (20-25° C.). Kathon CG (0.05% w/w) may be dissolved in the water. Methocel™ (HPMC, cold dispersable; 1.75% w/w) may be stirred into the water until no lumps are present. Sodium hydroxide (10N to approximately pH 8; 0.09% w/w) may be rapidly stirred into the water to thicken, and care should be taken to avoid trapping air bubbles that can occur as a result of higher shear mixing.

EDTA, Na4 salt (0.10% w/w) may be stirred into the water until dissolved. Stirring may continue until the Methocel™ is completely hydrated. Lactic acid (85% liquid solution; 3.00% w/w) and ascorbic acid (USP, crystalline; 3.00% w/w) may be stirred in until completely dissolved. Citric acid (crystalline; 3.00% w/w) may be added to adjust the mixture to a pH of 6.0. Small quantities of sodium hydroxide may be used to adjust the pH as needed. The individual percentages may be adjusted as desired for the best results.

The use of at least two acids in producing the acidified medium 110 may improve the shelf life of the acidified medium 110. Generally maintaining a pH of from about 3 to about 5 or above (so long as not too caustic for skin) has been found very useful in maintaining the shelf life of the product.

A topical mixture 120 that produces nitric oxide has been shown to be effective in cleaning and disinfecting hands. For example, three sets of volunteers, with approximately 26 people in each set, participated in a test to determine the effectiveness of nitric oxide as a cleaning and disinfecting agent. The right and left hands of each person in each set of volunteers were swabbed with cotton-tipped applicators prior to any type of washing. The applicators were plated onto nutrient blood agar petri dishes using the three corner dilution method.

Each set of volunteers washed their hands using separate soaps for washing. The first set of volunteers washed their hands for thirty (30) seconds using a topical mixture 120 of equal parts of nitrite gel medium and acidified gel medium as described herein above. The second set of volunteers washed their hands for thirty (30) seconds using a commercial anti-bacterial agent Avagard™D. The third set of volunteers washed their hands for fifteen (15) seconds using Dial™ Complete Foaming Hand Wash, and then rinsed for fifteen (15) seconds and dried.

The right and left hands of each person in each set of volunteers were swabbed again with cotton-tipped applicators after washing. The applicators were plated onto nutrient blood agar petri dishes using the three corner dilution method. All the blood agar petri dishes were incubated for forty-eight (48) hours at 35° C. The results were tabulated based on a grading scale of bacteria colonization. The testing showed that a topical mixture that produces nitric oxide reduced the relative bacterial content by approximately 62%. Avagard™D reduced the relative bacterial content by approximately 75%. Dial™ Complete Foaming Hand Wash reduced the relative bacterial content by approximately 33%. Thus, a topical mixture that produces nitric oxide was found to be approximately twice as effective and cleaning and disinfecting hands than Dial™ Complete Foaming Hand Wash and almost as effective as Avagard™D.

It has been determined that the dose required to kill bacteria on a surface, such as a person's skin, is at least approximately 320 ppm of nitric oxide. A topical gel mixture of approximately three (3) grams of nitrite gel medium and approximately three (3) grams of acidified gel medium that produces nitric oxide has been shown to deliver approximately 840 ppm of nitric oxide. Similarly, a topical gel mixture of approximately three (3) grams of nitrite lotion medium and approximately three (3) grams of acidified lotion medium that produces nitric oxide has been shown to deliver approximately 450 ppm of nitric oxide.

Measurement, control, and stability of flows of nitric oxide are another matter. Timely and precise control is not available. Closed loop control is not used in therapy. Coarse (imprecise) control and no automatic feed back are the norm. Speed and precision over a wide range of flow rates is now available.

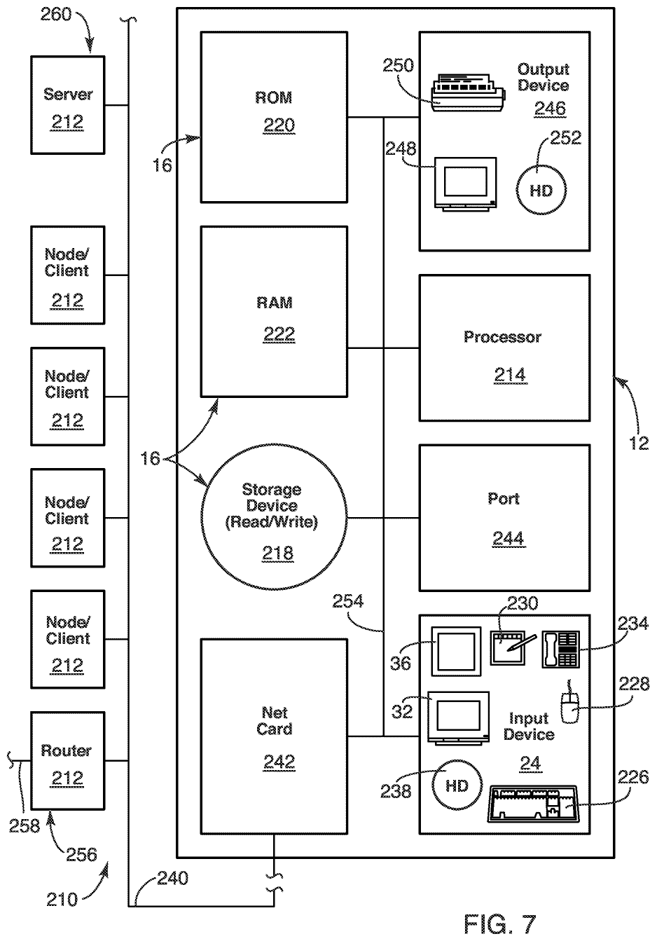

Referring to FIG. 7, a computer apparatus 210 or system 210 for implementing various aspects of the present invention may include one or more nodes 212 (e.g., client 212, computer 212). Such nodes 212 may contain a processor 214 or CPU 214. The CPU 214 may be operably connected to a memory device 216. A memory device 216 may include one or more devices such as a hard drive 218 or other non-volatile storage device 218, a read-only memory 220 (ROM 220), and a random access (and usually volatile) memory 222 (RAM 222 or operational memory 222). Such components 214, 216, 218, 220, 222 may exist in a single node 212 or may exist in multiple nodes 212 remote from one another.

In selected embodiments, the computer apparatus 210 may include an input device 224 for receiving inputs from a user or from another device. Input devices 224 may include one or more physical embodiments. For example, a keyboard 226 may be used for interaction with the user, as may a mouse 228 or stylus pad 230. A touch screen 232, a telephone 234, or simply a telecommunications line 234, may be used for communication with other devices, with a user, or the like. Similarly, a scanner 236 may be used to receive graphical inputs, which may or may not be translated to other formats. A hard drive 238 or other memory device 238 may be used as an input device whether resident within the particular node 212 or some other node 212 connected by a network 240. In selected embodiments, a network card 242 (interface card) or port 244 may be provided within a node 212 to facilitate communication through such a network 240.

In certain embodiments, an output device 246 may be provided within a node 212, or accessible within the apparatus 210. Output devices 246 may include one or more physical hardware units. For example, in general, a port 244 may be used to accept inputs into and send outputs from the node 212. Nevertheless, a monitor 248 may provide outputs to a user for feedback during a process, or for assisting two-way communication between the processor 214 and a user. A printer 250, a hard drive 252, or other device may be used for outputting information as output devices 246.

Internally, a bus 254, or plurality of buses 254, may operably interconnect the processor 214, memory devices 216, input devices 224, output devices 246, network card 242, and port 244. The bus 254 may be thought of as a data carrier. As such, the bus 254 may be embodied in numerous configurations. Wire, fiber optic line, wireless electromagnetic communications by visible light, infrared, and radio frequencies may likewise be implemented as appropriate for the bus 254 and the network 240.

In general, a network 240 to which a node 212 connects may, in turn, be connected through a router 256 to another network 258. In general, nodes 212 may be on the same network 240, adjoining networks (i.e., network 240 and neighboring network 258), or may be separated by multiple routers 256 and multiple networks as individual nodes 212 on an internetwork. The individual nodes 212 may have various communication capabilities. In certain embodiments, a minimum of logical capability may be available in any node 212. For example, each node 212 may contain a processor 214 with more or less of the other components described hereinabove.

A network 240 may include one or more servers 260. Servers 260 may be used to manage, store, communicate, transfer, access, update, and the like, any practical number of files, databases, or the like for other nodes 212 on a network 240. Typically, a server 260 may be accessed by all nodes 212 on a network 240. Nevertheless, other special functions, including communications, applications, directory services, and the like, may be implemented by an individual server 260 or multiple servers 260.

In general, a node 212 may need to communicate over a network 240 with a server 260, a router 256, or other nodes 212. Similarly, a node 212 may need to communicate over another neighboring network 258 in an internetwork connection with some remote node 212. Likewise, individual components may need to communicate data with one another. A communication link may exist, in general, between any pair of devices.

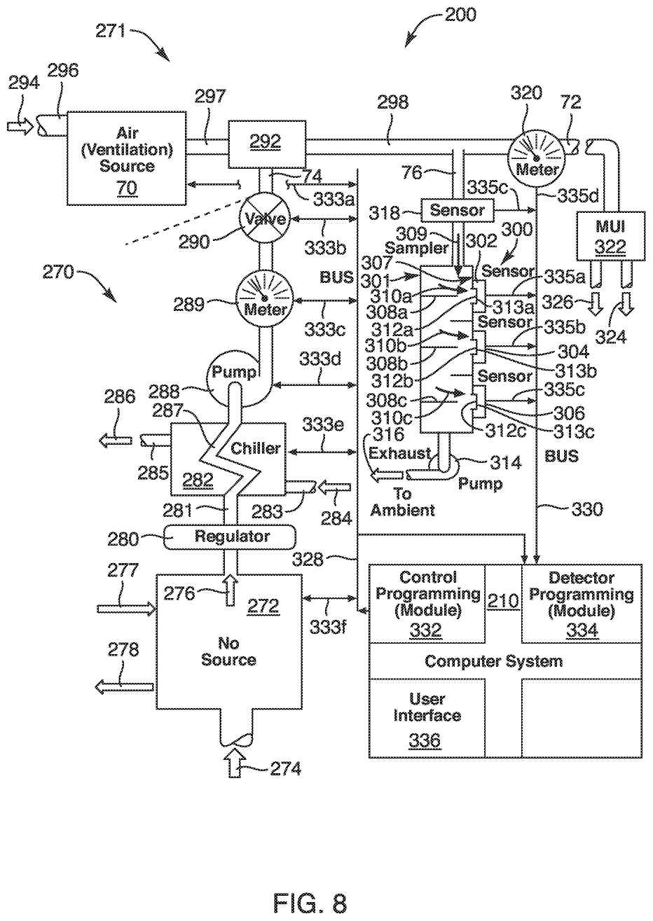

Referring to FIG. 8, a nitric oxide delivery system 200 or system 200 may rely on a computer system 210, embedded therein or otherwise operably connected thereto, in order to deliver a therapeutic gas through a gas titration system 270. Gas flows 276 including nitric oxide, other gases, or both, are measured and delivered into the flow 297 and outputs 324 of a breathable gas system 271 or air source 70 for ventilation of a subject. Therefore, a therapeutic gas titration system 270 (or simply a therapeutic gas system 270 or system 270) may interface with a breathable gas system 271. Typically, the therapeutic gas system 270 begins with a source 272 for the therapeutic gas, typically nitric oxide. The source 272 may actually provide for materials 274 input into the source 272, such as the generator 10 discussed hereinabove. In other embodiments, the source 272 may be bottled nitric oxide gas, or a pressurized tank of nitric oxide gas.

In certain embodiments, such as the generator 10 hereinabove, input materials 274 may be provided as well as other inputs 277, such as electrical power, thermal energy, other chemical constituents, other supporting materials, or the like. The result from the source 272 is an output 276 of substantially pure nitric oxide 276. Meanwhile, to the extent that materials 274 or other inputs 277 may require a discharge 278 of waste products, thermal energy rejection from thermodynamic processes, chemical processes, or the like, they may result in discharges 278.

In many embodiments, a source 272 will interface with the remainder of the therapeutic gas system 270 by a regulator 280 controlling pressure to a predetermined value for introduction into the remainder of the system 270.

In the illustrated embodiment, a line 281 may pass the therapeutic gas into a chiller 282. A chiller 282 is significant in that it has been found effective to reduce the temperature and pressure at which nitric oxide is handled. Decompression and cooling have been shown effective to reduce secondary reactions of nitric oxide into nitrogen dioxide or NO₂. Again, in the illustrated embodiment, the chiller 282 may therefore be used, particularly if the source 272 is a thermally driven generator 10.

The chiller 282 may provide an inlet 283 whereby coolant 284 is introduced in a cross-flow, counter-flow, concurrent-flow, or other arrangement in order to cool the therapeutic gas 276. The coolant 284 passing through the inlet 283 will be used to chill the therapeutic gas 276 received from the source 272. The warmed flow 286 of coolant 284 will exit through the port 285 or outlet 285 after passing over the coils 287 or passes 287. For example, good heat exchanger design may dictate more than one passage of the coils 287 through the interior of the chiller 282 for extended exposure to the coolant flow 284.

Typically, the pump 288 may be positioned downstream of the source 272, and often downstream of the chiller 282. One purpose for the pump 288 drawing on the source 272 is to maintain minimum pressures in the lines 281, 74 in order to minimize reaction of nitric oxide into nitrogen dioxide, which is considered an undesirable oxide of nitrogen.

Typically, a meter 289 or flow meter 289, illustrated schematically only, will need positioned somewhere in the line 74 feeding the therapeutic gas 276 to the breathing line 297 or flow 297. However, the position of the meter 289 and valve 290 are not necessarily critical. For example, the positions of the meter 289 and valve 290 may be switched. Likewise, the pump 288 may be positioned downstream of one or both of the meter 289 and valve 290. The pump 288 is responsible to deliver therapeutic gas 276 through the line 74 into a mixer 292 or chamber 292 that receives both the therapeutic gas and breathing air 294. The breathing air 294 may be considered an intake material through a port 296 or inlet 296 drawn into a source 70 or ventilator source 70, also simply referred to as an air source 70. This ventilator 70 is responsible to provide clean, breathable air, typically ambient air 294, and not typically pure oxygen. However, various processes may be employed to provide a flow 297 or feed 297 that will be directed to a subject (e.g., patient).

The meter 289 is best served by a float valve 289 sometimes referred to as a “pea valve” 289 that relies on a variable flow passage based on the elevation of an aerodynamically lifted indicator. This light weight indicator rests in a flow passage having a variable cross-sectional area depending on the altitude at which the float rides. The readout of the system 289 may be manual, electronic, or rely on other mechanism. The float height is a function of “pressure head” and flow rate. However, the pea valve system 289 has been found to produce precision with a minimum of obstruction, as compared with other types of metering valves 289. Thus, the flow meter 289 provides a measurement for the actual volumetric flow rate of the therapeutic gas 276 through the line 74.

The valve 290 is a metering valve. The presence of the meter 289 with the metering valve 290 is not redundant. The purpose of the meter 289 is to determine the actual volumetric flow rate of the therapeutic gas. Meanwhile, the metering valve 290 is a control element 290 that precisely controls exactly the amount of therapeutic gas flow 276 that will be permitted. More will be discussed hereinbelow regarding the metering valve 290 or control valve 290.

Ultimately, the line 74 delivers the therapeutic gas into a chamber 292 that operates as a mixer 292 with the flow 297 or line feed 297 from the ventilator 70 directed toward the subject. Thus, the flow 298 or line 298 is a mixture of the air flow 297 from the ventilator 70 and the therapeutic gas flow from the line 74 delivered from the therapeutic gas system 270.

A detector system 300 involves a series of sensors 302, 304, 306. In the illustrated embodiment, each of the sensors 302, 304, 306 operates to detect a different gas, here, nitric oxide, nitrogen dioxide, and oxygen, respectively. The sensors operate within a manifold 301 wherein each of the sensors 302, 304, 306 is mounted in or at a wall 307 of the manifold 301. Meanwhile, the operation of the sensors 302, 304, 306 and the metering by the metering valve 290 are operated in a new manner in order to obtain the precision and responsiveness required for a system 200 in accordance with the invention. For example, the metering valve 290, even when selected to be the most precise available, operating at the pressures important to the therapeutic gas delivery system 270, is wholly inadequate. That is, the precision of the best metering valves 290 available provides inadequate metering when operating in the realm of pressures (e.g., less than an atmosphere, sometimes less than a third or a fourth of that) desired for minimizing consequent reactions of the nitric oxide.

Of particular problematic nature is the backlash or tolerance that exists because the valve 290 is a threaded needle valve 290 in one currently contemplated embodiment. Necessarily, threads must have tolerances. Tolerances create slack, slop, hysteresis, or backlash. Hysteresis is the phenomenon that a movement or a change between a first state and a second state does not travel the identical path in both directions between those two states. Hysteresis is a principle understood and documented in electrical engineering and mechanical engineering literature. In the metering valve 290, hysteresis refers to the fact that movement of a needle valve in one direction is driven by engagement of respective threads on the shaft of the needle and matching, mutually engaging threads on a surrounding housing. Movement in an opposite direction requires engagement of different faces on opposite sides of the threads of the shaft and the threads of the housing. Thus, that slack or tolerance generates a mechanical hysteresis, which is excessive, in view of the precision required for a system 200 in accordance with the invention.

Likewise, the sensors 302, 304, 306 are insufficiently responsive to make measurements quickly and precisely when used in their typical manner. Each of the sensors 302, 304, 306, may operate sufficiently precisely when detecting gases in a contained vessel, tank, line, or the like operating in a steady state. For example, systems may be calibrated to account for the fact that diffusion of chemical species toward a sensor 302, 304, 306 may be accommodated as a matter of course.

Here, the sensors 302, 304, 306 are used as a feedback mechanism to control the valve 290. A rapid, transient response is needed. A combination of the diffusion gradient in a boundary layer near a face 312a, 312b, 312c of a sensor 302, 304, 306, respectively, is completely insufficient a process for sufficiently timely, accurate control. For example, typical meters 289 expect to flow an amount of nitric oxide gas on the order of about 100 parts per million in order to apply therapeutically appropriate concentrations of nitric oxide in a flow 294 or feed line 297 of breathing air 294 treated with a therapeutic gas.

It is desired, in contrast, to provide metering down to single digits of parts per million precision in the feed 298 or line 298 running to the subject. Also, it is desired to increase the concentrations up to hundreds, even thousands of parts per million in some configurations. Typically, adult concentrations may be on the order of five or six hundred parts per million and topical applications (e.g., disinfection immersion, wound immersion in nitric oxide gas flow, etc.) may involve thousands of parts per million.

Thus, in an apparatus and method in accordance with the invention, the hysteresis of the best valves 290 available coupled with the concentration gradients near the sensing faces 312 of the sensors 302, 304, 306 combine to put the needed precision completely out of reach. One should remember a reference numeral followed by trailing a letter refers to the item corresponding to the number, but the particular instance thereof corresponding to the trailing letter. Thus, we may speak of faces 312, applying to all versions or instances of the face 312, whereas the faces 312a, 312b, 312c may refer to specific instances corresponding to each of the respective sensors 302, 304, 306.

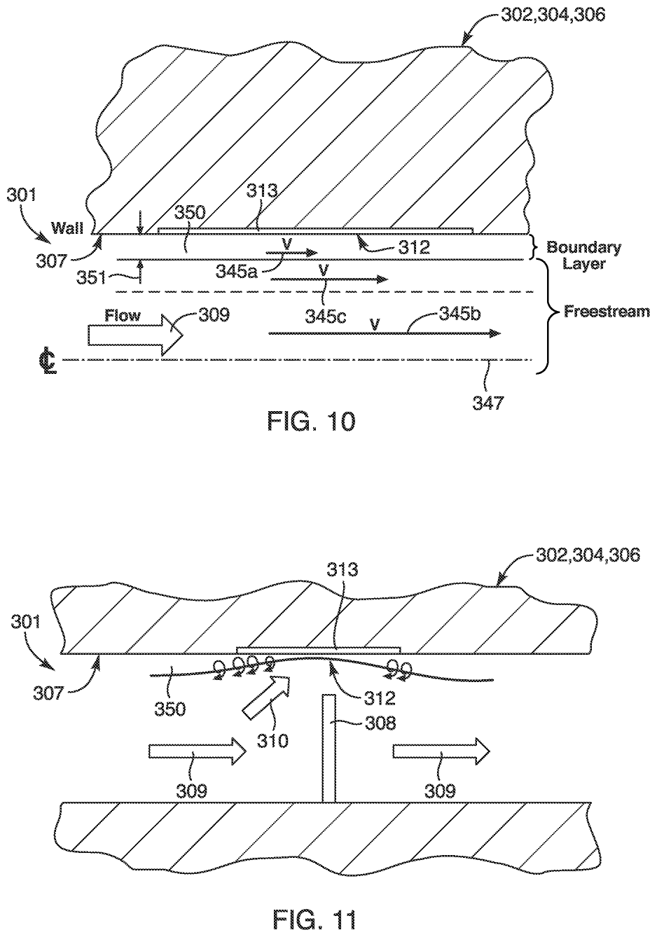

In the illustrated embodiment, the manifold 301 or chamber 301 may be constructed in a variety of configurations. However, it has been found that a mechanism is required to effectively thin or virtually destroy (reduce to some minimum value) the aerodynamic or hydrodynamic boundary layer (350, see FIGS. 10-11) against the faces 312. To that end, a series of diverters 308 divert the incoming flow 309 to a vectored flow 310 for each respective sensor 302, 304, 306. Each of the diverters 308a, 308b, 308c, corresponding to each of the vectored flows 310a, 310b, 310c, effectively strips the boundary layer 350 away from the face 312a, 312b, 312c of each of the respective sensors 302, 304, 306. More will be discussed hereinbelow regarding the operation of diverters.

However, a barrier, vane, ramp, nozzle, baffle, or other device to redirect flows 309 into vectored flows 310 provides two improvements to the performance of the sensors 302, 304, 306. First, because the boundary layer 350 is thinned, the time response for diffusion of the sensed gases approaching each of the faces 312 is dramatically reduced. The distance is reduced and the time for transport across the boundary layer 350, to the extent that any boundary layer 350 exists, is greatly reduced. Shear, mixing, and thinning all result. This improves both the accuracy, and the time response to a much better performance than would normally be expected or possible in the sensors 302, 304, 306.

Typically, the sensors 302, 304, 306 each have a sensing material 313 electronically coupled to a signal (e.g., voltage) that will be read out to a computer system 10 by the sensor 302, 304, 306. That output is a response to the presence and concentration of the specified chemical constituent being sensed. Thus, diffusion through a boundary 312 or face 312 of the sensing material 313 from the flow 310 past the face 312 is effective. With regard to the chemical process or electrochemical performance of the face 312 and material 313 for any sensor 302, 304, 306, a large barrier to diffusion is the diffusion through the boundary layer 350 within the fluid flow 309 within the manifold 301.

A pump 314 may operate upstream or downstream of the manifold 301. Regardless, the significance of the pump 314 is to draw through the line 76, a small flow 309 (comparatively speaking, with respect to flows 276, 294) from the flow 298 or line 298 that will be delivered to a subject. To that end, the pump 314 discharges an exhaust 316 overboard to the ambient. The quantity of the flow 309 is small and environmentally insignificant.

Meanwhile, one or more sensors 318 may be placed in the line 76 to detect any obstruction that may interfere with proper flow through the manifold 301. In the illustrated embodiment, the sensors 318 may include a pressure sensor, a flow meter, or the like. Thus, if the line 76 becomes occluded at any point between the feed line 298 and the pump 314, that obstruction may be timely detected and cured. Thus, the sensors 318 may include one or more sensors as deemed appropriate. A single detector of pressure has been found effective. Meanwhile, a single detector indicating flow may also serve equally well.

A meter 320 may typically be a float valve type of meter that effectively floats a comparatively light weight solid object within a vertical passage of variable cross-sectional area. Thus, with larger aerodynamic or hydrodynamic head, the float (indicator) is driven further upward against gravity. The flow, meanwhile, with increasing elevation encounters a larger cross-sectional area providing additional bypass around the indicator. This provides a non-linear response varying from a comparatively smaller flow when the float is at a lower position to a comparatively much larger flow at higher elevations of the float where the cross-sectional area is substantially increased.

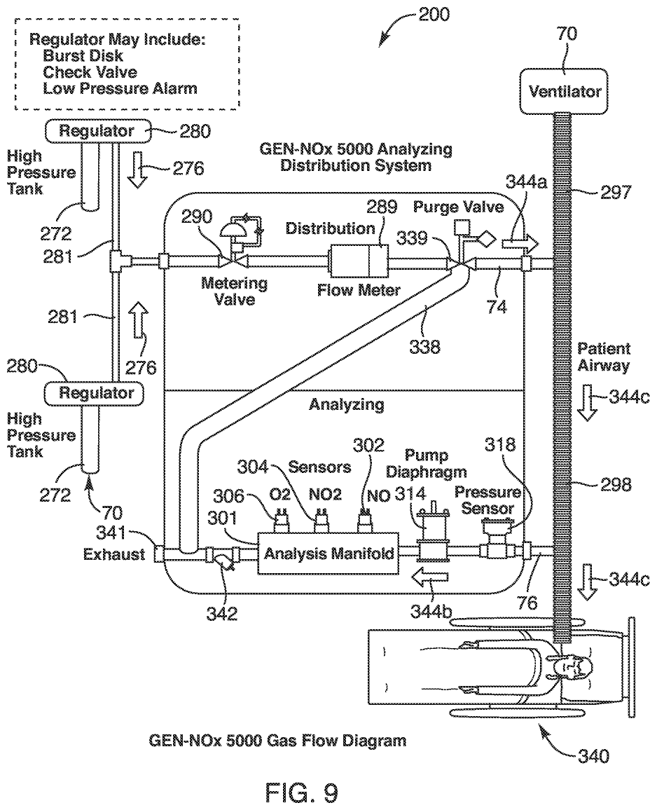

In a system 200 in accordance with the invention, a user interface 322 or mechanical user interface 322 may provide both a treatment flow 324 to a subject 340 (see FIG. 9) and an overboard discharge 326 or bypass flow 326. For example, a mechanical user interface 322 may be embodied as a breathing tent covering the upper portion of a body of an adult, an incubator tent covering a newborn infant, or the like. In other embodiments, the mechanical user interface 322 may be a mask 322, tube 322, or cannula 322 that provides a breathable gas flow 324 treated with the therapeutic gas 276 for breathing by a subject 340.

Mechanical devices such as the ventilator 70 and any driving mechanisms, such as pumps 288, fans 288, or blowers 288 associated with the ventilator 70 typically are not and cannot effectively or cost effectively be associated with the breathing process of an individual. Rather, a particular flow 72 will be delivered through the line 72 to a user interface 322 at the controlled rate. Note: any flow 297, 298, 72, may be designated by its unique line 297, 298, 72, respectively. Thus, any amount of the flow 324 used by a user will be intermittent according to a rate of breathing in and breathing out. The discharge 326 or overboard dumping 326 will accommodate the remainder of the flow 298 delivered to a subject. A tent must be vented, a cannula into the nostrils, but may be bypassed, and is, in fact, thrown overboard with each exhale by user.

In other embodiments, a mask, such as the CPAP mask or other masks delivering to mouth, nose, or both, may act as the mechanical user interface 322. The expression “mechanical” refers to the fact that this is not a data input, or even the chemical interface. Rather, the user interface 322 refers to the fact that mechanical devices move air, and direct its flow. Accordingly, a mechanical user interface 322 (e.g., mask 322, cannula 322, tube 322, CPAP 322, mouth piece 322, etc.) directs the flow 324 to a user, and accommodates the discharge overboard 326.

The therapeutic gas system 270 and the detector suite system 300 may both be operably connected to be controlled by computer system 210. In the illustrated embodiment, the ventilation source 70 may also be controlled by the computer system 210. However, this is not essential. However, it is much more valuable and much more important to control proper dosing of therapeutic gas into the flow 298. This will assure the provision of nitric oxide through the line 74 and the control of the components in the system 270 or subsystem 270. It also assures timely and accurate logging and detection from the detector suite 300.

Sensors 302, 304, 306, 318 should be precisely and timely controlled, read, and otherwise communicated with by processes executed by one or more computer 312 or processors 214 in a computer system 210 in accordance with the invention. In the illustrated embodiment, control programming 332 may be embodied as a control module 332 asserting control over the components in the therapeutic gas subsystem 270 or system 270. The inputs received from the various meters 289, 320, 333, valves 290, and sensors 302, 304, 306, 318 need to be received and processed by the detector programming 334 or detector module 334 in the computer system 210, which may be a single computer 312 or multiple, networked computer 312.

An individual operating the system 200 may set up its operation, monitor operation, and so forth including setting dosing, recording history, and the like. One may access the computer system through a user interface 336 including input systems such as a keyboard, touchscreen, number pad, mouse, and the like discussed hereinabove, as well as reading displays, monitors, alarms, and the like.

In general, a bus 328 or delivery bus 328 may include hard wiring 328, a conventional computer bus 328, or other communication link 328 permitting transmission of information to and from each of the components in the subsystem 270. Likewise, the detector suite 300 may communicate with a detector bus 330 or bus 330 providing information to the detector module responsible for processing those inputs. The delivery bus 328 may provide inputs from sensors involved with any component of the subsystem 270 to the detector module in order to process those inputs.

Command signals from the control module 332 directed to the components of the subsystem 270 may be passed along the bus 328. Any bus 328, 330 may be implemented as multiple buses 328, 330, or a single bus 328, 330, multiple wires, directly to devices, or the like. Thus, a mechanically fixed bus mechanism 328, 330 may be used, but in many environments, a more conventional computer data bus 328, 330 may serve to communicate between network aware devices or computer peripheral devices operating as components of the subsystem 270.

The connections 333 provide inputs for controlling various components as well as responses reading any detectors (e.g., 302, 304, 306, 318, etc.) provided in those components. For example, the connection 333a may communicate between the bus 328 and the ventilator 70. The communication connection 333b may pass control signals to the metering valve 290, and may report back data to the bus 328 ultimately directed to the detector module 334.

The communication link 333c or connection 333c provides information to, information from, or both, with respect to the flow meter 289. Similarly, the pump 288 may be controlled and monitored by a communication link 333d between the pump 288 and the bus 328. The chiller 282 may communicate to and from the computer system 210 over a communication link 333e. Similarly, a nitric oxide source 272 may receive control signals, report data, and the like to the computer system 210 over the bus 328 by means of a communication link 333f.

Again, each component need not have direct control or feedback control. Some systems such as a ventilator 70, may be set at a specific operating point or control position. Likewise, if a gas cylinder is used for the source 272, setting a regulator and metering valve may simply provide all the control that will be needed. However, in the illustrated embodiment, a regulator 280 as well as a metering valve 290 are both present, the latter being precisely controlled by the computer system 210.

Similarly, connections 335a, 335b, 335c communicate between the sensors 302, 304, 306, respectively, and the bus 330 serving the detector module 334. Typically, the detector module 334 may be thought of as the data acquisition module 334 responsible to pull in desired data logged from any point in the system 200, not simply the sensors 302, 304, 306.

Referring to FIG. 9, in one alternative embodiment of a system 200 in accordance with the invention, a source 272 may simply be a pressurized tank 272 equipped with a regulator 280 delivering a flow 276 or an output 276 in a set of lines 281. In the illustrated embodiment, multiple tanks 272 are illustrated, each of which may be provided with a check valve, a protection for over pressure, such as a burst disc, and likewise some indicator for approaching an empty condition, such as a low-pressure alarm. The regulators 280 regulate pressure from a tank 272 in order to provide it to the system 200 without damaging components and in order to distribute at a specific and constant rate.

In the illustrated embodiment, flows 276 through the line 281 deliver to a metering valve 290 and through a flow meter 289 a flow of the nitric oxide or other therapeutic gas. A purge valve 33 may initially divert to a purge line 338 any residual gas that is not the therapeutic gas and should be extracted from lines before operation. As a practical matter, for set up, calibration, initiation, and the like, a purge line 338 serviced by a purge valve 339 may serve to purge the line 74 of ambient air or whatever may exist within it. For example, other oxides of nitrogen may have formed from the remaining residual of nitric oxide when a system 200 was shut down.

As illustrated, the ventilator 70 provides breathing air along a line 297 representing a flow 344c or flow along a direction 344c. Meanwhile, the metering valve 290 discharges a flow 344a or a flow in a direction 344a to the line 297 for mixing. A sampling line 76 takes a small amount, not shown to scale, from the line 298, passing it by sensors 318 as described hereinabove, and driven by a pump 314, also described hereinabove. Ultimately, whether the pump 314 is upstream or downstream from the manifold 301, each of the sensors 302, 304, 306 as described hereinabove with its diverters 308 to each of the sensors 302, 304, 306 provide detection of species for analysis and feedback.

In the illustrated embodiment, the check valve 342 may assure that over pressure in the purge line 338 does not result in passing any undesired flow back into the analysis manifold 301 or from affecting the pressure and thereby changing the analysis. Ultimately, the exhaust port 341 discharges any waste from the purge line 338, as well as the flow 309 through the line 76 indicated by the direction 344b passing through the analysis manifold 301.

Ultimately, the subject 340 will receive from the line 298, through a mechanical user interface 322 the therapeutic gas 324 while any overage or overboard discharge 326 is passed to the environment.

Referring to FIG. 10, the operation of the manifold 301 in general will typically be illustrated by any particular sensor 302, 304, 306, having its boundary wall 307 containing a flow 309 therethrough. In the illustrated embodiment, the sensor face 312 on the sensor material 313 receives the species of chemical to be tested as diffused through the boundary layer 350, characterized by a thickness 351. Thus, the laminar boundary layer 350 or other boundary layer 350 will typically set up according to aerodynamic flow theory to cause a thickness 351 through which the species to be detected, measured, or “tested for” must diffuse.

Otherwise, the flow 309 has a velocity distribution illustrated by various velocities 345. The velocity 345a in the boundary layer is the slowest, and is, in fact, at a zero value at the wall 307. The velocity 345a in the boundary layer 350 varies from stationary at the wall 307, to some positive value greater than zero at the transition of the boundary layer 350 into the free stream 346. Meanwhile, the velocity 345c near but outside the edge of the boundary layer 350 is greater than the average or even maximum value of the velocity 345a in the boundary layer 350 itself. The bulk velocity 345b or maximum velocity 345b in the velocity profile within the free stream will typically be at a maximum along the center line 347 of the flow 309. The profile of all velocities 345 will be determined by the equations of fluid flow from engineering.

Referring to FIG. 11, in one aspect of an apparatus in accordance with the invention, the flow 309 is diverted by a diverter 308. The diverter 308 may be a ramp, a baffle, an obstruction, a nozzle, a channel, or any other director 308 that will redirect the flow 309 from passing through the manifold 301. The vectored flow 310 will impinge directly on the boundary layer 350 and the sensor surface 312 of the sensor material 313 in the sensors 302, 304, 306. Here, any one of the sensors 302, 304, 306 may be represented in the illustrated embodiment.

Each sensor 302, 304, 306 has a sensor material 313, and a sensor face 312 impinged upon by the vectored flow 310. The vector 310 effectively reduces to a value as close to zero as practical the thickness 351 of the boundary layer 350. Thus, the thickness 351 through which a tested species must pass, and the delay therefor has been minimized. This provides suitable speed of response, and less dependence on a steady state, calibration, and so forth. Thus, the dynamic response of the overall system 270 or subsystem 270 is greatly improved by access to more accurate, more timely, and more closely tracked concentration data for each of the tested species.

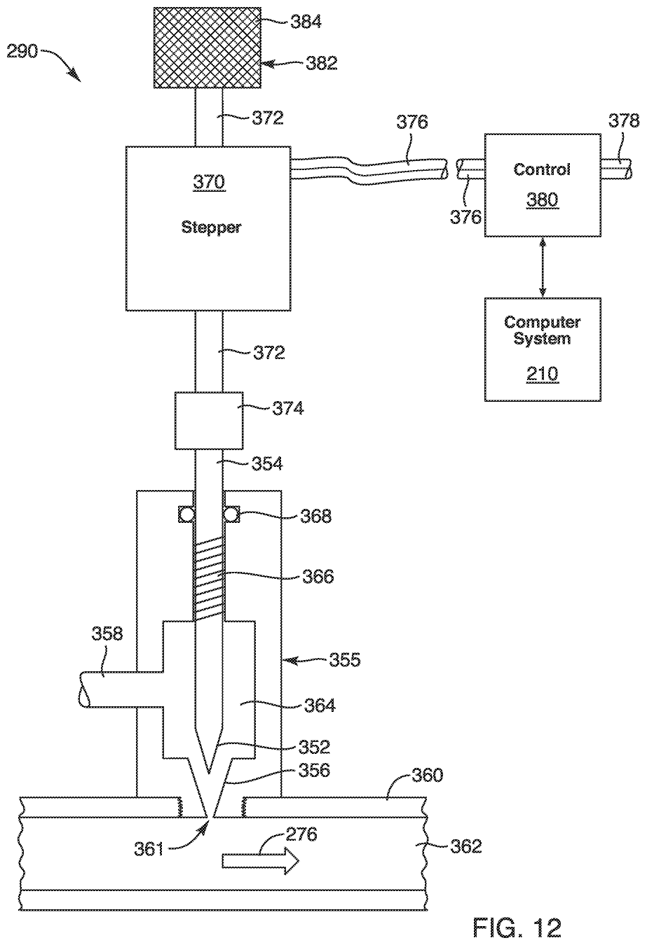

Referring to FIG. 12, in one embodiment of an apparatus and method in accordance with the invention, a metering valve 290 may be embodied as including a series of components operating to precisely meter, and to precisely control, the flow 276 of therapeutic gas through the metering therapeutic gas subsystem 270.

In the illustrated embodiment, a point 352 (needle 352) of a shaft 354 fits within a housing 355. The point 352 is machined, formed, or otherwise made to fit a seat 356 precisely. The seat 356 may be a separate component, or may be fabricated as part of the material of the housing 355. Typically, the seat 356 may be an insert 356 precisely formed and fitted into the housing 355 to receive the point 352 of the shaft 354. Ultimately, the port 358 may receive a source of material that will be metered into the line 362 or the flow 362 within the line 360 or conduit 360. Typically, the roles or flow directions of port 358 and the line 360 may be reversed. That is, the needle valve 290 operates to simply open a metered passage between the port 358 and the line 360.

The shaft 354 is moved toward and away from the seat 356, thus providing a constriction between the point 352 of the shaft 354, and the seat 356. In the illustrated embodiment, a chamber 364 or passage 364 may be comparatively large or small, and provides for transition between the port 358 and the line 360.

Typically, threads 366 engage between the housing 355 and the shaft 354. Pitch of the threads is selected to provide several rotations of the shaft 354, each advancing the point 352 of the shaft 354 toward or away from the seat 356. Thereby, control is exercised over the passage way 361 that is the gap between the needle 352 and the seat 356. A seal 368 seals the shaft 354 to the housing 355 in order to prevent escape, and assure that all gas from the port 358 passes through the passage 361 into the conduit 360

A stepper 370 or stepper motor 370 may connect directly to a shaft 354. However, the stepper 370 with its own rotating shaft 372 is typically connected through a coupler 374 to be substantially collinear with the shaft 354. It has been found that the precision required in controlling the very best needle valves 290 available (which are manual) does not tolerate drive mechanisms. It has been found that driving is possible by a collinear alignment of the motor shaft 372 with the needle shaft 354. A coupler 374 may be of any various types, and may be a universal joint. In some embodiments it may simply be a fixed coupler 374. However, a fixed coupler 374 still tends to rigidize the alignment between the shafts 354, 372, and cause flexion which may lead to failure. Thus, various types of couplers 374 may be used including flexible couplers, universal couplers, or solid alignment shafts.

The stepper 370 or stepper motor 370 may be overridden by a knob 382 or manual interface 382 on the shaft 372. For example, knurling 284 on the knob 382, which is essentially a right circular cylinder, may operate to rotate the shaft 354 by means of manually rotating the shaft 372.

Typically, the stepper 370 is driven by power through lines 376 delivered from a controller 380 or control 380. The control 380 receives signals from a computer system 210, and power through a line 378. It then operates in accordance with the instructions from the computer system to rotate the stepper 370. The stepper motor 370 in one currently contemplated embodiment rotates a mere eight degrees on a needle valve 290 wherein the threads 366 have a pitch of less than a millimeter, thus advancing a needle valve 352 fifteen turns between fully opened (max flow) and fully closed. Thus, eight degrees out of a 360 degree circle and fifteen revolutions around that circle provide 675 increments, considerable precision in the passage 361 or control thereof.

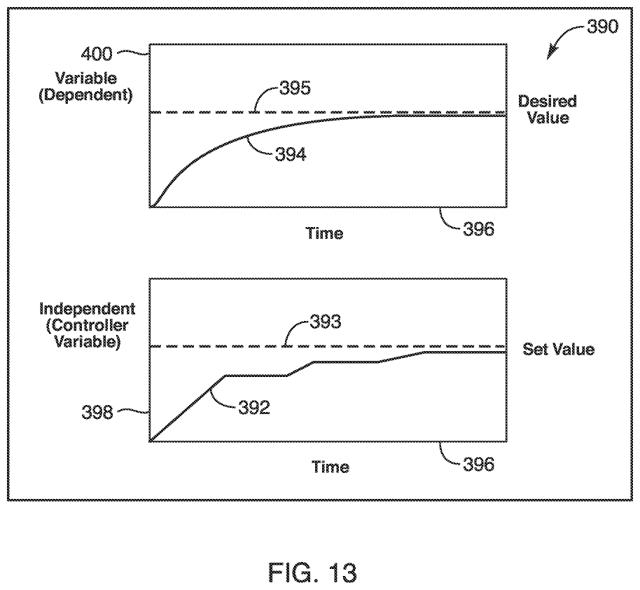

Referring to FIG. 13, the control 380 or controller 380 relies on processing by the control module 332 in the computer system 210. Lambda methods of control provide for a monotonic approach (e.g., single direction of change with no reversal) monotonically approaching a set point for the needle valve 290. This has been found to be extremely valuable and important in maintaining precision. By monotonically approaching a set point, effectively smoothly although not truly asymptotically, the precision necessary can be obtained, by avoiding any backlash.

For example, in conventional control theory, overshoot beyond a target point or set point is permitted. The control mechanism has a sufficiently high band width to draw the controlled parameter back toward the set point. For threaded needle valves and the precision of metering required here, that has not been found effective. In systems in accordance with the invention, it has been found inadequate to use conventional control theory in the available mechanical devices available today.

To control a needle valve 290 to the level of precision required by the specification of the instant invention, a chart 390 illustrates the operation of the different variables. For example, in the chart 390 or the charts 390, a curve 392 represents a value 392 of an independent variable 398. The curve 394 represents the dependent variable. For a set value 393 or set point 393 to which the needle valve 290, for example, would be positioned, the curve 392 represents the progression through a monotonic change (although shown as stepped) in that variable. Meanwhile, the set point 395 is the output or the desired dependent variable 400 that is to be set, such as flow rate.

For example, it has been found that a monotonic approach is available with no backlash using numerical methods such as predictor-corrector methods, other numerical methods that do not permit overshoot, lambda control procedures, and the like. A monotonic approach from a single side (e.g., above or below) has been shown to be extremely valuable for control of a needle valve 290 without incurring hysteresis or backlash.

In the illustrated embodiment, the curve 392 of the independent variable 398 results in a curve 394 for the dependent variable 400 that approaches a set point value 395 monotonically. Meanwhile, the time axis 396 or x axis 396 shows progress over time as the curve 394 approaches monotonically a desired value 395. Meanwhile, the value shown by the curve 392 of the independent variable 398 over a period of time 396 approaches its necessary set point 393 at whatever value obtains the result 395 for the dependent variable. That is, the set point value 393 is not really set. Rather, the value 393 becomes the point to which the curve 392 arrives by the curve 394 monotonically approaching the set point 395.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

Additional disclosure regarding systems, devices, apparatus, compositions, along with their formulation techniques, shapes, processes, and the like are disclosed in U.S. patent application Ser. No. 11/751,523, U.S. patent application Ser. No. 12/361,123, U.S. patent application Ser. No. 12/361,151, U.S. patent application Ser. No. 12/410,442, U.S. patent application Ser. No. 12/419,123, and U.S. Pat. No. 7,220,393.

According to some embodiments, a nitric oxide gas generator is provided. A nitric oxide gas generator which includes a body having a dilution inlet chamber, a chemical mixing chamber, and a dilution outlet chamber. A dilution inlet for diluent gases is provided into the dilution inlet chamber. An inlet is provided to permit entry of the diluent gases into the chemical mixing chamber. An outlet is provided to permit the exit of diluted nitric oxide gas from the chemical mixing chamber to the dilution outlet chamber. A dilution outlet is provided for removal of diluted nitric oxide gas from the dilution outlet chamber. Supports are provided for supporting chemicals to be reacted to produce nitric oxide gas. A heat source is provided to heat the chemical mixing chamber in which chemicals are mixed to initiate a chemical reaction that produces nitric oxide gas.

James D. Ray and Richard A. Ogg Jr. of the Department of Chemistry of Stanford University, developed and published a method of preparing nitric oxide in 1956. What is required is a nitric oxide generator which is capable of producing nitric oxide in accordance with the teachings of the method of Ray and Ogg Jr., and diluting the pure nitric oxide into concentrations that have utility.

According to the present invention there is provided a nitric oxide gas generator which includes a body having a dilution inlet chambers, a chemical mixing chamber, and a dilution outlet chamber. A dilution inlet for diluent gases is provided into the dilution inlet chamber. An inlet is provided to permit entry of the diluent gases into the chemical mixing chamber. An outlet is provided to permit the exit of diluted nitric oxide gas from the chemical mixing chamber to the dilution outlet chamber. A dilution outlet is provided for removal of diluted nitric oxide gas from the dilution outlet chamber. Supports are provided for supporting chemicals to be reacted to produce nitric oxide gas. A heat source is provided to heat the chemical mixing chamber in which chemicals are mixed to initiate a chemical reaction that produces nitric oxide gas.

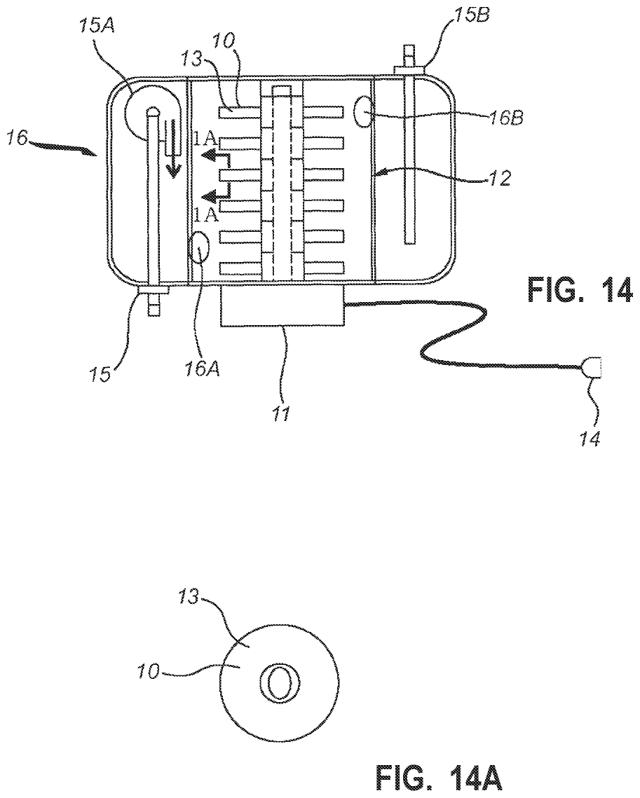

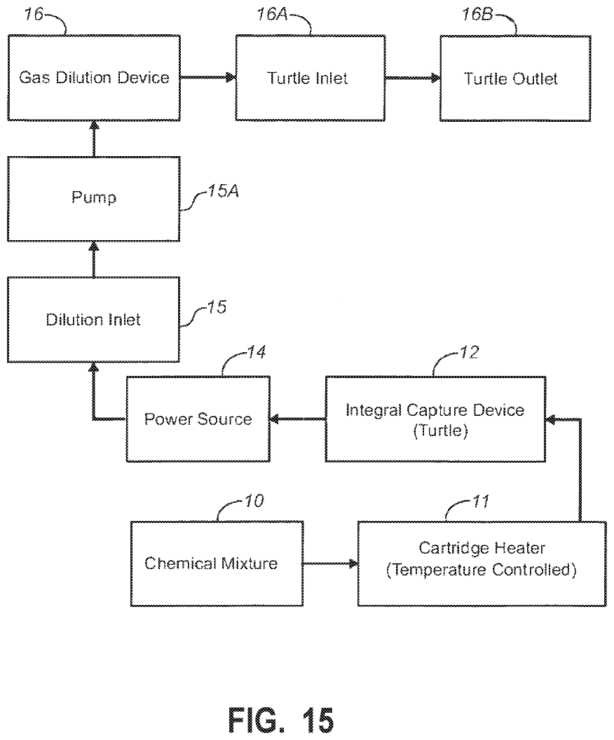

These and other features of the invention will become more apparent from the following description in which reference is made to the appended drawings, the drawings are for the purpose of illustration only and are not intended to in any way limit the scope of the invention to the particular embodiment or embodiments shown, wherein FIGS. 14-15 disclose additional advantageous aspects and features.

The preferred embodiment, a nitric oxide gas generator will now be described with reference to FIGS. 14 and 15.

### Existing Method:

(Contribution from the Department of Chemistry, Stanford University)
A New Method of Preparing Nitric Oxide
By James D. Ray and Richard A. Ogg, Jr.
Received Jul. 25, 1956

A new method of preparing nitric oxide which involves heating to a temperature slightly above 300 degrees a dry powdered mixture of potassium nitrite and nitrate, chromic oxide and ferric oxide has been perfected. The nitric oxide so produced contained only a fraction of a percent of impurity.

### 1. Description of Problem

One of the problems with the above method is the lack of accurate temperature control.

### Solution

In order to produce reliable quantities of nitric oxide gas, the temperature must be accurately controlled. We shall do this by means of an electronically controlled electric heater and by compressing the chemical mixture into a lifesaver shape, which will allow consistent repeatable heat transfer from the heater to the mixture.

### 2. Description of Problem

It is necessary to capture the nitric oxide gas as it is produced and shield it from moisture, air and other unwanted contaminants.

### Solution

An Integral heater and gas capture vessel (turtle shell) with appropriate fittings will resolve the problem. See FIG. 14.

### 3. Description of Problem

Chemical mixture inconsistency will have an adverse effect on the purity, quality, quantity and repeatability of the generated gas. Inconsistency of the generated gas is caused by settling out of the mixture components due to variations in temperature, vibration and other mechanical means.

### Solution

In order to resolve inconsistencies in the chemical mixture, the chemicals will be calcined at 950 degrees Celsius in order to remove the water of hydration and then adequately mixed and compressed into a lifesaver configuration. This will prevent separation of the chemical mixture during transportation, generation of gas, shipping and handling.

### 4. Description of Problem

The chemical mixture will only produce pure nitric oxide (one million parts per million of nitric oxide gas is generated). What is needed is a method of varying the concentration of nitric oxide gas.

### Solution

Dilution of pure nitric oxide is achieved by the entrainment of air, nitrogen, oxygen, other inert gases, or any combination thereof into the integral captured gas container. See FIG. 15.

### 5. Description of Problem

Impurities in the final product due to potassium nitrite not being of sufficient purity (contains about 10% potassium nitrate) are unacceptable.

### Solution

Obtain commercially available potassium nitrite (potassium nitrite was not available as an article of commerce in 1956).

### 6. Description of Problem

The process of creating nitric oxide as described in the 1956 method of preparation is impractical for transportation.

### Solution

Construct a self-contained generator (turtle). See FIG. 15.

### 7. Description of Problem

Control of the total Amount of Gas Produced and the Rate of Production.

### Solution

Introduction of non-reactant binding reagents, configuration (lifesaver), incremental increase of reagents of known volume (size and number of lifesavers, automatic timer for heater).

### 8. Description of Problem

Lack of Shock Resistance, Lack of Stability of Reagents to Physical Abuse

### Solution

Generator Enclosed in Turtle Shell and Lifesaver Configuration
Elements of the Nitric Oxide Gas Generator
Element 10—A Chemical Mixture
Description—The mixture is 63.75 g. (0.750 mole) potassium nitrite, 25.25 g. (0.250 mole) potassium nitrate, 76 g. (0.50 mole) chromic oxide and 120 g. (0.752 mole) ferric oxide.
Element 11—A Cartridge Heater
Description—A commercial heater capable of 310 degrees Celsius with temperature control device
Element 12—Integral Gas Capture Device (Turtle)
Description—A container that captures gas created when the chemical mixture is heated.
Element 13—A Chemical Mixture Configuration
Description—A chemical mixture is compressed into a lifesaver shape that allows convenient placement of the compressed chemical onto the heater probe.
Element 14—Power Source
Description—Commercial heater powered by an electrical outlet or a rechargeable battery (depends on the volume of gas required and the portability required).
Element 15—Plumbing and Fittings Including a Dilution Inlet
Description—Commercial plumbing and fittings used as required to direct gas to the desired location.
Element 15A—Dilution Pump
Description—A pump involving positive gas flow that enhances delivery of diluent consistently
Element 16—Gas Turtle a Inlet
Description—This device can be a compressed nitrogen cylinder or an air entrainment device such as a pump with a calibrated orifice.
Element 16B—Turtle Inlet
Description—Allows diluent into the chemical mixing chamber (turtle)
Element 16B—Dilution Outlet
Description—Provided for the removal of diluted nitric oxide gas from the dilution outlet chamber.
Element 16B—Turtle Outlet
Description—Allows diluent to exit the chemical mixing chamber (turtle).

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

It will be apparent to one skilled in the art that modifications may be made to the illustrated embodiment without departing from the spirit and scope of the invention as hereinafter defined in the claims.

According to some embodiments, a nitric oxide generator and non-deliquescent tablet for use in same is provided. A method to generate nitric oxide is disclosed in one embodiment in accordance with the invention. A tablet may be placed within a vessel such that it is in thermal communication with a heat source to receive heat therefrom. The tablet is then heated to melt at least one reactant forming the tablet. The tablet may contain reactants that are substantially non-deliquescent and form nitric oxide in response to heat from the heat source.

Although it is one of the simplest biological molecules in nature, nitric oxide plays a significant role in nearly every phase of biology and medicine. From its role as a critical endogenous regulator of blood flow and thrombosis, to a principal neurotransmitter mediating erectile function, to a major pathophysiological mediator of inflammation and host defense, there are few pathological conditions where nitric oxide does not play a significant role. Discoveries relating to nitric oxide have prompted vigorous research in a variety of fields including chemistry, molecular biology, and gene therapy. In just the last two decades, tens of thousands of scientific papers addressing various aspects of this molecule have been published, most of these within the last decade.

One method for delivering nitric oxide to the body is by inhaling therapeutic doses (e.g., 20 to 100 ppm) of nitric oxide gas. This delivery method has been introduced and studied over the last decade to treat conditions such as pulmonary hypertension, hypoxemia, respiratory distress syndrome in newborns, and sickle cell disease. Providing nitric oxide in the respiratory gas dilates pulmonary vessels by relaxing vascular smooth muscle cells. This decreases pulmonary vascular resistance and redistributes pulmonary blood flow to reduce pulmonary arterial pressure and improve arterial oxygenation.

Currently, various methods have been disclosed for generating nitric oxide, including production with polymers or electrochemical production with aqueous solutions of nitric oxide precursors. One method for producing nitric oxide was disclosed in 1956 in a paper titled “A New Method of Preparing Nitric Oxide” authored by James D. Ray and Richard A. Ogg Jr. In that paper, the authors disclosed a method for generating nitric oxide that involves heating a dry powdered mixture of potassium nitrite, potassium nitrate, chromic oxide, and ferric oxide with a yellow flame. The powder was optionally mixed with water to form a stiff paste which could be molded and dried to form cylindrical shapes or pellets. The resulting nitric oxide gas was very pure, in some cases as much as 99.78 percent pure.

Nevertheless, the composition disclosed by Ray and Ogg is not suitable to produce a stable, long-lasting tablet for generating nitric oxide. In particular, the potassium nitrite is deliquescent, tending to absorb excessive amounts of water from the atmosphere causing the material to liquefy. Other ingredients, such as the ferric oxide, are not readily compressed to form a tablet with acceptable friability and hardness.

In view of the foregoing, what is needed is a method and apparatus to produce a stable, long-lasting tablet that will release nitric oxide in suitable quantities, predictably, over a suitable time upon being heated. Further needed is an apparatus for heating and capturing nitric oxide generated by such a tablet. Further needed is an apparatus for diluting the nitric oxide to a therapeutically safe level. Yet further needed is a tablet having acceptable hardness and friability that can be manufactured in large quantities by mass production, distributed, stored, and easily used. Further needed is a tablet that will produce nitric oxide with acceptable efficiency. Yet further needed are methods, materials, and techniques to improve upon the method and composition disclosed by Ray and Ogg.

Consistent with the foregoing, and in accordance with the invention as embodied and broadly described herein, an apparatus to generate nitric oxide is disclosed in one embodiment in accordance with the invention as including a heat source and a vessel containing the heat source. A tablet may be placed within the vessel such that it is in thermal communication with the heat source to receive heat therefrom. The tablet may contain reactants that are substantially non-deliquescent and form nitric oxide in response to heat from the heat source.

In selected embodiments, the tablet further comprises an inert binder providing a substantially solid path of thermal conduction between granules of reactants. The tablet may be compressed to a hardness providing a thermal conductivity effective to heat the reactants internal thereto substantially exclusively by thermal conduction. In certain embodiments, the hardness of the tablet is selected to be greater than 5 kiloponds. In other embodiments, the hardness of the tablet is selected to be greater than 9 kiloponds. In yet other embodiments, the hardness of the tablet is selected to be from about 10 kiloponds to about 20 kiloponds.

In certain embodiments, the heat source is controlled to melt, yet avoid vaporizing, one or more of the reactants. In other embodiments, the heat source is controlled to melt one or more of the reactants, and to avoid vaporizing any of the reactants.

In certain embodiments, the reactants consist substantially of a non-deliquescent nitrite compound, a nitrate compound, and a single metal oxide. In selected embodiments, the inert binder may include calcium silicate. In other embodiments, the non-deliquescent nitrite compound may include sodium nitrite. Similarly, the nitrate compound may include potassium nitrate and the metal oxide may include chromic oxide.

In a second aspect of the invention, a stable nitric-oxide-producing tablet may include substantially non-deliquescent reactants forming nitric oxide in response to heat applied thereto. These reactants may include a non-deliquescent nitrite compound, a nitrate compound, and a metal oxide. The tablet may also include an inert binder providing a substantially solid path of thermal conduction between the reactants.

In selected embodiments, the inert binder may include calcium silicate. In other embodiments, the non-deliquescent nitrite compound may include sodium nitrite. Similarly, the nitrate compound may include potassium nitrate and the metal oxide may include chromic oxide.

In a third aspect of the invention, a method of generating nitric oxide may include providing a solid tablet comprising non-deliquescent reactants. This tablet may then be heated to melt at least one of the reactants to promote reaction thereof, thereby generating nitric oxide. The nitric oxide may then be mixed with a diluent gas to provide a therapeutically safe and effective concentration of nitric oxide.

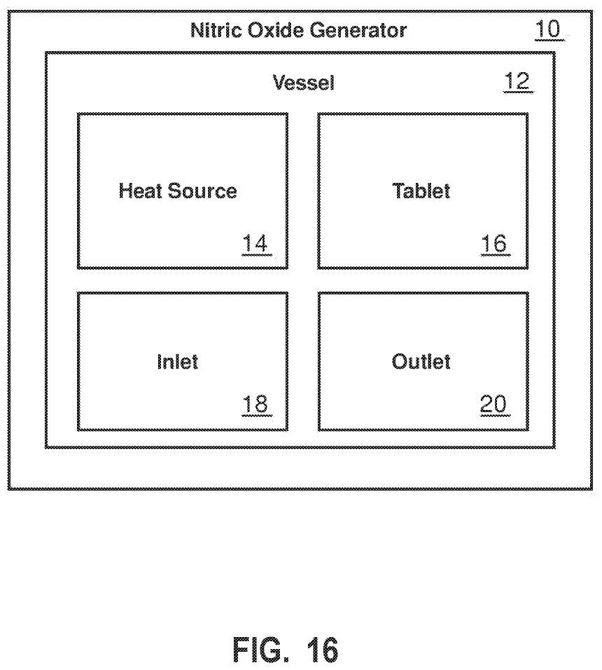

The foregoing and other objects and features of the present invention will become more fully apparent from the following description and appended claims, taken in conjunction with the accompanying drawings. Understanding that these drawings depict only typical embodiments in accordance with the invention and are, therefore, not to be considered limiting of its scope, the invention will be described with additional specificity and detail through use of the accompanying drawings in which FIGS. 16-23 disclose additional advantageous aspects and features.

It will be readily understood that the components of the present invention, as generally described and illustrated in the Figures herein, could be arranged and designed in a wide variety of different configurations. Thus, the following more detailed description of the embodiments of apparatus and methods in accordance with the present invention, as represented in the Figures, is not intended to limit the scope of the invention, as claimed, but is merely representative of certain examples of presently contemplated embodiments in accordance with the invention.

Referring to FIG. 16, in general, a nitric oxide generator 10 in accordance with the invention may include a vessel 12 and a heat source 14 within the vessel 12. The heat source 14 may be in thermal communication with a tablet 16. The tablet 16 may contain reactants that generate nitric oxide gas when heated. The reactants in the tablet 16 may be depleted after heating the tablet 16 for a given time and temperature, after which the tablet 16 may be replaced. In selected embodiments, the tablet 16 may retain its shape and structure after the reactants have been substantially depleted.

The heat source 14 may be in direct contact with the tablet 16 to conduct heat directly to the tablet 16. Alternatively, the heat source 14 may radiate heat, which may then be absorbed by the tablet 16 without physical contact. In either case, the heat source 14 may be placed inside the vessel 12 in order to efficiently transfer heat to the tablet 16. This may also provide a degree of safety when handling or coming into contact with the generator 10.

In selected embodiments, the nitric oxide generator 10 may also include an inlet 18 to allow diluent gases to enter the vessel 12 and thereby mix with and dilute the nitric oxide gas. The resulting diluted nitric oxide gas may then exit the vessel 12 through an outlet 20 where it may be stored or conveyed to a person or animal to provide therapy.

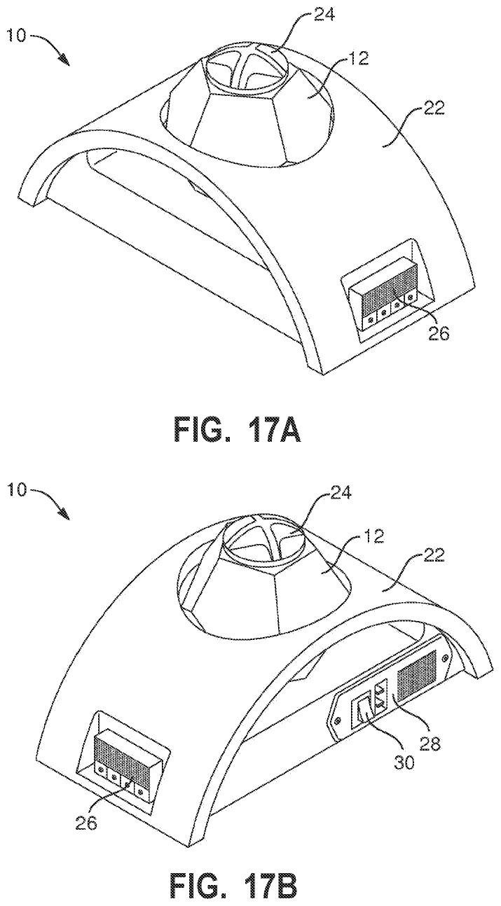

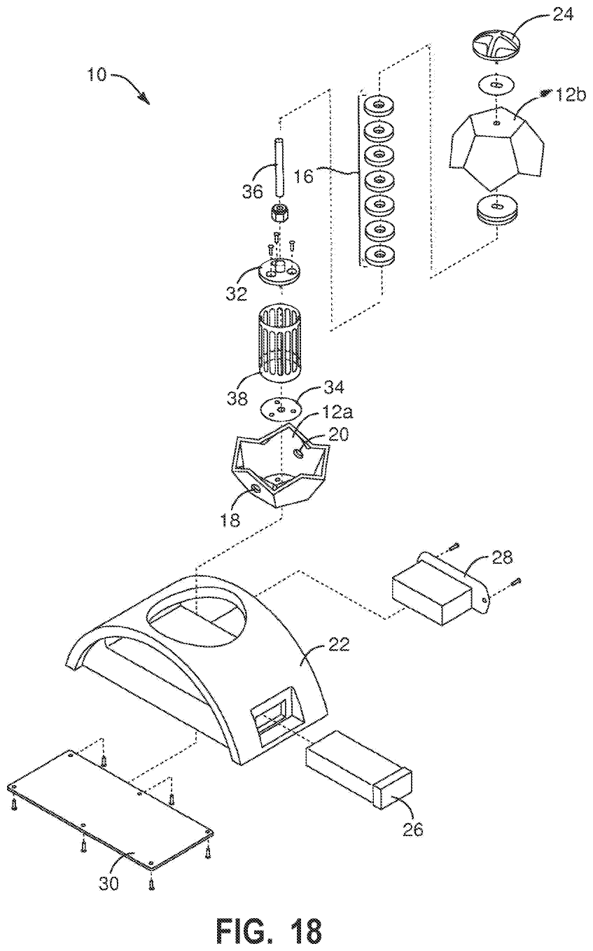

Referring to FIGS. 17A and 17B, one specific and non-limiting implementation of a nitric oxide generator 10 in accordance with the invention is illustrated. As shown, in one embodiment a nitric oxide generator 10 may include a body portion 22 which may support a vessel 12. In this embodiment, the vessel 12 is provided in the form of a clam shell which may include separable upper and lower portions. These portions may be separated to provide access to the heat source 14 and allow tablets 16 to be inserted or replaced. In selected embodiments, a thumbscrew 24 or clamp 24 may be used to keep the upper and lower portions of the clam shell together to seal the vessel 12 and prevent injury. As will be shown in FIG. 18, the clam shell may also include one or more inlets 18 and outlets 20 to allow diluent gases to enter the vessel 12, mix with nitric oxide gas, and exit the vessel 12. These inlets 18 and outlets 20 may, in certain embodiments, be fitted with a latch, coupling, fitting, or other connector to connect to a hose or other device.

In selected embodiments, a generator 10 may also include a control panel 26 to adjust various operational parameters of the nitric oxide generator 10. For example, the control panel 26 may be used to adjust the temperature, current, or heat output of the heat source 14 which may in turn adjust the amount of nitric oxide produced. This may be used to adjust the concentration of nitric oxide in gases exiting the vessel 12. In other embodiments, the control panel 26 may be used to regulate the flow of diluent gases through the vessel 12 with a valve or other control device. This may also adjust the concentration of nitric oxide in gases exiting the vessel 12. In other embodiments, the control panel 26 may be used to turn the generator 10 on or off, set a timer to turn the generator 10 on or off, set a temperature, current, or heat profile for the heat source 14 that changes (e.g. monotonically or programmatically) over time 14, or the like. Similarly, the control panel 26 may be configured to trigger one or more alarms when the nitric oxide concentration rises above or falls below a selected threshold. These examples represent just a few possible functions for a control panel 26.

The generator 10 may also include a power supply panel 28 connecting to a power cord or other source of electricity. A switch 30 may be provided to selectively connect or interrupt the supply of power to the generator 10.

Referring to FIG. 18, an exploded view of the generator 10 of FIGS. 17A and 17B is illustrated. As shown, the generator 10 may include a body portion 22, a control panel 26, and power supply panel 28, which may be inserted into the body portion 22. A cover plate 30 may be used to provide access to the control panel 26, power supply 28, and possibly a heating element 36 from below the generator 10. A vessel 12, in this example a selectively opened and closed clam shell 12 a, 12 b, may be inserted into or mounted to the body portion 22. A lower portion 12a of the vessel 12 may be attached to the body portion 22 using a mounting plate 32, washer 34, and one or more fasteners, such as screws, rivets, welds, or the like. The lower portion 12 a may include one or several ports, such as an inlet 18 and outlet 20, to allow diluent gases to pass through the vessel 12.

In selected embodiments, a heating element 36, such as a calrod 36 or cartridge heater 36, may be inserted through the mounting plate 32 and washer 34 where it may be connected to a power source outside the vessel 12 a, 12 b. One or more tablets 16, having apertures therein, may be placed over the heating element 36. The tablets 16 may be stacked directly on top of one another or may be separated by a washer or other spacer. The tablets 16 may be heated through direct contact with the heating element 36 or may, alternatively, absorb heat radiated from the heating element 36. The temperature of the heating element 36 may be controlled to provide a regulated amount of heat (e.g., between 200° C. and 700° C.) to the tablets 16. This enables nitric oxide to be generated over a period of time and in a controlled manner.

In selected embodiments, the tablets 16 may be surrounded by a perforated baffle 38. The baffle 38 may regulate heat dissipation from the tablets 16, provide more uniform heating of the tablets 16, regulate the flow of diluent gases over the tablets 16, or the like. The baffle 38, by contrast, may allow nitric oxide gas to pass through slots or apertures in the baffle 38 to mix with diluent gases passing through the vessel 12.

An upper portion 12 b of the vessel may be used to cover the heating element 36 and tablets 16, seal the vessel 12, prevent the escape of nitric oxide, and retain heat within the vessel 12. The upper portion 12 b may be retained over the lower portion 12 a by, for example, a thumbscrew 24, clamp 24, or other suitable retention mechanism.

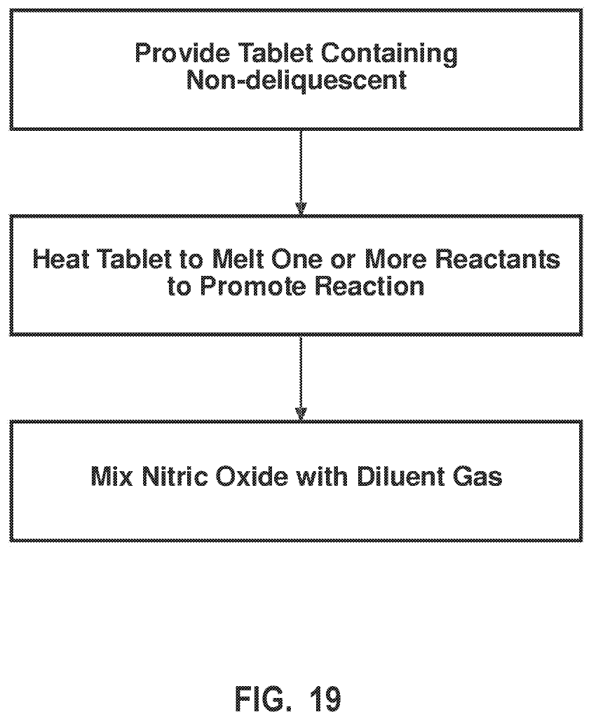

Referring to FIG. 19, in selected embodiments, a method 50 for generating nitric oxide may include providing 52 one or more tablets 16 containing non-deliquescent reactants for producing nitric oxide. Use of non-deliquescent reactants enables manufacture of an environmentally stable tablet 16 that will retain its ability to produce nitric oxide over time. As will be explained in more detail hereafter, the stability of the reactants enable production of a tablet 16 that is both harder and less friable than may be possible with deliquescent reactants. Additional hardness, which may be a function of the amount of compressive force applied to the tablet 16, may increase the nitric oxide production of the tablet 16 by improving the solidity and thus thermal conductivity of the tablet 16. As will be explained in more detail hereafter, this provides a tablet 16 having improved stability and an improved ability to produce nitric oxide, and to do so more predictably, compared to a tablet 16 containing one or more deliquescent reactants, such as potassium nitrite.

Once a tablet 16 is provided 52, the method may include heating 54 the tablet 16 to melt one or more reactants. This may cause the melted reactants to come into intimate molecular contact, and even to migrate through the tablet until they come into contact with other reactants, thereby initiating the nitric-oxide producing reaction. This also enables certain ones of the reactants to be reacted as liquids (or vapors, or both) with heat of a fairly modest temperature (e.g., 300-500° C.). In certain embodiments, reactants may be vaporized to react in a vapor phase. In selected embodiments, the substantially non-deliquescent reactants may include sodium nitrite, potassium nitrate, and chromium oxide. These reactants may produce nitric oxide in accordance with the following stoichiometric equation:

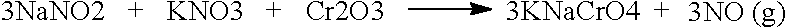

Of the above reactants, sodium nitrite (NaNO2) has the lowest melting temperature (270° C.). Thus, upon heating the reactants to 270° C., the sodium nitrite may begin to melt and intermingle with molecules of other reactants. It may even flow through the tablet 16 to make intimate contact with other reactants, thereby initiating the nitric-oxide-producing reaction. In selected embodiments, the temperature may be controlled to avoid vaporizing any of the reactants. Thus, the reaction may occur mostly in the solid and liquid phases of reactants.

For example, sodium nitrite has the lowest boiling point (320° C.) of the reactants. Thus, in certain embodiments the temperature of the heating element 36 may be maintained between about 270° C. and 320° C. to melt the sodium nitrite while avoiding vaporizing it. Thus, a liquid reactant can move to contact solid reactants. By controlling the temperature, the reaction may be controlled to allow the nitric oxide to be released over a desired period of time, such as, for example, about 30 minutes. Nevertheless, in other embodiments, the reactants may be heated to greater temperatures, such as between about 300° C. and 700° C. Thus, although the generator 10 may generate nitric oxide at lower temperatures, its use is not limited to the lower temperatures.

Once the reaction is generating nitric oxide, the resulting nitric oxide gas may be mixed 56 with a diluent gas, such as nitrogen, air, or the like, to dilute the nitric oxide to a therapeutically safe level, such as between about 20 and about 500 ppm. A range of about 250 to about 400 ppm is particularly useful, with a target of just over 300 ppm. In selected embodiments, the diluent gas may be pumped into the vessel 12 at a desired rate (e.g., 0.5 L/min) where it may mix with the nitric oxide and exit through an outlet 20. In other embodiments, the nitric oxide may be drawn into a stream of diluent gas using a principle such as the venturi effect.

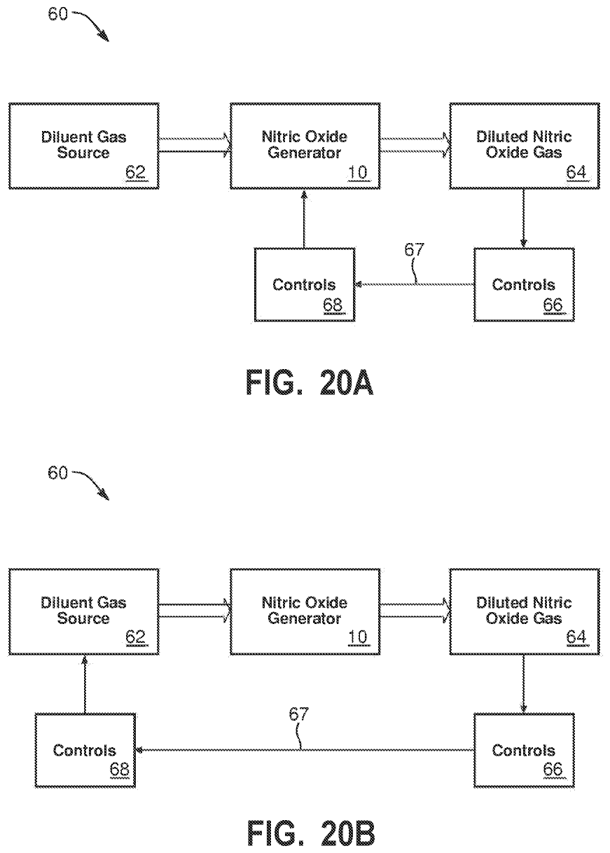

Referring to FIG. 20A, in selected embodiments, a nitric oxide generation system 60 may include a diluent gas source 62, a nitric oxide generator 10, and a destination for diluted nitric oxide gas 64. In certain embodiments, the system 60 may utilize a feedback loop to control the concentration of nitric oxide gas in the diluted nitric oxide gas 64. For example, one or more sensors 66 may be used to sense the concentration of nitric oxide in the diluted nitric oxide gas 64. These sensors may provide a feedback signal 67 to various controls 68. These controls 68 may be used to adjust the temperature of a heating element 36 of the nitric oxide generator 10. By adjusting the temperature, heat, current, or other energy control point, the speed of the reaction and thus the nitric oxide release rate may be adjusted to achieve a desired concentration of nitric oxide in the diluted gas 64.

Referring to FIG. 20B, in an alternative embodiment, a feedback signal 67 may be used to control the flow rate of a diluent gas through the nitric oxide generator 10. This may also adjust the concentration of nitric oxide in the diluted gas 64. This may be accomplished, for example, by adjusting the speed of a pump moving the diluent gas. Alternatively, the feedback signal may be used to control a valve to regulate the flow rate of diluent gases through the nitric oxide generator 10. In selected embodiments, a system 60 may utilize both types of feedback illustrated in FIGS. 20A and 20B to control the concentration of nitric oxide in the stream or supply of diluted gas 64.

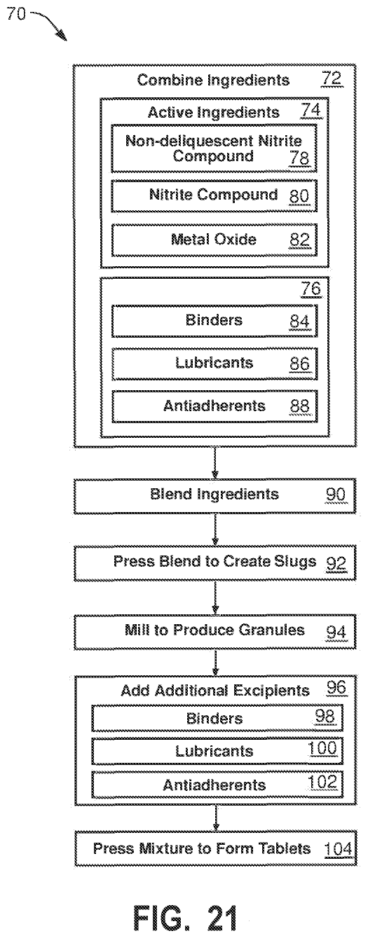

Referring to FIG. 21, one embodiment of a method 70 for making a nitric-oxide-producing tablet 16 using a dry granulation process is illustrated. In certain embodiments, such a method 70 may include initially combining 72 various ingredients, such as active ingredients 74 and excipients 76, to produce a tablet 16. In selected embodiments, active ingredients may include a non-deliquescent nitrite compound 78, a nitrate compound 80, and a metal oxide 82. In one embodiment, the active ingredients may include about 33 percent by weight of sodium nitrite, about 17 percent by weight of potassium nitrate, and about 50 percent by weight of chromic oxide. A stoichiometric mixture may be used or an excess of all ingredients except for a rate controlling reactant.

The tablet 16 may also include one or more excipients 76 that may improve the manufacturability of the tablet 16 as well as increase the thermal conductivity, heat transfer capacity, or temperature uniformity, and thus nitric oxide production, of the tablet 16. For example, the tablet 16 may include one or more binders 84, lubricants 86, and antiadherents 88. In one embodiment, a suitable binder 84 may include calcium silicate (Ca2SiO4), a suitable lubricant 86 may include zinc stearate, and a suitable antiadherent 88 may include talc to prevent punch sticking. The calcium silicate acts a compression aid to produce a tablet 16 with acceptable hardness and friability. The calcium silicate does not replace the ferric oxide disclosed by Ray and Ogg. It serves a different function without harming nitric oxide production. In fact, ferric oxide was found to be detrimental to tablet 16 physical properties, producing tablets 16 with unacceptable brittleness and friability.

In selected embodiments, combining 72 the ingredients may include initially combining all the active ingredients 74 with about half of the excipients 76. These ingredients may then be blended 90 with a device such as a V-blender. This mixture may then be pressed 92 using a tablet or other suitable press to create slugs containing the above-mentioned ingredients. In selected embodiments, the slugs may be pressed to a hardness above about 5 kiloponds. In other embodiments, the slugs may be pressed to a hardness of between about 10 and 20 kiloponds. In other embodiments, the slugs may be pressed to a hardness of about 14 kiloponds.

The compressive force applied to the tablets 16 may be important and may affect the nitric oxide production of the tablets 16. In general, a higher compressive force will improve the nitric oxide production of a tablet 16. Higher compressive forces reduce air volume and improve chemical intimacy between the reactants, as well as increasing the thermal conductivity of the tablet 16 by both conforming particles to one another and removing pores or other voids in the tablet 16. The improved thermal conductivity provides better heat transfer to the reactants, and better molecular contact, thereby providing more uniform heating and better nitric oxide production.

Once created, the slugs may be milled 94, such as with a Fitzmill Model DASO 6, to produce granules. These granules may be filtered through, for example, about a number 20 mesh screen to remove larger particles. The granules, as well as the remaining binder 98, lubricant 100, and antiadherents 102 may then be combined 96 and returned to the blender for mixing. This mixture may then be returned to the tablet press to create 104 the tablets 16. In selected embodiments, a different tool or die may be used to produce tablets 16 with an aperture in the middle, as illustrated in FIG. 3. In certain embodiments, the tablets 16 may be pressed to a final hardness above about 5 kiloponds. In other embodiments, the tablets 16 may be pressed to a hardness of between about 10 and 20 kiloponds. In other embodiments, the tablets 16 may be pressed to a hardness of about 17 kiloponds.

Tablets 16 made in accordance with a method 70 have been found to have greatly improved physical properties. They also exhibit significantly improved nitric oxide production in the generator 10 disclosed by Applicants. That is, the tablets 16 greatly outperform the powders, “pellets,” or molded “cylindrical pieces” disclosed by Ray and Ogg when heated in the nitric oxide generator 10. Because of the improved performance, significantly less amounts of active ingredients are required to produce a tablet 16 having acceptable nitric oxide production.

For example, a 5 gram tablet made in accordance with Ray and Ogg's method and containing approximately 85 percent by weight of active ingredients produced only 2.4 mL of nitric oxide gas when heated in the generator 10. By contrast, a five gram tablet 10 made in accordance with a method 70 and containing only 10 percent by weight of active ingredients produced about 11.5 mL of nitric oxide in the generator 10. This constitutes a more than 3000 percent increase in efficiency.

In selected embodiments, a tablet 16 made in accordance with the method 70 and exhibiting vastly improved efficiency may include about 3.3 percent by weight of sodium nitrite, about 1.7 percent by weight of potassium nitrate, about 5 percent by weight of chromic oxide, about 87 percent by weight of calcium silicate, about 2 percent by weight of zinc stearate, and about 1 percent by weight of talc. When compressed to a hardness of about 12.9 kiloponds, a 5 gram tablet 16 having the above composition produced approximately 11.5 mL of nitric oxide and had acceptable friability to create a satisfactory manufactured product.

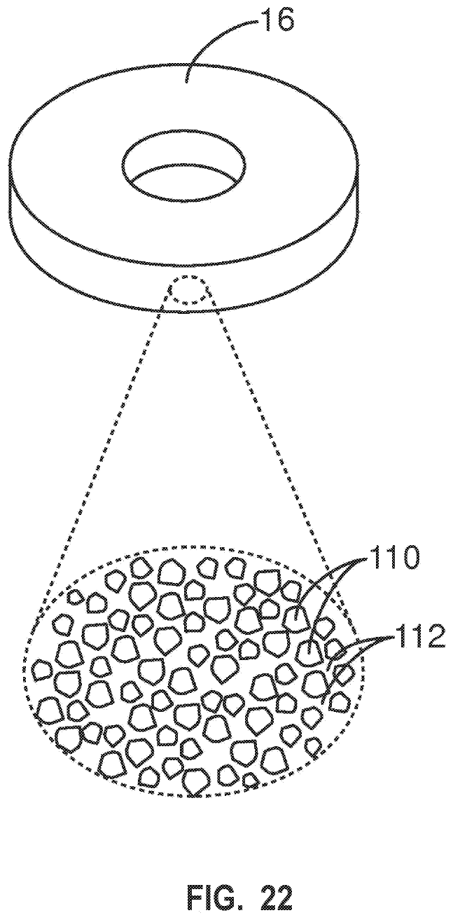

Referring to FIG. 22, as mentioned, a tablet 16 in accordance with the invention may be provided as a granulated structure that may actually increase nitric oxide production. For example, as illustrated, reactants within the tablet 16 may be agglomerated into granulated subdomains 110 within the tablet 16. The binder 112 (e.g., calcium silicate) may be present within and between the granules 110. Each of the subdomains 110 may be compressed to a hardness of greater than 5 kiloponds and more ideally between about 10 and 20 kiloponds to create chemical intimacy between the reactants of each granule. As was described in association with FIG. 21, this may be accomplished by creating slugs from a mixture containing the reactants and then milling the slugs to create granules 110 of a desired size. These granules 110 may also improve the flowability of the mixture to prevent the mixture from sticking together, thereby enabling production of a tablet 16 with improved physical characteristics.

As mentioned, the calcium silicate binder is a material that acts as a compression aid when forming the tablet 16. An unexpected benefit provided by the calcium silicate after compression is that it provides an effective path of thermal conduction to the reactants. This path of thermal conduction is provided both intergranularly, by the binder included within each granule 110, as well as extra-granularly, by the binder 112 provided between each granule 110. These paths of thermal conduction provide an effective mechanism to transfer heat to each granule 110 and to the reactants within each granule 110. This enables heat to be more efficiently transported to the reactants, significantly improving nitric oxide production by getting more of the reactants to react with one another. It follows that greater compressive forces applied to the tablet 16 may actually increase the chemical intimacy between the reactants and the binder and thus improve nitric oxide production.

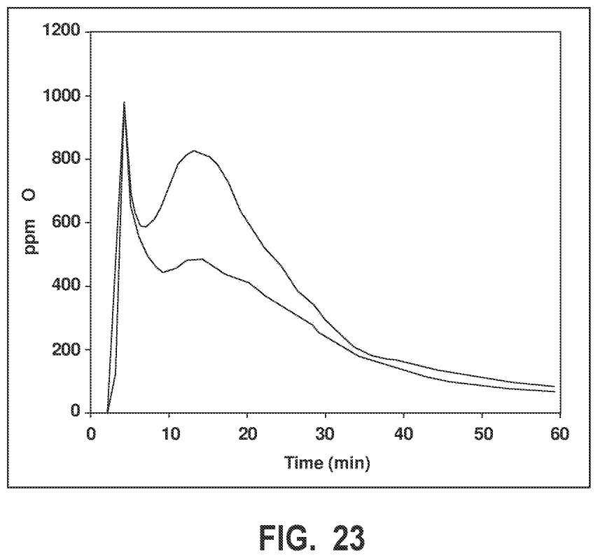

FIG. 23 is a graph showing the effect of compressive force on nitric oxide production. Each of the curves 120 a, 120 b represents the nitric oxide production of one 5-gram tablet 16 containing 10 percent by weight active ingredients and made in accordance with the method 70 illustrated in FIG. 21. The tablets 16 were heated to about 500° C. in the nitric oxide generator 10 illustrated in FIGS. 17A and 17B with nitrogen gas passing through the unit at a flow rate of about 0.5 L/min. The nitric oxide concentration was measured (in ppm) in the outgoing gas stream, as shown on the vertical axis of the graph.

Both tablets 16 represented by the curves 120 a, 120 b contain about 3.3 percent by weight of sodium nitrite, about 1.7 percent by weight of potassium nitrate, about 5 percent by weight of chromic oxide, about 89 percent by weight of calcium silicate, and about 1 percent by weight of zinc stearate. The only significant difference between the tablets 16 is that the tablet 16 represented by the curve 120 a was compressed to a hardness of about 16.0 kiloponds, whereas the tablet 16 represented by the curve 120 b was compressed to a hardness of about 10.0 kiloponds.

As can be seen for both tablets 16, nitric oxide production is greatest at the beginning of production. This production diminishes over the typical (e.g. about 30 to 90 minutes) 60 minute interval during which the reactants are consumed. As can also be observed, the tablet 16 represented by the curve 120 a, which was compressed to a hardness of about 16.0 kiloponds, generated significantly more nitric oxide than the tablet 16 represented by the curve 120 b and compressed to a hardness of about 10.0 kiloponds. These results bolster the conclusion that greater compressive forces applied to the tablet 16 increase the thermal conductivity, chemical intimacy, or both of the tablets 16 and thus improve nitric oxide production. That is, greater compressive forces achieve nitric oxide yields, in a stream of a breathing gas (e.g. air, nitrogen) having a volume of about half a liter per minute, closer to the theoretical yield. These results also achieve a target yield of at least 300 ppm of nitric oxide for at least 30 minutes.

The present invention may be embodied in other specific forms without departing from its basic features or essential characteristics. The described embodiments are to be considered in all respects only as illustrative, and not restrictive. The scope of the invention is, therefore, indicated by the appended claims, rather than by the foregoing description. All changes within the meaning and range of equivalency of the claims are to be embraced within their scope.

According to some embodiments, a portable nitric oxide generator is provided. An apparatus for portable delivery of nitric oxide without the need for pressurized tanks, power supplies, or other devices provides a single therapy session by triggering a heater to heat a reaction chamber. A piercing assembly may trigger to open sealed containers, such as bags, of liquid water or salt water in order to activate the heaters. Upon addition of liquid such as water or salt water to a chemically reactive heating element, heat is generated to activate the chemicals generating nitric oxide within a sealed reactor. Upon triggering, liquid containers are unsealed, the liquid drains down to initiate reaction of the heating chemicals, and the heat begins to penetrate the reactor. The reactor, in turn, heats its contents, which react to form nitric oxide expelled by the reactor to a line feeding a cannula for therapy.

The discovery of certain nitric oxide effects in live tissue garnered a Nobel prize. Much of the work in determining the mechanisms for implementing and the effects of nitric oxide administration are reported in literature. In its application however, introduction of bottled nitric oxide to the human body has traditionally been extremely expensive. The therapies, compositions, and preparations are sufficiently expensive to inhibit more widespread use of such therapies. What is needed is a comparatively inexpensive mechanism for introducing nitric oxide in a single dosage over a predetermined period of time. Also, what is needed is a simple introduction method for providing nitric oxide suitable for inhaling.

It would be an advance in the art to provide a single dose generator suitable for administration of nitric oxide gas. It would be an advance in the art to provide not only an independence from bottled gas, but from the need for a source of power for heat, or the like. It would be a further advance in the art to provide a disposable generator to be initiated by a trigger mechanism and operate without further supervision, adjustment, management, or the like. Likewise, it would be a substantial benefit to provide a system that requires a minimum of knowledge or understanding of the system, which might still be safe for an individual user to administer with or without professional supervision.

In accordance with the foregoing, certain embodiments of an apparatus and method in accordance with the invention provide a self-contained reactor system. Nitric oxide may thus be introduced into the breathing air of a subject. Nitric oxide amounts may be engineered to deliver a therapeutically effective amount on the order of a comparatively low hundreds of parts per million, or in thousands of parts per million. For example, sufficient nitric oxide may be presented through nasal inhalation to provide approximately five thousand parts per million in breathing air. This may be diluted due to additional bypass breathing through nasal inhalation or through oral inhalation.

One embodiment of an apparatus and method in accordance with the present invention may rely on a small reactor. Reactive solids may be appropriately combined dry. Reactants may include nitrite compounds, such as potassium nitrite, sodium nitrite, or the like, nitrate compounds, such as potassium nitrate, sodium nitrate, or the like. The reaction may begin upon introduction of a heat. Heat may be initiated by liquid transport material to support ionic or other chemical reaction in a heat device.

An apparatus and method in accordance with the invention may include an insulating structure, shaped in a convenient configuration such as a rectangular box, a cylindrical container, or the like. The insulating container may be sealed either inside or out with a containment vessel to prevent leakage of liquids therefrom. Such a system need not be constructed to sustain nor contain pressure. Inside the containment vessel may be positioned heating elements such as those commercially available as chemical heaters.

In certain embodiments, chemical heaters may include metals finely divided to readily react with oxygen or solid oxidizers. Various other chemical compositions of modest reactivity may be used to generate heat readily without the need for a flame, electrical power, or the like.

Above the heating element or heater within the containment vessel may be located a reactor. The reactor may preferably contain a chemically stable composition for generating nitric oxide. Such compositions, along with their formulation techniques, shapes, processes, and the like are disclosed in U.S. patent application Ser. No. 11/751,523 and U.S. Pat. No. 7,220,393, both incorporated herein by reference in their entireties as to all that they teach.

The reactor may include any composition suitable for generating nitric oxide by the activation available from heat. The reactor may be substantially sealed except for an outlet, such as a tubular member secured thereto to seal a path for exit of nitric oxide from the reactor.

In certain embodiments, a system of water or salt water may be available in the container. In one embodiment, the water containers may be as simple as presealed bags, such as polyethylene bags that can be opened, cut, torn, or otherwise pierced in order to release water therefrom. Accordingly, a system may include a heating element or the reactor, such a water source to provide a chemical transport fluid, a piercing assembly for the water containers, a trigger for activating the piercing assembly, and blades, hooks, cutters, punches, or the like structured to open the bags containing water.

Upon triggering of the piercing assembly, the water is released from the water containers, vessels, bags, or the like, to be poured down through the assembly onto the heating elements where heaters are activated by the presence of a liquid. It has been found through experiments that adding the additional ionic content of salt improves the reaction rate of chemical heating systems.

Ultimately, an apparatus in accordance with the invention may include a cover through which an outlet penetrates from the reactor in order to connect to a cannula. This has been done effectively. It will also support a vent for steam generated by the heaters in the presence of the water used to activate the heaters. The system may be completely wrapped in a pre-packaged assembly. In one embodiment, a heat-shrinkable wrapping material may be used to seal the outer container of an apparatus in accordance with the invention. Thus, this system may be rendered tamper proof, while also being maintained in integral condition throughout its distribution, storage, and use.

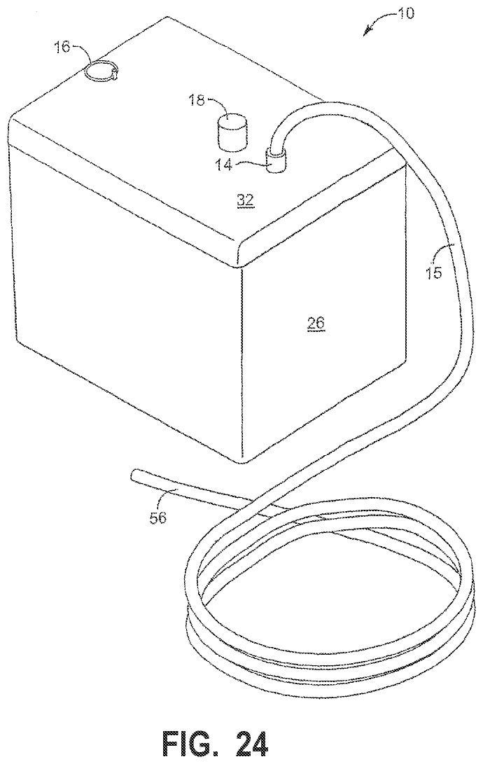

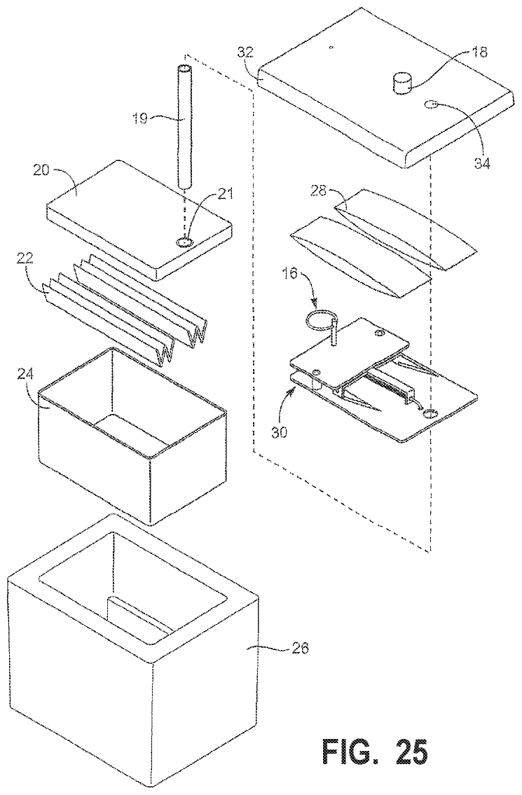

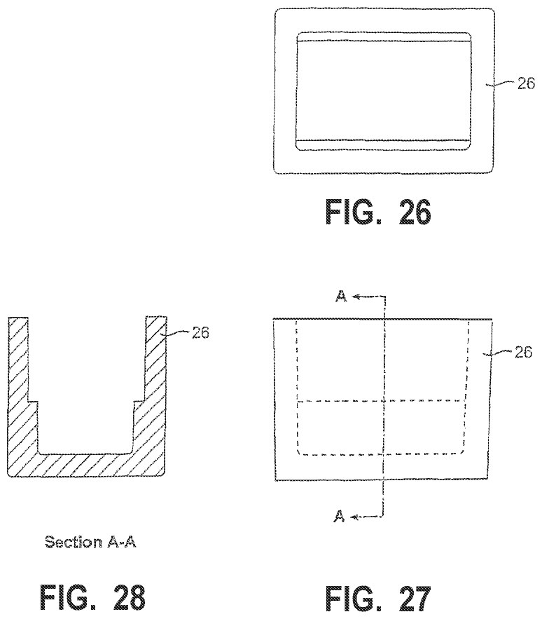

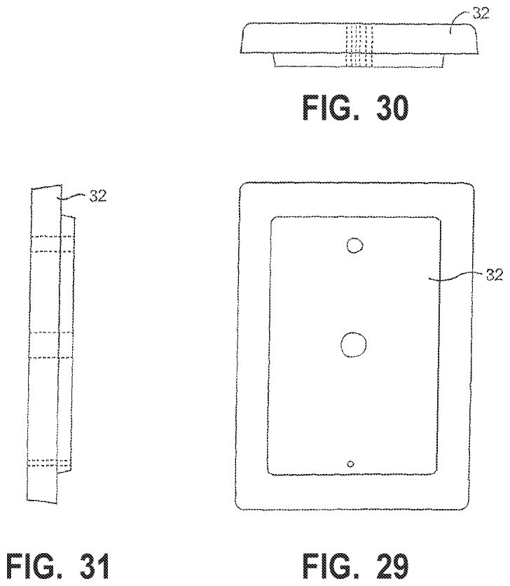

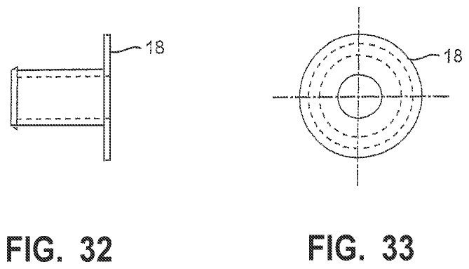

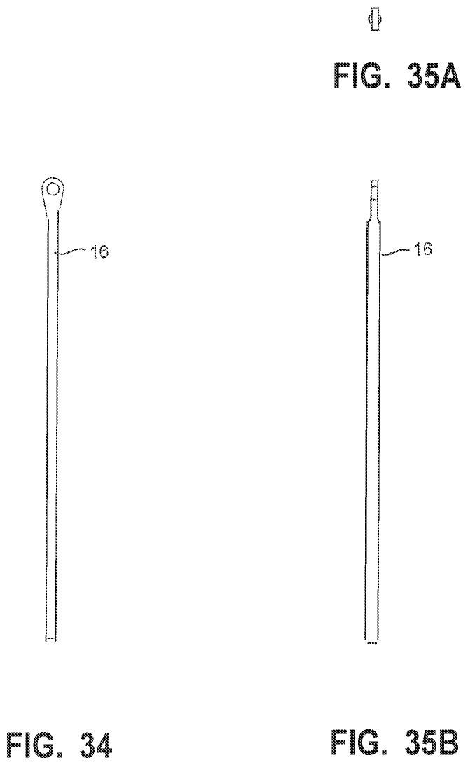

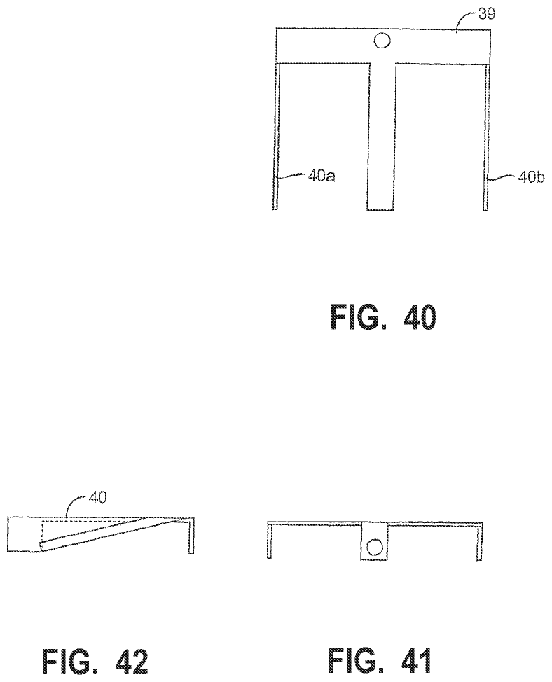

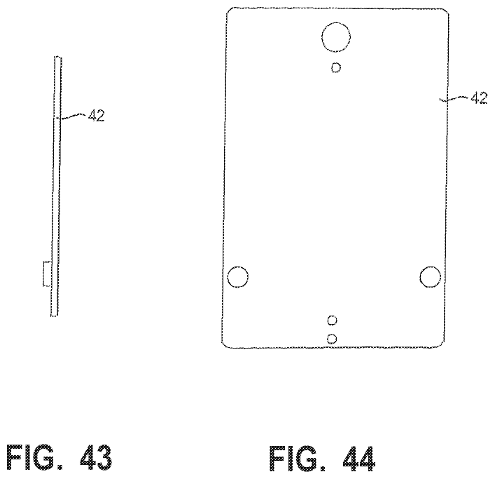

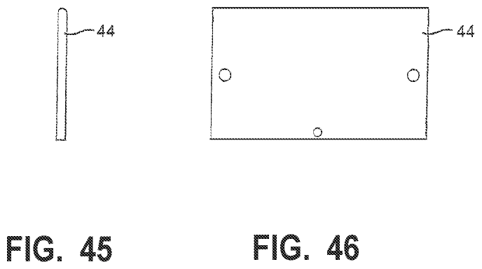

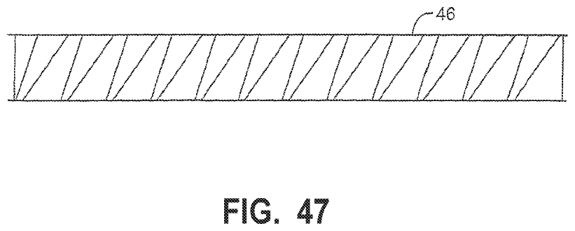

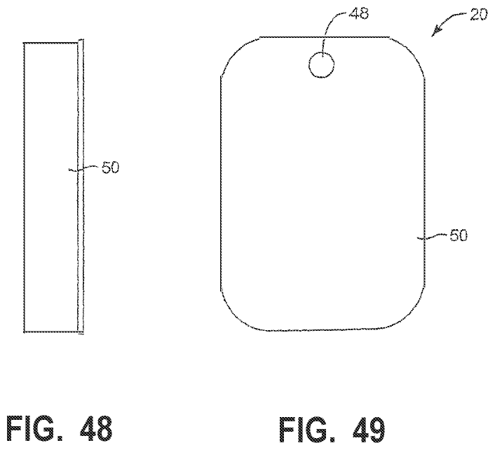

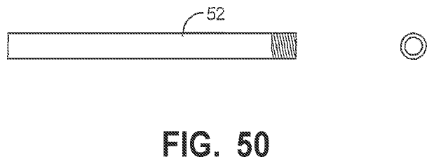

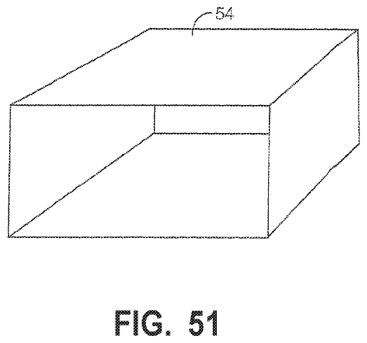

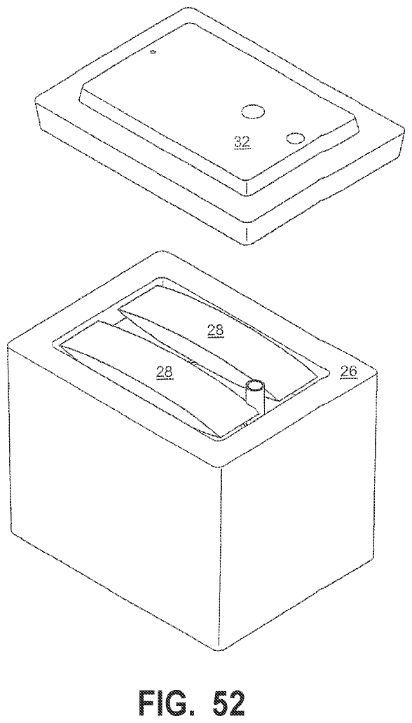

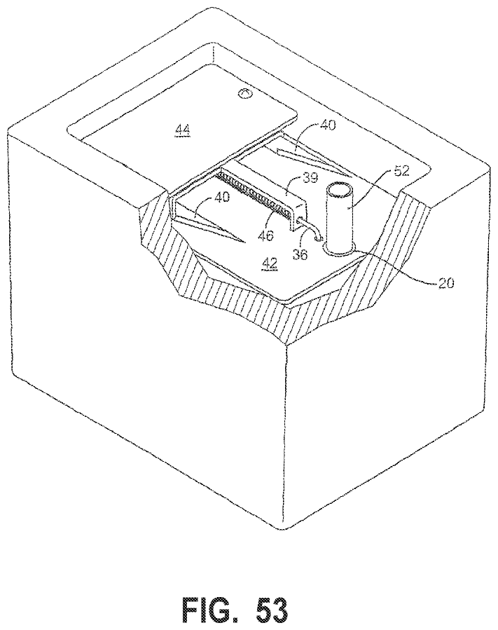

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which FIGS. 24-57 disclose additional advantageous aspects and features.

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention.

Referring to FIG. 24, an apparatus 10 may be configured as a portable nitric oxide device. In the illustrated embodiment, a container 12 or vessel 12 may provide insulation, liquid sealing, or both. Meanwhile, a fitting 14 or outlet 14 may be connected to feed nitric oxide to a line 15 proceeding toward a user, for distribution by a cannula, mask, tent, or the like.

In the illustrated embodiment, a trigger 16 or actuator 16 may be withdrawn from the apparatus 10 in order to trigger the initiation of a reaction generating nitric oxide. In certain embodiments, generation of nitric oxide may depend on temperature of reactants. The generation of heat (e.g., temperature) may rely on a reaction requiring moisture, which moisture may eventually be partially converted to steam needing to be vented. Accordingly, a vent 18 may vent the interior of the container 12 in order to avoid any buildup of pressure; in one embodiment, the entire container 12 may be sealed in a heat-shrinkable sleeve that maintains the integrity of the apparatus 10 during distribution, storage, and use.

Referring to FIG. 25, an exploded view of the apparatus of FIG. 24 illustrates one embodiment of the apparatus 10 in accordance with the invention. In the illustrated embodiment, the outlet 19, connected to feed through the fitting 14 and thus feed nitric oxide through the line 15 may be securely sealed to a reactor 20. The reactor 20 may be formed by any of several suitable methods to contain the chemical constituents required to generate nitric oxide. A port 21 or aperture 21 may be formed to seal against the outlet 19 in order to discharge all of the generated nitric oxide to a location outside the apparatus 10.

Below or around the reactor 20 may be located one or more heaters 22 or heating elements 22. In the illustrated embodiment, the heaters 22 are formed to contain solid reactants in a non-woven fabric container. The reactants are stabilized by being completely dry. In the presence of liquid, ionic exchange promotes the reaction of the contained chemicals within the heaters 22.

In order to contain any liquid to activate the heaters 22, a containment vessel 24 may surround the heaters 22, within the insulation container 26 or box 26. In certain embodiments, the functionality of the containment vessel 24 and the insulated container 26 may be consolidated into a single structure. Likewise, in certain embodiments, the containment vessel 24 may actually be located external to the insulated container 26.

In general, a liquid, and particularly a hydrating liquid such as water, salt water, or the like, may serve as an activation material. In the illustrated embodiment, the bags 28 containing salt water, water, or the like may be sealed for storage. In certain embodiments, the containers 28 may be capped, vented, or otherwise made resealable. However, in other embodiments, a fully disposable apparatus 10 may rely on inexpensive materials such as polyethylene film to form the containers 28.

By any means, an opening assembly 30 (in the illustrated embodiment, a piercing assembly 30) may be actuated to open, pierce, or otherwise breach the sealing of the containers 28 of liquid. Upon piercing or otherwise breaching of the integrity of the containers 28, the contained liquid then flows downward to be absorbed within the covering material of the heaters 22. The presence of the liquid activates the chemical reactions within the heaters 22, generating heat to initiate reaction of the chemical constituents contained within the reactor 20.

A cover 32 may enclose the insulated container 26, and may typically be formed of the same material. A vent 30 may vent steam from within the containment vessel 24 and the insulated container 26 in order to alleviate any pressure build up. Likewise, in order to direct the residual steam in a specific direction other than permitting it to escape about the interface between the cover 32 and the container 26, a vent 18 may be advisable, required, or otherwise useful.

The outlet 19 for nitric oxide may penetrate through the cover 32 by means of an aperture 34. The aperture 34 may be sealed against the outlet 19 in order that the steam generated from the heaters 22 escape substantially exclusively through the vent 18, rather than near the fitting 14 and line 15 that may be subject to manipulation by the user.

Referring to 3-8 and 29, the insulated container 26 may be formed in any suitable shape to contain all of the elements required for a single dosing of nitric oxide. Accordingly, the constituent structures of FIG. 25 may fit within the interior of the container 26. Meanwhile, cover 32 may be fitted thereto.

The vent 18 may be formed to fit snugly through a penetration in the cover 32. A flange thereof may be labeled with colors and text appropriate to warn of the elevated temperature thereof as a safety measure.

A pin may act as a significant portion of the trigger assembly 16 or trigger 16. Upon removal of the pin, such as by a user pulling on a handle or ring secured thereto, the blades may be released to pierce the containers 28 holding the liquid required to initiate the reaction of heaters 22.

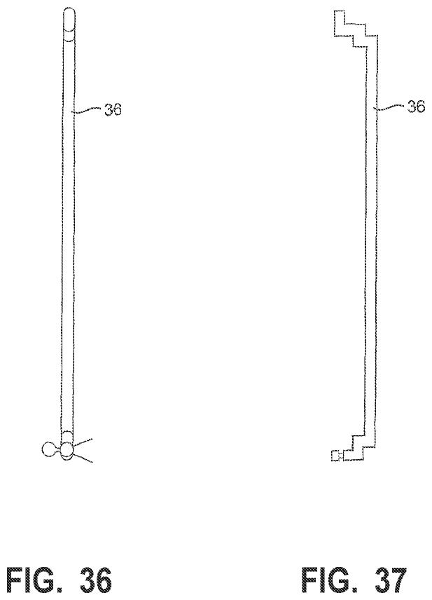

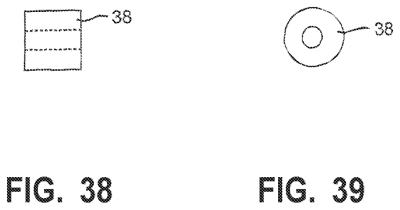

A guide 36 or guide rod 36 may direct the blades of the piercing assembly 30. A compression spring wrapped around the guide 36 or rod 36 may push the blades forward. Referring to FIGS. 36-46, generally, while specifically referring to FIGS. 38-39, the piercing assembly 30 may be configured to protect against inadvertent exposure to sharp instruments. A spacer 38 may provide room for operation of a blade assembly 39 or mount 39 holding blades 40 secured thereto.

For example, a “T”-shaped mounting assembly may secure two blades 40 a, 40 b that will eventually slide parallel to the base of the T, and along the same direction of the guide 35 or guide rod 36. In the illustrated embodiment, an aperture in the foot of the T-shaped mount may run along the guide rod 36, driven by the compression spring acting along the length of the rod 36.

The blade assembly or mount 39, together with its attached blades 40 may operate by sliding along an upper surface of the baseplate 42. Two apertures on opposing sides or near opposing edges of the baseplate 42 may receive fasteners to penetrate a pair of corresponding spacers 38. The spacers 38 form a clearance above the baseplate 42 for operation of the mount 39.

A cover 44 or cover plate 44 may include a pair of apertures at or near opposing edges thereof to receive the same fasteners that penetrate the baseplate 42. Accordingly, the cover plate 44, or simply cover 44, is spaced away from the baseplate 42 sufficient distance to receive the mount 39 and attached blades 40 therewithin. Thus, the blade assembly 39 or mount 39 with its attached blades 40 is effectively “garaged” between the baseplate 42, and the cover plate 44. Meanwhile, a compression spring 46 pushes against the base of the T-shaped mount 39, driving the aperture therein along the guide rod 36 captured in the aperture.

A reactor 20 may include a principal containment vessel 50. In one embodiment, a conventional “tin,” or metal can, may be formed by conventional technology available for canning. In other embodiments, the reactor 20 may rely on other structures such as fiber-reinforced composites, cylinders, sealed and flexible but inextensible lattice work, fabrics, or the like, in order to contain the chemical constituents reacting to form nitric oxide.

In one embodiment, tablets, granules, or other configurations of reactants may be placed in a can, sealed to form the reactor vessel 50. An aperture 40 in the vessel 50 may receive a tube 52 acting as a reactor outlet 19. The outlet 19 may conduct nitric oxide generated within the containment vessel 50 to a location outside the insulated container 26 in order to deliver to a line 15.

Various mechanisms may be available for maintaining the integrity of the apparatus 10. In one embodiment, a heat shrinkable wrapping material may be formed in a seamless sleeve. The sleeve may be placed around the apparatus 10, and judiciously penetrated to accommodate the fitting 14, the vent 18, the trigger 16, and so forth. Thereupon, the sleeve 54 may be heated in order to shrink it snugly about the insulated container 26. Thereafter, any breach of the sleeve 54 indicates a lack of integrity of the apparatus 10.

One embodiment of an apparatus 10 in accordance with the invention was formed using expanded polystyrene for the insulated container 26. A fitting 14 to receive a line 15 delivering nitric oxide to a cannula 56 received nitric oxide from a reactor 20 within the insulated container 26. A vent 18 penetrated the cover 32 of the insulated container 26 to vent steam. A trigger mechanism 16 penetrated the cover 32 in order to reach the piercing assembly 30 described hereinabove.

Containers 28 filled with salt water were provided and placed above the piercing assembly 30 and the reactor 20 therebelow. The heaters 22 were placed entirely below the reactor 20, although they may also be wrapped therearound, or even placed on top. However, inasmuch as the heaters 22 tend to vaporize some of the liquid in the containers 28 when released, the heated steam generated below the reactor was effective to heat the reactor 20. Steam rising from heaters thereabove would not ever be in contact with the heaters 22. That is, heat rising with steam originating above the reactor 20, will not contribute as much heat to the reactor 20. The outlet 14 from the reactor was formed of a stainless steel tube 52 penetrating the reactor 20.

In one embodiment, a method of producing nitric oxide may comprise the following steps. A mixture of reactants may be provided consisting essentially of potassium nitrate, sodium nitrite, and chromic oxide. The chromic oxide may be calcined to remove substantially all water bonded thereto. The reactants may be placed in a vessel, or reactor, and any moisture in the vessel may be substantially evacuated. The reactants in the vessel may be heated to a temperature selected to initiate a reaction generating nitric oxide gas. The nitric oxide gas generated may be drawn from the vessel at negative gauge pressure to substantially preclude further heating and limit further reaction of the nitric oxide gas. The nitric oxide gas may be cooled and mixed with a diluent gas to form a mixture breathable by a subject. The breathable mixture may be regulated to substantially ambient temperature and pressure and delivered to the subject to provide a therapeutically safe and effective concentration of nitric oxide gas.

The blades 40 were positioned between the baseplate 42, and the cover plate 44. The guide rod 36 was secured to the baseplate 42 to maintain alignment of the mount 39 as the spring 46 drove the mount 39 forward along the guide rod 36. Upon release of a trigger 16, the mount 39 advanced out from under the cover plate 44, exposing the containers 28 to the sharp blades 40. The blades 40 compromised the containers 28 from below, thus substantially evacuating all the water therefrom. In the experiment illustrated, salt water was used as the liquid within the containers 28. In some experiments, a single container was used. In other embodiments, including experiments conducted, multiple containers 28 filled with liquid were used.

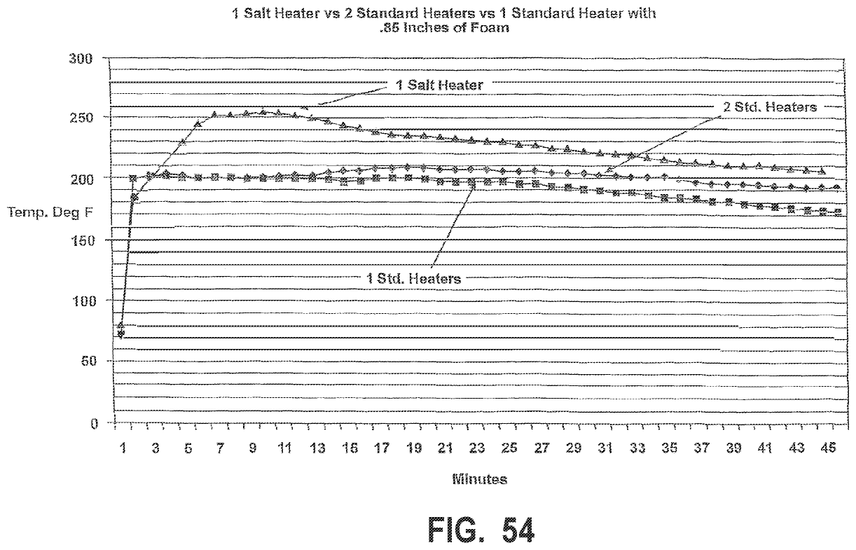

Referring to FIG. 54, in one set of experiments, a single standard heater was used with water, as indicated. In other experiments, multiple heaters 22 were used. In yet other experiments, a single heater was used, but the liquid used to activate the heater 22, was salt water. The chart illustrates the substantial temperature increase due to the use of the ionized salt within the salt water. Throughout the course of the experiment, the temperature was observably higher, and in some instances substantially higher, when salt water was the electrolyte initiating the reaction in the heaters 22. Moreover, a single heater provided more temperature rise in the reactor 20 than twice that amount of chemical (two standard heaters), relying only on water alone as the electrolyte.

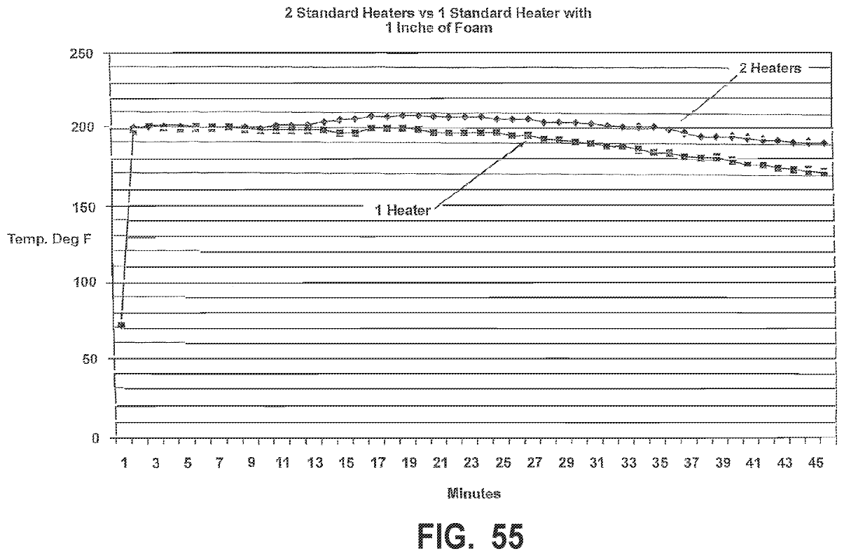

Referring to FIG. 55, one may see that the insulation value of the insulated container 26 has some effect. Nevertheless, in general, a more pronounced effect over the latter part of the subject time results from the addition of a second heater 22.

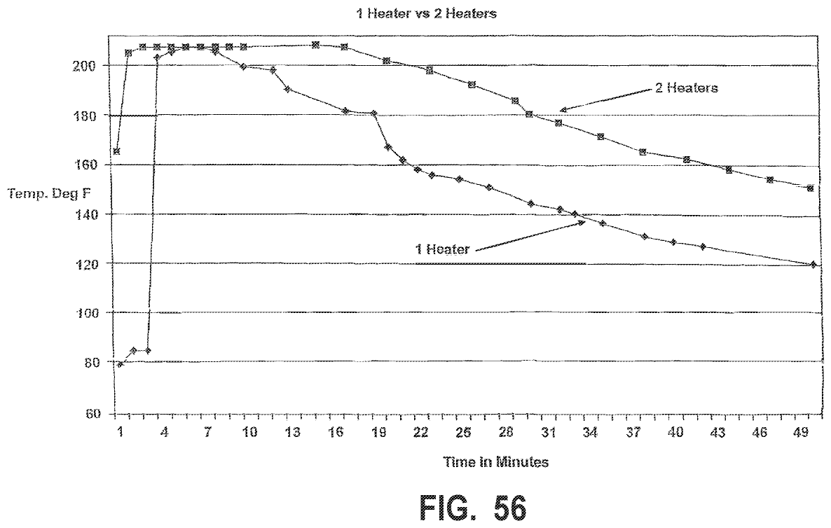

Referring to FIG. 56, in another experiment, the drop off over the subject time period is more pronounced in the last half of the time. Meanwhile, the reactor temperature is maintained close to two hundred degrees Fahrenheit for at least about 20 minutes, when two heaters are used.

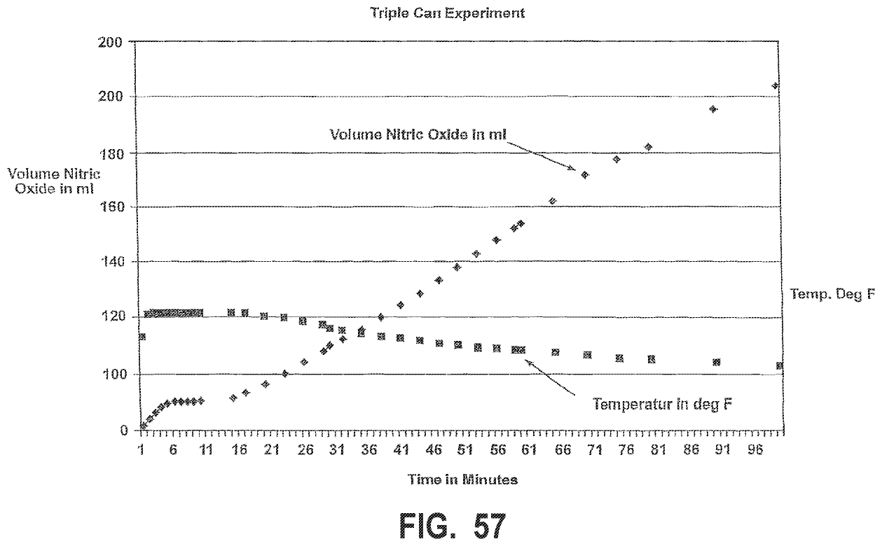

Referring to FIG. 57, the volume of nitric oxide produced, cumulatively, over the operation of an apparatus 10 in accordance with the invention provided the illustrated results. In the chart, temperature was maintained for an extremely long period, considering that a therapy session may typically only require about 30 minutes of nitric oxide generation. The chart illustrates that the volumetric rate of nitric oxide generated was substantially constant, giving rise to a substantially straight slope or line in the time period from about 16 minutes to about 100 minutes. Meanwhile, although the measured temperature dropped during that time period from about two hundred degrees Fahrenheit to just over one hundred degrees Fahrenheit, nitric oxide production did not drop off substantially throughout. Nevertheless, the graph illustrates an apparent decline eventually.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

According to some embodiments, a nitric oxide gel apparatus and method is provided. Gel strips containing reactants capable of reacting to form nitric oxide are maintained separate until application. Upon application, the gel strips are placed in contact with one another, and may mix, or operate by diffusion, to deliver nitric oxide directly to the stream of breathing air of a user. Adhesive strips bonded to a substrate supporting the gel strips may provide for securing the nitric oxide generator directly to an upper lip of a user for breathing the nitric oxide through the nostrils. This invention relates to treatments providing nitric oxide as a vasodilator, and, more particularly, to delivery of gaseous nitric oxide by inhaling.

The discovery of the nitric oxide effect in live tissues garnered a Nobel prize. Much of the work in determining the mechanisms for implementing and the effects of nitric oxide administration are reported in literature including papers, advertising, catalogs, and patents. Much of the work deals with introduction of substances that provide a nitric oxide effect in the body. Still other applications may involve topical preparations introducing nitric oxide. Still other applications rely on bottled nitric oxide gas. Introduction of nitric oxide to the human body has traditionally been expensive.

The therapies, compositions, and preparations are sufficiently expensive to inhibit more widespread use of such therapies. What is needed is a comparatively inexpensive mechanism for introducing nitric oxide in a single dosage over a predetermined period of time. Also, what is needed is a simple introduction method for providing nitric oxide suitable for inhaling.

In accordance with the foregoing, certain embodiments of an apparatus and method in accordance with the invention provide a reactive kit having two compounds, typically disposed in carriers. The two compounds are separated from one another prior to administration. In order to administer the nitric oxide, gel strips are placed in communication with one another beginning a reaction releasing nitric oxide.

An adhesive member may secure the gel strips to a mask or directly to the skin of a user, proximate the nose. A pre-determined rate or amount of nitric oxide may thus be introduced into the breathing air of a subject. Nitric oxide amounts may be engineered to deliver at a comparatively low rate in the hundredths of parts per million, or in a therapeutically effective amount on the order of thousands of parts per million. For example, sufficient nitric oxide may be presented through nasal inhalation to provide approximately five thousand parts per million in breathing air. This may be diluted again (e.g., to about 1200 parts per million) due to additional breathing bypass through nasal inhalation or through oral inhalation.

Some embodiments of an apparatus and method in accordance with the present invention may rely on a layered system having an adhesive strip for securing to an upper lip of a user. A substrate may secure to one side of the adhesive strip while a backing paper, easily removable, may be secured to the opposite side of the adhesive strip. The substrate may support a gel compounded having an appropriate moisture content to support migration of reactants by diffusion therethrough while still maintaining a suitable degree of mechanical integrity. A texture or other holder configuration on a surface of the substrate may support or secure the gel composition.

A second composition in a gel carrier may be sealed or otherwise separated from the first gel composition. For example, the two gel strips may be contained in separate packages. Alternatively, the two gel strips may simply be appropriately separated by an intervening layer, such as a film, paper, or the like. The second layer of gel may be mounted on a substrate as a mechanical integrity precaution, as a mechanism to reduce exposure to ambient air, or both. The first gel strip may be secured by way of the adhesive strip on its substrate to an upper lip of a user. The second gel strip may then be opened and placed in contact with the first gel strip to permit combination of the reactants needed to form nitric oxide. In one embodiment, the reactants may include an acid, such as ascorbic acid, citric acid, or the like. The other reactant may include potassium nitrite.

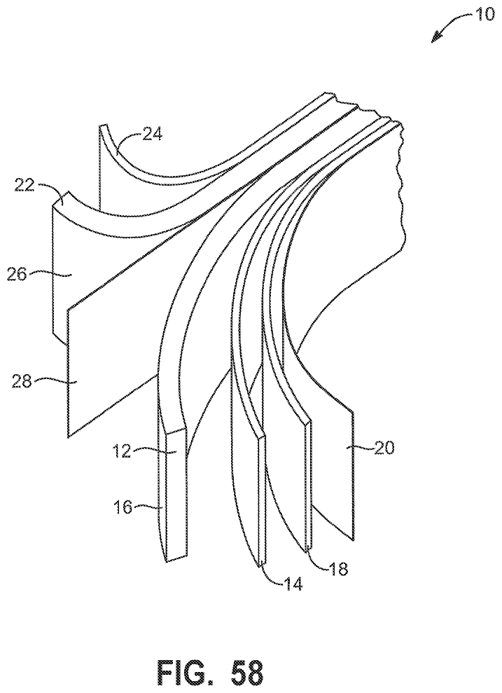

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which FIGS. 58-64 disclose additional advantageous aspects and features.

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention.

Referring to FIG. 58, in one embodiment of an apparatus and method in accordance with the invention, an apparatus 10 may involve a composition formed as a gel layer 12 to have dimensions of thickness, width, and length suitable for creating and sustaining a reaction of an ingredient carried in the carrier gel. For example, a reactive material may be dissolved in a liquid stabilized by gelling agents. Alternatively, reactants may be disposed at a comparatively higher concentration along a surface of a gel carrier.

Meanwhile, the gel carrier may provide sufficient transport for migration of molecules of a reacting composition in order to provide a reactant output. In certain embodiments, the gel may be comparatively thicker in order to provide additional mechanical strength. In other embodiments, the gels may be effectively thixotropic but containing very high levels of hydration, such as fifty to ninety percent water or more. Accordingly, the liquids provide for a concentration gradient to develop, driving each reactant toward the opposing reactant.

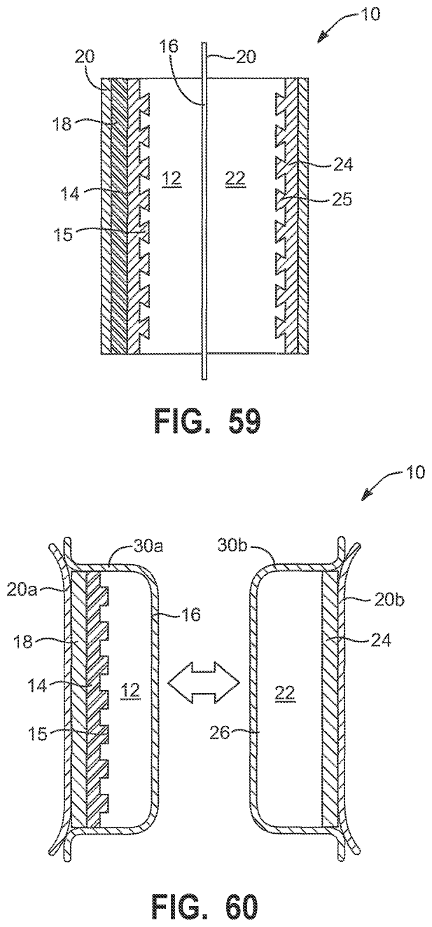

The composition 12 or gel strip 12 may be secured to a substrate 14. The substrate 14 may also include texturing 15 or holders 15 disposed thereon to mechanically stabilize the gel layer 12, as shown more particularly in FIG. 59. The reacting surface 16 or simply the surface 16 of the gel strip 12 may be sealed to prevent exposure to oxygen prior to implementation of a method in accordance with the invention.

On a surface of the substrate 14 opposite the position of the gel strip 12, an adhesive 18 or a layer 18 of adhesive may be applied. The thickness of the layer 18 may be selected to provide securement to an interior surface of a mask, an upper lip of a user, or the like. Various adhesives may be selected to provide adequate securement while also providing suitable release force requirements. Prior to deployment, the adhesive 18 may be covered with a cover 20 or backing 20. For example, paper treated with a polymer to reduce adhesion to the adhesive layer 18 may form the backing 20.

Similar to the gel strip 12, a second composition 22 or gel strip 22 may be positioned to face the initial gel strip 12. Likewise, this new gel layer 22 may be deposited on a substrate 24, with or without holders 25 or texturing 25, as shown more particularly in FIG. 59, to secure compliance of the gel layer 22 with the substrate 24.

A surface 26 of the gel layer 22 operates as both a contact surface 26, and a reaction surface 26 at which, or across which, the reactant species migrate in order to contact one another and react to provide the nitric oxide output of the apparatus 10.

A divider layer 28 may contact the surface 16 of the first gel layer 12, as well as the surface 26 of the second gel layer 22. The two gel layers 12, 22 need not be packaged in the same assembly prior to being placed in contact with one another to begin the desired reaction. However, in one embodiment, a divider layer 28 may be placed in between. Accordingly, the divider layer 28 may be removed from one of the surfaces 16, 26, and then removed from the other surface 26, 16 in order to be thrown away. Thereafter, the two surfaces 16, 26 may be placed in contact with one another, thus initiating the reaction to produce nitric oxide for inhaling. In a typical embodiment, the adhesive layer 18 may be secured to the skin of a user such as just under the nose. In an alternative embodiment, a mask covering the nose, mouth, or both may receive the adhesive 18 in proximity to the nose in order to provide a preselected dose of nitric oxide for inhaling.

In one embodiment of an apparatus and method in accordance with the invention, a mask may be provided with a one-way valve such that breathing out through the mask will not pass air over the apparatus 10, and will thus not discharge nitric oxide overboard. Upon air intake in inhalation, the one-way valve (e.g., flapper valve, check valve, or the like) will open, drawing air past the apparatus 10, and introducing the desired quantity of nitric oxide in the stream of breathing air.

It has been determined that a gram of the first gel 12 placed in contact with a gram of a second gel 22, each containing a suitable quantity of an acid such as ascorbic acid or citric acid, while the opposite layer contains a corresponding amount of potassium nitrite, will provide a quantity of more than five thousand parts per million of nitric oxide in breathing air for over half an hour. With bypass air, this typically dilutes to about twenty-five percent of the original inhaled concentration. Thus, over twelve hundred parts per million in the air of the lungs may be maintained for a time of about thirty minutes.

Referring to FIG. 60, an apparatus 10 may be configured in multiple pieces. For example, in the illustrated embodiment, the apparatus 10 may contain one gel layer 12 on its substrate 14, with or without holders 15 or texturing 15 on the substrate 14 to hold the gel layer 12. In such an embodiment, the adhesive 18 is mounted to the substrate 14 opposite the gel layer 12. Meanwhile, a cover 20 a or backing 20 a may cover the adhesive 18 in order to maintain cleanliness and the like.

Meanwhile, another cover 30 a may be applied to seal the exposed outer surface 16 of the gel layer 12, as well, against the atmosphere and environment. In certain embodiments, the cover 30 a may be sealed to the backing 20 a in order to provide a completely sealed package. The substrate 14 may be configured to provide additional support or sealing along the bottom and end thereof. For example, in the illustrated embodiment, the cross-sectional view illustrates a bottom portion of the substrate 14 that may serve to support and seal that portion of the substrate 14 and gel strip 12 against oxygen, even in use.

Meanwhile, the substrate 24 corresponding to the gel layer 22 may have a similar configuration to matingly engage the substrate 14 and to place the gel layer 22 in contact with the gel layer 12.

For example, the surface 16 may be placed in contact with the surface 26 of the gel layer 22. Meanwhile, the backing 20 b need only serve as a mechanism to seal the cover 30 b around the gel layer 22.

The layups illustrated in FIGS. 58-60 may typically begin with a selection of reactants, followed by providing a suitable substrate. Reactants may include, for example, an acid of modest strength such as citric acid or ascorbic acid. Meanwhile, such an acid may react appropriately with potassium nitrite. Other nitrogen compounds may also serve. In certain embodiments, the reactants may be provided as solids. In other embodiments, the reactants may be provided as solutions in a liquid, such as water. In yet other embodiments, the reactants may be provided in a solution mechanically stabilized as a gel, such as a water-based gel.

Providing a substrate may include selecting a material to operate with the gel layers 12, 22. The reactants may be mixed with the gel in order to provide a solution. Alternatively, the reactants may be mixed dry, and a liquid or gel may be introduced in order to carry chemical species and ions during reaction.

The layups may be created by providing the gel composition, for each of the gel layers 12, 22, and disposing them along the substrates 14, 24, respectively. Adhesive may be added behind the substrate 14 at any appropriate time, and the sealing covers 20, 30 may be added thereafter. The development of the apparatus 10 may begin with removal of both seals or covers 20, 30, by separation from one another.

By positioning the surfaces 16, 26 against one another, the reactants may begin to react. In certain embodiments, the shape of the surfaces 16, 26 may be designed to promote a certain degree of mixing therebetween upon contact. For example, the surfaces 16, 26 may actually be formed to be smooth, splined, undulating, saw-toothed, or the like. Accordingly, introducing the two gel layers 12, 22 to one another may actually involve mixing them with one another.

After the gel strips 12, 22 are unsealed and placed in contact, they may be deemed activated. The adhesive 18 may then be applied to the skin of a user proximate the nose, or mouth. Likewise, the adhesive may be used to retain the apparatus 10 against the interior of a breathing mask.

Breathing masks are available in the art and are used for oxygen provision, continuous positive airway pressure apparatus, and the like.

The reactants in the gel strips 12, 22 have been found to provide adequate levels of nitric oxide production. For example, a gram of gel 12 combined with a gram of gel 22 have been found to provide a five thousand parts per million dose for over thirty minutes to a user.

As the reactants are eventually consumed, the rate of production of nitric oxide may decay to a useless level. Below some threshold value, the apparatus 10 may be deemed inappropriate or expended. Accordingly, the adhesive 18 may be removed from its location during deployment and the apparatus 10 may be disposed of appropriately.

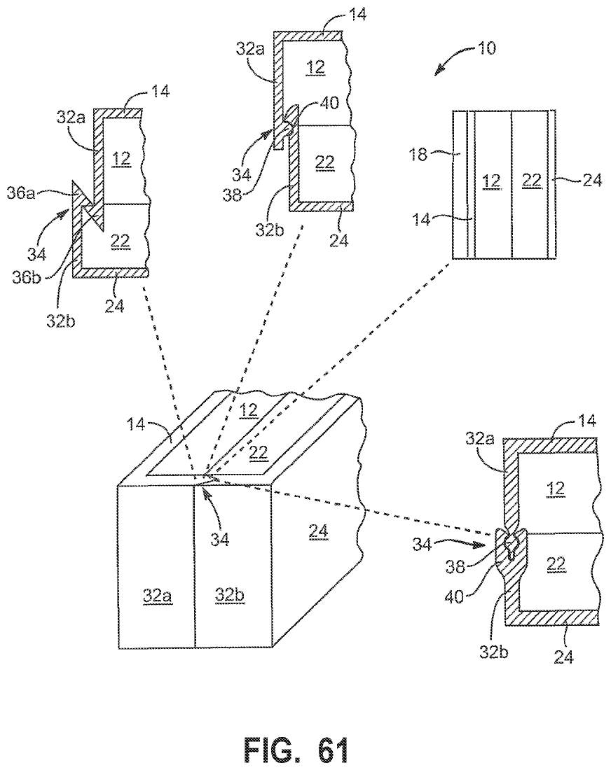

Referring to FIG. 61, in certain embodiments of an apparatus 10 in accordance with the invention, the substrates 14, 24 may mechanically engage one another to provide selected sealing and opening. For example, in the contemplated deployment directly near a breathing opening (e.g., nostrils, mouth), it may be desirable to permit the nitric oxide production to exit only in a single direction.

This also has the effect of sealing other surfaces against additional reaction with available environmental oxygen. For example, if oxygen is allowed to come in contact with the nitric oxide, the nitric oxide may become nitrogen dioxide, or some other compound of nitrogen. Although these compounds may not be harmful, any overreaction creating a compound of nitrogen having more than a single oxygen for each nitrogen atom is a waste of reactant material.

Accordingly, the substrate 14 may be formed as a latching structure, having ends 32, having an engagement mechanism 34 to seal them together. For example, a barb or ratchet-like connection may provide that once the two substrates 14, 24 have engaged to within a certain proximity, they will be latched together by the latching device 34. In other embodiments, a detent such as a bump, corrugation, boss, or the like may be designed to engage a recess in a corresponding substrate 24.

For example, in the left central illustration of the alternative embodiments of FIG. 61, a detent is engaged by a recess. In the leftmost embodiment, a ratchet or barb engages the two substrates 14, 24 with one another. In the right central embodiment, the adhesive properties of the gel layers 12, 22 themselves nearly maintain themselves in contact.

In such an embodiment, the outer edges of the gel layers 12, 22 may be treated with an oil, a film, a non-reactant substance, another polymer, or the like. Nevertheless, the gel layers 12, 22 are not sealed so firmly as in the other embodiments. Likewise, in the embodiment of FIG. 61, the rightmost illustration shows a detent in which a receiver receives a detent, capturing the detent from both sides thereof. Typically, a barb 36 a may be used opposite another barb 36 b relying only on the resilience of the material of the substrates 14, 24, respectively. Typically, a detent 38 may operate with respect to a relief 40 in a corresponding piece by the same manner. However, in certain embodiments, a more affirmative (e.g., forceful) bonding may occur if the detent 38 is fully captured by the relief 40 completely surrounding the detent 38 as illustrated in the rightmost illustration.

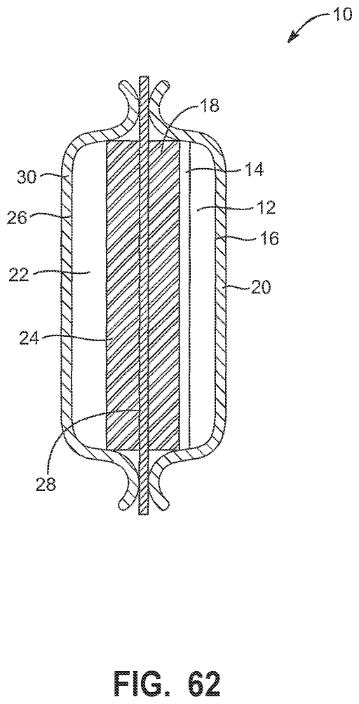

Referring to FIG. 62, in certain embodiments, a single package may implement the apparatus 10 in one embodiment. In the illustration of FIG. 62, a gel layer 12 is disposed on a substrate 14 provided with an adhesive 18. The surface 16 is sealed by the cover 20. Meanwhile, a gel layer 22 is disposed along the substrate 24, having its reaction surface 26 sealed by a cover 30.

Meanwhile, a divider 28, operates in a manner almost opposite that of the divider 28 of FIGS. 58-59. In FIGS. 58-59 the divider serves to separate the gel layers 12, 22. In the illustrated embodiment FIG. 62, the divider 28 serves to divide two packages, each separately sealed between its respective cover 20, 30, and the divider 28. Thus, the adhesive 18 may be peeled from the divider 28, as described hereinabove.

Meanwhile, the substrate 24 may be removed from the divider 28, and the surfaces 16, 26 may be juxtaposed and placed in contact. In certain embodiments, the substrate 24 may act as the divider 28. However, one benefit of having the divider 28 as a separate element is that it may extend beyond the operational dimensions of the gel layers 12, 22, and their respective substrates 14, 24, in order to effect the seals with the respective covers 20, 30.

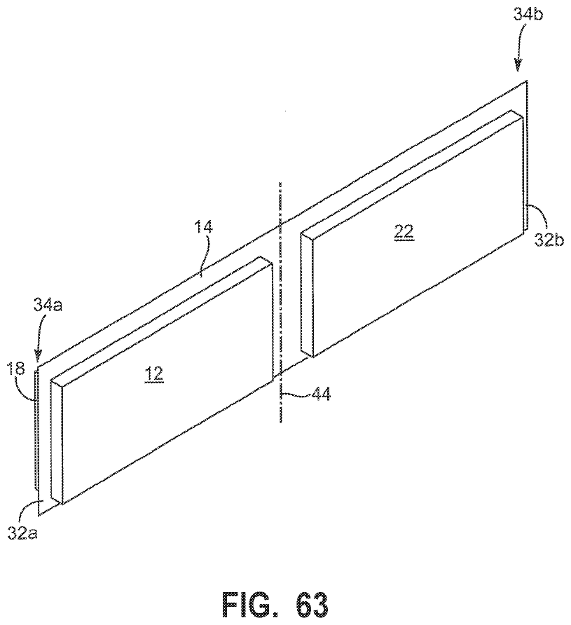

Referring to FIG. 63, in one embodiment of an apparatus and method in accordance with the invention, an apparatus 10 may be formed on a single substrate 14. In this embodiment, the packaging may be that reflected in FIG. 60, FIG. 62, or an alternative packaging. In the illustrated embodiment, the gel layers 12, 22 may be maintained separately from one another in order to prevent any premature reaction. Meanwhile, upon removing any covers 20, 30 from the gel layers 12, 22, the substrate 14 may be folded about a fold line 44 near the center thereof, placing the gel strips 12, 22 in contact with one another along their reaction surfaces 16, 26, respectively.

In the illustrated embodiment, the ends 32 a, 32 b may be provided with a latch device 34, such as a barb 36, detent 38 and relief 40, or the like, as illustrated hereinabove. The adhesive layer 18, may be applied to the substrate 14 opposite the gel layer 12. The use of a latching device 34 along with the substrate being bent about the fold line 44 may maintain the gel strips 12, 22, in close proximity for reaction purposes.

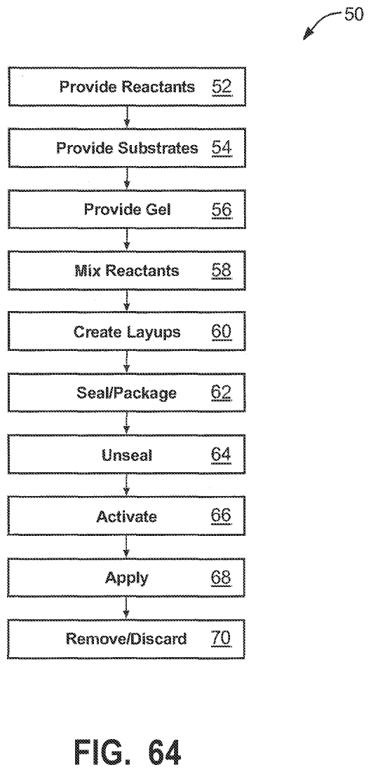

Referring to FIG. 64, one embodiment of an apparatus and method in accordance with the invention may rely on a process 50, or some part thereof. For example, providing 52 reactants may be done by providing solids, granules, powders, or the like sufficiently dry that there is very little reactivity therein. Likewise, providing 52 reactants in a liquid or gel provides an enabler, a transport medium or carrier to carry either a particulate or a dissolved reactant. Reactants typically provided may include a carrier such as water, another liquid, a gel, or the like, along with an acid, such as citric acid or ascorbic acid, and a compound of nitrogen such potassium nitrite.

Providing 54 a substrate may involve providing a material to support the reactants. Typical substrates may include fabric soaked in a reactant, a batting, such as cotton or a synthetic bat, a strip, a box, or the like. Providing 56 a gel may involve gelling a solution already containing a reactant, or providing a carrier material for receiving a reactant. Other embodiments may use other mechanisms to introduce, and separate or mix an active ingredient from a carrier gel. By gel is meant simply a stabilized liquid that is mechanically capable of supporting its weight. Gels may range from thixotropic fluids to rheological solids with viscoelastic properties.

Mixing 58 the reactants may involve mixing the reactants with one another, mixing the reactants with a gel, or otherwise providing them in a disposition suitable for ready application. Creating 60 the layups may involve the processes described hereinabove for providing the gel layers 12, 22 on the substrates 14, 24, respectively, along with their respective covers 20, 30, and the like.

Sealing 62 may involve using the covers, other materials, coatings, films, and the like, including foils, plastics, oils, and the like to seal the reactants against the environment and against one another.

During deployment, a user will typically unseal 64 a package containing the apparatus 10. Activation 66 typically involves placing the reactants in contact with one another. This may be done chemically or mechanically. For example, the gel layers 12, 22 may be disposed with respect to one another as described hereinabove. The gel layers may be placed in contact. They may be forced into one another. They may be shaped in such a way that there tends to be mixing. They may be placed in proximity to one another and then mixed somewhat with one another, or the like.

Accordingly, the active ingredients may be activated 66 to begin their reaction with one another, producing nitric oxide in the process. Applying 68 the apparatus 10 to a user for purposes of therapy may involve simply securing the adhesive 18 to the body of a user near the nostrils in order to promote breathing of the nitric oxide as it is generated.

After a preselected period of time for dosing, or upon expiration of the active ingredients, the apparatus 10 may be removed 70 from a user. Inasmuch as nitric oxide and nitrogen dioxide have the tendency to color the gels 12, 22, the presence of a dark rust or dark brown color indicates that the reactants are used up. Is has been observed that the gel will initially create a white froth as gases are generated within the gel. Eventually, a pink color overtakes the white reflection of a refraction of light from the air bubbles. The pink color eventually gives way to brown, which eventually becomes dark brown.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

According to some embodiments, a nitric oxide reactor and distributor apparatus and method is provided. A reaction and distribution system may include a distributor securable near or in a path correspond to a breathing passage such as the nostrils or the mouth of a user for delivering nitric oxide therapy thereto. The distributor may contain an internal reactor for creating the nitric oxide from reactants. Alternative embodiments may rely on a line delivering nitric oxide to the distributor from a remote generator such as a cannister carried in a pocket or placed/at the bedside of a user. This invention relates to treatments providing nitric oxide as a vasodilator, and, more particularly, to generation and delivery of gaseous nitric oxide for inhaling.

The discovery of the nitric oxide effect in live tissues garnered a Nobel prize. Much of the work in determining the mechanisms for implementing and the effects of nitric oxide administration are reported in literature including papers, advertising, catalogs, and patents. Much of the work deals with introduction of substances that provide a nitric oxide effect in the body. Still other applications may involve topical preparations introducing nitric oxide. Still other applications rely on bottled nitric oxide gas. Introduction of nitric oxide to the human body has traditionally been expensive.

The therapies, compositions, and preparations are sufficiently expensive to inhibit more widespread use of such therapies. What is needed is a comparatively inexpensive mechanism for introducing nitric oxide in a single dosage over a predetermined period of time. Also, what is needed is a simple introduction method for providing nitric oxide suitable for inhaling.

In accordance with the foregoing, certain embodiments of an apparatus and method in accordance with the invention provide a reactive kit having two compounds, typically disposed in carriers. The two compounds are separated from one another prior to administration. In order to administer the nitric oxide, reactants are mixed in with one another beginning a reaction releasing nitric oxide.

An adhesive member may secure a distributor to a mask or directly to the skin of a user proximate the nose. Nitric oxide may thus be introduced into the breathing air of a subject. Nitric oxide amounts may be engineered to deliver at a comparatively low rate in the hundreds of parts per million, or in a therapeutically effective amount on the order of thousands of parts per million. For example, sufficient nitric oxide may be presented through nasal inhalation to provide approximately five thousand parts per million in breathing air. This may be diluted due to additional bypass breathing through nasal inhalation or through oral inhalation.

One embodiment of an apparatus and method in accordance with the present invention may rely on a small reactor feeding a distributor secured to an upper lip of a user. A diffuser may secure to one side of an adhesive strip, while a treated backing paper, easily removable, may be secured to the opposite side of the adhesive strip. A reactor may be sized to contain reactants as solids, liquids, or gels compounded to have an appropriate moisture content to support reaction of reactants. A second reactant composition in a carrier may be sealed or otherwise separated from the first reactant composition. For example, the two reactants may be contained in separate volumes. Alternatively, reactive solids may simply be appropriately combined dry, or even separated by an intervening layer, such as a film, paper, or the like. The reaction may begin upon introduction of a liquid transport material to support ionic or other chemical reactions. The reactants held in separate, sealed volumes may be opened and mixed or otherwise placed in contact with one another to permit combination of the ingredients needed to form nitric oxide. In one embodiment, the reactants may include an acid, such as ascorbic acid, citric acid, or the like as a hydrogen donor. The other reactant may include potassium nitrite, sodium nitrite or the like.

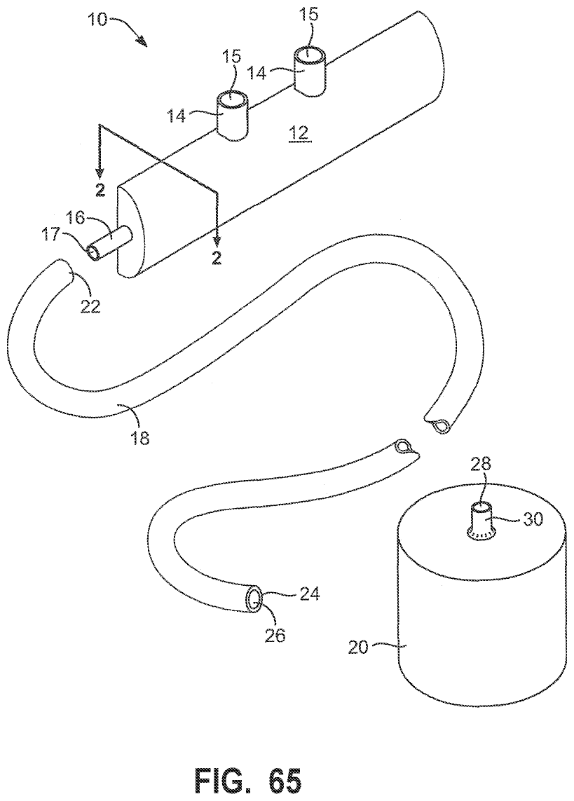

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which FIGS. 65-69 disclose additional advantageous aspects and features.

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention.

Referring to FIG. 65, an apparatus 10 in accordance with the invention may include a vessel 12 or distributor 12. The distributor 12 may be configured to be flexible or may be pre-formed to fit the anatomy of a user. Typically, the distributor 12 will be placed on the upper lip of a user to provide the outputs 14 (e.g., output ports 14, or simply ports 14) access to the nostrils of a user during breathing. Each of the outputs 14 has an opening 15 for delivering nitric oxide directly into the nostrils of a user. Typically, sufficient clearance provides a bypass for air in addition to the nitric oxide from the distributor 12.

In certain embodiments of an apparatus in accordance with the invention, a distributor 12 may include a port 16 to operate as an input 16 for receiving nitric oxide from another source. For example, the port 16 may have an opening 17 for receiving from a line 18 a supply of nitric oxide.

In the illustrated embodiment, a reactor 20 provides a supply of nitric oxide to the distributor 12. As illustrated, one end 22 of a line 18 may connect to the input port 16 of the distributor 12. The opposite end 24 of the line 18 connects to the reactor 20. The opening 26 of the line 18 provides a lumina 26 value or passage 26 for passing the nitric oxide gas from the opening 28 of the fitting 30 on the reservoir 20.

In certain embodiments, the reactor 20 may be manufactured in a single-dose size. Accordingly, the distributor may be reused or disposed of. The reactor 20 may typically be disposed of after a single use. Circumferential hoop stresses are not high. Accordingly, the distributor 12, the line 18, and the reactor 20 may all be fabricated from comparatively lightweight and inexpensive materials such as plastic. Parts may be cast, molded, vacuum formed, assembled from film, or the like.

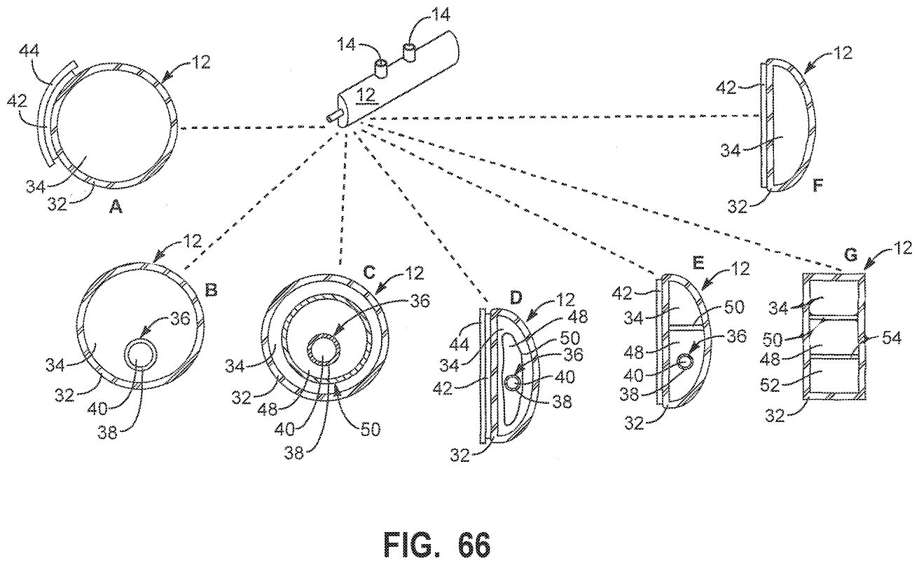

Referring to FIG. 66, the distributor 12 may be configured in various cross-sectional shapes. For example, the distributor 12 may typically have a principal wall 32 enclosing a chamber 34 or volume 34 containing the necessary materials for therapy. In certain embodiments, the chamber 34 may simply act as a manifold or distributor channel conducting nitric oxide gas. In other embodiments, the chamber 34 may completely enclose the reaction constituents and structures. Thus, the distributor 12 may serve as both a distributor 12 and reactor 20 in a single, integrated apparatus 10.

In various embodiments, the chamber 34 may include a vessel 36 inside or completely enclosed within the wall 32 and chamber 34 of the distributor 12. The internal vessel 36 may have a wall 38 that is permeable or impermeable. In certain embodiments, the vessel 36 may have a wall 38 formed of glass to maintain the vessel 36 sealed from the contents of the chamber 34. Accordingly, upon fracture of the wall 38, the contents of the vessel 36 may be spilled into the chamber 34 to mix with other reactants.

In certain embodiments, the chamber 40 formed by the wall 38 of the vessel 36 may contain a reactant. In other embodiments, the chamber 40 may simply contain a liquid. In yet other embodiments, the chamber 40 may contain dry ingredients that will become exposed to liquid from the chamber 34 upon fracture of the wall 38 and exposure of the chamber 40 to the contents of the chamber 34. All the foregoing roles can likewise be traded or reversed.

As can be seen, reactants may be separated to render them inactive. The reactants may later be combined to render them active and initiate a reaction. Likewise, the reactants may be maintained in proximity to one another in the chamber 34, the chamber 30, or both, or one may be maintained in a chamber 30, 34 dry and another wet. However, once both reactants are present in the presence of a liquid (e.g., transport fluid) in the opposite chamber 34, 30, the reaction to release nitric oxide may begin.

Any of the embodiments of FIG. 66 may be provided with an adhesive strip 42. One function of the adhesive strip is to secure the distributor 12 proximate the nostrils of a user in order that the distributor 12 may deliver nitric oxide through the openings 15 of the output ports 14. For clarity, the adhesive strip 42 has not been illustrated in every embodiment, although it may. Nevertheless, each of the embodiments may be provided with an adhesive strip 42. Meanwhile, any of the distributors 12 may be secured by some other method.

For example, the distributor 12 may be positioned within a mask covering the nose, the mouth, or both. Likewise, the distributor may be positioned by an air inlet to such a mask. In other embodiments, the distributor 12 may be positioned directly near the mouth, nostrils, or both. Accordingly, the output ports 14 may be shaped to accommodate the positioning thereof for delivery of nitric oxide to the breathing air stream of a subject.

In certain embodiments, an additional volume 48 may be separated within the chamber 34. For example, a layer 50 or wall 50 may seal the reactants away from one another. The wall 50 may be formed of a film, such as a molecular sieve. Such molecular sieves are available from suppliers and may be formed of various materials. One film produced under the trademark Nafion™ operates as a molecular sieve.

The value of a molecular sieve is that it is configured to have a pore size that will not permit passage of a compound of nitrogen having more than a single oxygen. Accordingly, only nitric oxide may pass through the molecular sieve. The molecular sieve, thus restrains the reactant liquids, any particulate matter, and all constituents larger than the nitric oxide molecule. Thus, the nitric oxide molecule may pass through the wall 50 and exit the chamber 34 through the output ports 14.

In yet other embodiments, the basic chamber 34 may be separated away from an additional chamber 48 or volume 48 by a seal 50 or wall 50. Meanwhile, the main chamber 34 may be further subdivided to create an additional volume 52 separated by a wall 54 or seal 54. In the illustrated embodiment, a volume of a first reactant in the chamber 48 is separated entirely from a volume of a second reactant in a chamber 52. Meanwhile, the remaining volume of the chamber 34 may be left as air space to receive the reactant gas passing through the molecular sieve of the layer 50.

Referring to FIG. 66, embodiment A is configured simply as a distributor 12 in which the chamber 34 enclosed by the wall 32 merely passes the nitric oxide for distribution to the output ports 14. Meanwhile, an adhesive layer 42 is bonded to the wall 32 and may be secured to the skin of a user upon removal of a layer 44 or cover 44 protecting the adhesive properties of the layer 42 from their environment during handling.

Embodiment B of FIG. 66 includes an additional chamber 40 separated by a wall 36. In this embodiments, one reactant may occupy the principal chamber 34, while a second reactant occupies the chamber 40 within the wall 36. If the wall 36 is formed of glass, then bending the distributor 12 may fracture the wall 36, exposing the reactants in the chamber 34 to the reactants in the chamber 40. Accordingly, the relative sizes of the chambers 34, 40 may be configured according to the necessary and appropriate quantities of the reactants contained therein, respectively.

The reactants in the chambers 34, 40 may be dry, wet, or one may be dry and one may be wet. Likewise, one chamber 34, 40 may contain both reactive ingredients mixed together but completely dry, while the other chamber 40, 34 contains a liquid capable of acting as a transport medium and thus activating the reaction between the dry ingredients.

Substantially all the illustrated embodiments for a reactor 20 or for a distributor 12 may benefit, as appropriate, from one of the foregoing configurations of dry, wet, or wet and dry ingredients, or dry ingredients and a wet transport material 12.

Embodiment C provides for a distributor 12 having one volume 48 enclosed by a molecular sieve layer 50. Meanwhile, a wall 36 encloses another chamber 40 containing another reactant. In this embodiment, the remainder of the volume of the chamber 34 outside the wall 50 of the molecular sieve is available as free space. Meanwhile, all reactants are contained within the molecular sieve layer 50.

A fracture of the wall 36 may release the reactants from the chambers 40, 48 to mix with one another and react. Meanwhile, the molecular sieve layer 50 contains all the reactants, as well as species of reaction that may be other than nitric oxide. Typically, nitric oxide is the principal output of the proposed reactants. Nevertheless, when exposed to the reaction process too long or when provided with outside oxygen, nitric oxide may become a more oxygenated reactant of nitrogen.

Embodiment D illustrates a more easily bendable shape, that may be more comfortable and more practical for forming about the upper lip of a user. For example, in any illustrated embodiment, any of the materials used to form the wall 32 of the chamber 34 may be comparatively rigid, moderately flexible such as a soft plastic or elastomer, or very flexible such as the materials used to form a toothpaste tube or other collapsible tube for containing a paste or liquid. Accordingly, the distributor 12 may be formed to fit the lip a user. Internal materials such as a wire imbedded in part of the wall 32 may facilitate bending the distributor 12 to a specific and permanent shape. Meanwhile, the adhesive strip 42 may secure a comparatively weak and soft material to the lip of a user and thus maintain the desired shape.

In embodiment D, the molecular sieve layer 50 may be a flexible film that provides additional space in the chamber 34 as gas accumulation space, while still containing the volume 48 of one reactant. In the illustrated embodiment, the chamber 40 is maintained within the wall 38 of a vessel 36. If the vessel 36 has a rigid wall 38, such as one formed of glass, a simple bending of the distributor 12 may permit mixing of the reactants in the chambers 40, 48 and discharge of the nitric oxide reactant through the wall 50 to accumulate in the remaining dry portion of the chamber 34 for ultimate discharge through the output ports 14.

Embodiment E provides a molecular sieve layer 50 permanently disposed across the chamber 34 separating a portion of the chamber 34 from a cavity 48 or volume 48 containing a reactant. Thus, a portion of the chamber 34 remains dry, while a portion is separated off as the volume 48 for containing a reactant. In this embodiment, the volume 40 is likewise contained by a wall 38 as a separate vessel 36 containing one of the reactants. Typical reactants are moderate acids such as citric acid, ascorbic acid, acetic acid, or the like. Meanwhile, typical reactants may involve compositions of nitrogen such as potassium nitrite, sodium nitrite, or the like. Reactants may be disposed as granules, powders, liquids in solution, solutions gelled to thixotropic consistency, or the like.

Embodiment F illustrates a distributor 12 that contains no reactants and does not act as a reactor 20 or reactant chamber 34. Rather, the chamber 34 of embodiment F is simply an empty cavity for distributing nitric oxide to the output ports 14.

Embodiment G may actually be configured in various shapes. However, as a manufacturing matter, alignment, assembly, and the like may be best served by more linear envelopes rather than curved ones. Nevertheless, the arrangement of embodiment G may actually be imposed on other shapes. In this embodiment, the chamber 34 may be separated by a molecular sieve layer 50 from a chamber 48 containing one reactant. Meanwhile, another seal 54 or wall 54 may separate the ingredients in the chamber 48 from the volume 52 or chamber 52 containing the second ingredient.

The entire reaction is contained within the wall 32, but the individual wall 50 acts a molecular sieve and will not be ruptured. By contrast, in order to initiate the reaction, the wall 54 may be compromised by perforating, fracture, rupture, tearing, cutting, or the like. Meanwhile, the remainder of the chamber 34 provides head space for the gas to accumulate for discharge through the output ports 14.

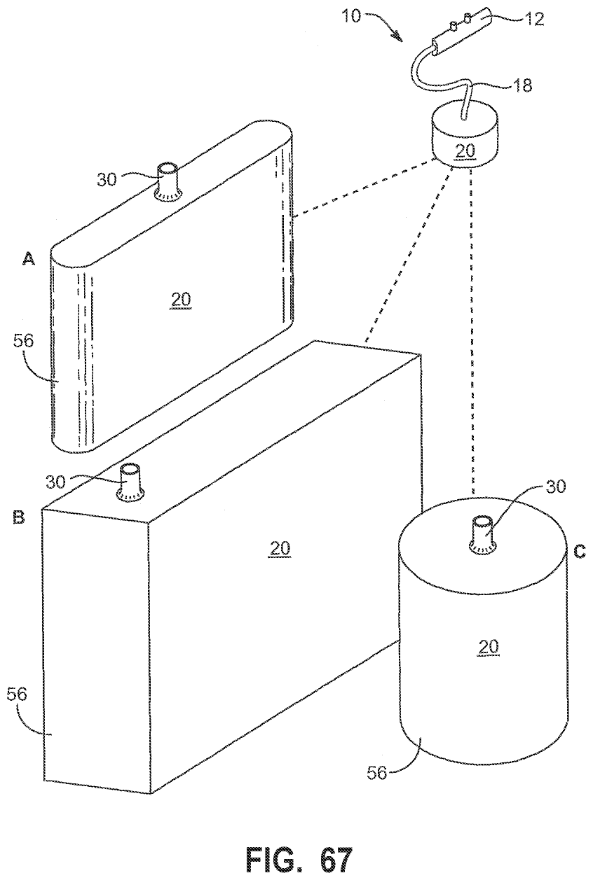

Referring to FIG. 67, a reactor 20 in the apparatus 10 may be configured in any suitable shape. Circular cross-sections tend to provide an equalization of hoop stresses. However, the reaction of materials contemplated for an apparatus 10 in accordance with the invention need not operate at an elevated pressure. Typically, the reaction may occur at about ambient conditions.

In embodiment A of FIG. 67, the reactor 20 may be configured as a rounded, yet somewhat flattened device having an aspect ration of width to thickness that is substantially larger than unity. Thus the width is more than the thickness, and in the illustrated embodiment is several times the thickness. Meanwhile, the aspect ratio of height to width may be selected according to space available in a convenient location for holding the reactor 20. For example, embodiment D may be a suitable configuration for setting on a table top. By contrast, embodiment A may be better suited for slipping into a shirt pocket, jacket pocket, or the like for portability. Meanwhile, the reactor 20 of embodiment C may be suitable for holding in a jacket pocket, or sitting on a night stand beside a bed or other flat surface.

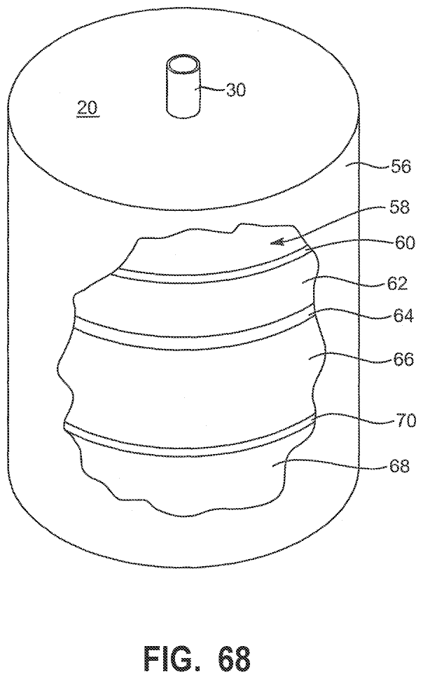

Referring to FIG. 68, any of the reactors 20 of FIG. 67 may be configured to contain any or all of the chambers of FIG. 66. The reactor 20 may enclose various individual volumes. For example, in the illustrated embodiment, a volume 58 is enclosed within the wall 56 of the reactor 20. The volume 58 is bounded below by a layer 60 or sieve layer 60.

Optionally, a region of expansion space 62 may exist above a closure layer 64. The layer 64 initially forms a retainer or seal 64 to contain the volume 66 of a first reactant. The first reactant volume 66 is separated from a volume 68 containing the second reactant by a seal 70 that may be ruptured or otherwise compromised to initiate a reaction.

The closure layer 64 may be permeable. Alternatively it may be sealed impervious, to be breached in preparation for initiating the reaction in the reactor 20. It may be burst or otherwise opened or by the reaction.

In one embodiment, the layers 64, 70 may be formed of a polymer film, wax, or the like capable of maintaining the volumes 66, 68 separated from one another with their reactants. A mechanism such as a plunger, perforator, mixer, spatula, or other apparatus extending through the wall 56 may serve to break, rupture, tear, cut, or otherwise compromise the layer 70. Likewise, the layer 64 may be so opened and compromised in order to make the expansion space 62 available to the reactants.

The reactants in the volumes 66, 68 may be solid, liquid, one of each, or some other combination. For example, an additional layer, possibly even including the volume 62, may contain a liquid to provide a transport fluid for dry reactants in the volume surface 66, 68.

By whatever mechanism, the layers 64, 70 may be opened to expose the volumes 66, 68 with their reactant contents to one another in order to activate the reactor 20 and begin the chemical reaction to produce nitric oxide. Nitric oxide passes through the molecular sieve layer 60, which may be optional, but is useful in maintaining the purity of nitric oxide. The molecular sieve 60 or the layer 60 may include not only a molecular sieve, such as a film or solid layer, but may also include any other barrier materials suitable to maintain reactants outside of the collection volume 58 collecting the nitric oxide.

Ultimately, the nitric oxide in the volume 58 is passed through the fitting 30 into a line 18 for delivery into a distributor 12. Notwithstanding the illustrated embodiment of FIG. 68, any suitable shape may be used for the cross-section of the reactor 20. Accordingly, the reactor of FIG. 68 may actually be configured according to the relations, shapes, or both illustrated in any of the alternative embodiments illustrated in FIGS. 65-67.

In one alternative embodiment, the wall 56 may be highly flexible. Moreover, shape may be selected having an aspect ration of length to width that is comparatively larger than unity. The ratio of width to thickness may also be selected to be substantially larger than unity. Accordingly, the reactor 20 may be configured as a comparatively long, narrow tube, of a comparatively smaller thickness. Accordingly, the reactor 20 may be rolled up like a toothpaste tube or kneaded in order to rupture the seal layers 64, 70 and to mix the reactants in the volumes 66, 68.

If the volumes 66, 68 are filled with solutions, for example, reactants disposed in a solute liquid, or freely flowing gel, then mixing may readily occur. In other embodiments, diffusion alone may control the migration of reactant species between the volumes 66, 68. Thus, sealing layers 64, 70 may be formed, dividing the chambers or volumes 66, 68 containing reactants, which may then be extruded, mixed, drawn, flown, stirred, or otherwise introduced to one another to increase the available species participating in the reaction.

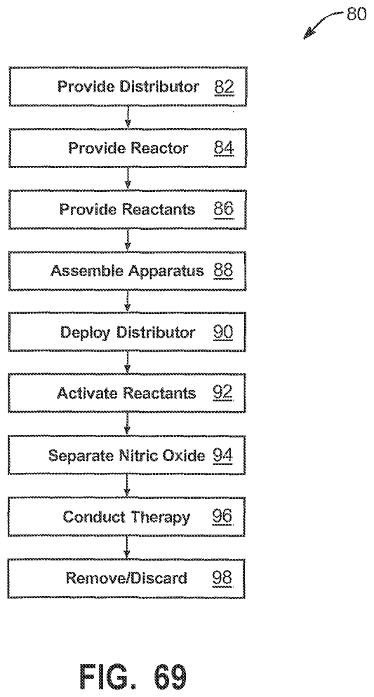

Referring to FIG. 69, one embodiment of an apparatus and method in accordance with the invention may rely on a series of process steps constituting a method 80 or process 80. For example, providing 82 a distributor 12 may involve any one or more of the required tasks of identifying materials, selecting a shape, selecting a cross-sectional profile and area, selecting aspect ratios of length to width to thickness, and determining the structural and mechanical characteristics for such a distributor 12. Accordingly, providing a distributor 12 may involve design, engineering, manufacture and acquisition of such a device.

Providing 80 a reactor may involve selection of materials, selection profile and of cross-sectional area, engineering, design, fabrication, acquisition, purchase, or the like of a reactor 20 in accordance with the discussion hereinabove.

Providing reactants 86 may include selection of reacting species, selecting a configuration, such as granules, powder, liquid, a solution, or the like. Likewise, the particular configuration of a solidous configuration of reactants may involve selecting a sieve size for the particles. This site can affect chemical reaction rates. Thus, selecting or otherwise providing 86 reactants for the reactor 20 may involve consideration of any or all aspects of chemistry, reaction kinetics, engineering, design, fabrication, purchase or other acquisition, delivery, assembly, or the like.

Assembling 88 the apparatus may involve a single distributor as an integrated embodiment as described with respect to FIG. 66, or assembly of a reactor, with a feed line 18, connected to a distributor 12. Likewise, assembling 88 may also include the disposition of reactants within various locations within a reactor 20, distributor 12, or the like as discussed hereinabove.

Deploying 90 the distributor may involve opening up a package provided during assembly 88 of the apparatus 10. For example, assembling 88 may also include packaging. Accordingly, deploying 90 may involve opening packages, unsealing components, and otherwise rendering the apparatus 10 ready for use. Likewise, deploying 90 the distributor 12 may involve positioning the distributor 12 with respect to a user, including, for example, adhering the distributor 12 to the skin of a user proximate the nostrils for inhaling the nitric oxide provided by the distributor 12.

Activating 92 the reactants in the reactor 20 may involve, either adding a liquid, mixing the reactant components together, dispersing individual reactants in respective solutes to provide solutions for mixing, adding a liquid transport carrier to dry ingredients in order to initiate exchange between reactants, a combination thereof, or the like.

Likewise, activation 92 of the reactants may also involve opening valves, opening seals, rupturing or otherwise compromising seals as described hereinabove, or otherwise moving or manipulating reactants with or without carriers in order to place them in chemical contact with one another.

In certain embodiments, nitric oxide may be separated 94 from the reactants themselves. For example, the concept of a molecular sieve 60 was introduced hereinabove as one mechanism to separate 94 nitric oxide form other reactants and from other species of nitrogen compounds. In other embodiments, pumps, vacuum devices, or the like may also tend to separate 94 nitric oxide. Accordingly, in certain embodiments, a suitably sized pump may actually be connected to the reactor 20 in order to draw nitric oxide away from other species of reactants or reacted outputs.

Conducting 96 therapy using nitric oxide may involve a number of steps associated with delivery and monitoring of nitric oxide through the distributor 12. For example, in certain embodiments, conducting 96 therapy may involve activating a reactor 20 or the contents thereof. Likewise, conducting 96 a therapy session may involve proper application of the distributor 12 to the person of the user such as by adhering an adhesive strip 42 to the skin of a user in order to position the output ports 14 in the nostrils of a user for receiving nitric oxide therefrom. It may include assembling the necessary conduit 18 or line 18 with the distributor 12 to send nitric oxide from the reactor 20 to the distributor 12, and ultimately to a user.

Monitoring may involve adding gauges or meters, taking samples, or the like in order to verify that the delivery of nitric oxide from the reactor 20 to the distributor 12 does meet the therapeutically designed maximum and minimum threshold requirements specified by a medical professional.

Ultimately, after the expiration of an appropriate time specified, or the exhaustion of a content of a reactor 20, a therapy session may be considered completed. Accordingly, the apparatus 10 may be removed 98 from use, discarded, or the like. Accordingly, the removal or discarding 98 of the apparatus 10 may be by parts, or by the entirety. For example, the distributor 12, if it does not include an integrated reactor therewithin, may simply act as a manifold and be reused with a new reactor 20.

It is contemplated that the reactor 20 may typically be a single dose reactor but need not be limited to such. Multiple-dose or reusable reactors may also be used. For example, the reactor 20 may actually contain a cartridge placed within the wall 56. The internal structure of the cartridge may be ruptured in the appropriate seal locations, such as the seals 64, 70 by a mechanism associated with the main containment vessel or wall 56, and thus activated. Accordingly, the reactor 20 may be reused by simply replacing the cartridge of materials containing the reactant volumes 66, 68.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

According to some embodiments, an anti-microbial gas apparatus and method is provided. An apparatus and method administering nitric oxide at very high concentrations to healthy skin, tools, implements, support surfaces, and sterile fields to provide sterilization. The apparatus and method providing sterilization in a dry environment lacking the common undesirable effects of anti-microbial soaps and antiseptics. This invention relates to anti-microbial materials, processes, and equipment, and more particularly to novel systems and methods for employing nitric oxide gas as a sterilizing agent.

Hospitals have a sterilization problem. Documented evidence shows that not everyone washes regularly nor washes effectively. As a result, staph infections still abound.

Nitric oxide (NO) is the subject of Nobel Prize-winning work. The significance of nitric oxide as a vascular relaxing factor is well established. Likewise, it appears that nitric oxide has a topical ability to trigger a reduction of inflammation. For example, nitric oxide has some ability to inhibit those factors responsible for engaging the inflammation response of the body.

Meanwhile, drug-resistant staph infections, antibiotic-resistant strains of bacteria, and the like have become a great concern for the modern medical community. Antibacterial soaps are washed into sewer systems, damaging colonies of useful bacteria as well as fostering resistance in undesirable bacteria. Accordingly, some express a concern that with such ubiquitous use of antibacterial compositions, desirable bacteria will decline in the environment while antibiotic-resistant strains of undesirable bacteria will thrive to displace them in the environment.

Likewise, equipment often requires preparation of liquid sterilization. Chemicals such as alcohol and other antiseptic preparations have environmental effects that may be undesirable, particularly in the long term. Meanwhile, metal instruments can be sterilized by heat in an autoclave. Nevertheless, many instruments now have disposable (i.e., low melting point) plastic handles with metal working surfaces.

An inexpensive process is needed that does not require the heat of an autoclave. What is needed is a material, method, and apparatus for sterilizing or purifying surfaces on instruments as well as skin surfaces of persons. Persons cannot tolerate the temperatures and isolation required for autoclaving instruments. Meanwhile, inexpensive instruments do not tolerate temperature either. What is needed is a manner, material, and system for destroying microbes on the skin of a user, and on surfaces of instruments and other tools used in medical facilities.

In view of the foregoing, in one aspect of an apparatus and method in accordance with the invention, nitric oxide gas may be introduced into an enclosed environment in comparatively extremely high concentrations. Inhaling nitric oxide is a therapy requiring careful monitoring and comparatively low doses to be effective without being toxic. However, healthy skin may be introduced to very high doses over 500 parts per million. Likewise, in one embodiment of an apparatus and method in accordance with the invention, inanimate objects such as surgical tools, other implements, sterile fields, and the like may be exposed to substantially any very high concentration of nitric oxide. The concentration may be applied for sufficient time for the nitric oxide to kill any microbes.

Typically, the transport processes affecting free and forced convection of gases are very much slower than those of liquids. For example, heat transfer, diffusion transport, and the like, whether in free or forced convection, operate more effectively in liquids. For example, scrubbing healthy skin with an anti-microbial liquid will quickly expose the entire surface of the skin to the active ingredient. By contrast, gasses are much less dense, move more slowly, and provide less transport capacity for chemical species, heat, and the like.

Nevertheless, it has been found that creating an enclosed environment to contain nitric oxide, while exposing a material or surface to nitric oxide is very effective. Displacing oxygen, nitric oxide will not support life. Moreover, being somewhat chemically unstable, nitric oxide readily reacts with oxygen. Accordingly, nitric oxide will strip out any oxygen present. Likewise, by being reactive, nitric oxide operates as a chemical radical, scavenging chemicals and thus attacking microbes.

It has been found that an enclosed environment having introduced thereto a flux of nitric oxide, and a flush port for exit thereof can maintain substantially a constant concentration of nitric oxide exposed to the surface all enclosed within the enclosed nitric oxide environment.

It is contemplated that certain embodiments of an apparatus and method in accordance with the invention may rely on concentration gradients to drive diffusion of nitric oxide to contact, engage, and neutralize microbes. Accordingly, it is contemplated that within reason, concentration gradients may be increased in inverse proportion to exposure times. Experiments by applicant have shown substantial reductions in colony counts of bacteria exposed to nitric oxide. According to Fick's law of diffusion, a rate of diffusion is directly proportional to concentration gradients of a material being diffused. Accordingly, the experiments have demonstrated the efficacy of nitric oxide as a sterilizing agent against microbes on healthy skin.

In a direct comparison between scrubbing with antibacterial soaps compared to immersing in a substantially enclosed environment containing exclusively nitric oxide diluted with ambient air, the anti-microbial effects of nitric oxide have been shown to be superior to soaps. Moreover, once released into the atmosphere, nitric oxide may react to more various oxides of nitrogen without long term adverse effects in medically-significant quantities. The invention contemplates that concentrations of from about 500 parts per million up to 1,000,000 parts per million of nitric oxide, substantially pure nitric oxide, may be used to provide sterilization and other microbial effects on healthy skin, surgical instruments, sterile fields, support surfaces, and the like.

Forced convection may be increased in order to increase the exposure concentration and decrease the time required for nitric oxide to contact and sterilize surfaces. According to the transport processes controlled by Fick's law of diffusion, a 15-minute exposure to 1,000 parts per million may be scaled to a 1.5-minute exposure at 10,000 parts per million. Any non-linearaties of scaling may be accommodated by increasing times and increasing the vigor of forced convection flows exposing a surface to nitric oxide.

The invention advances the art in several respects. For example, nitric oxide in accordance with the invention may be applied to healthy tissue, not relying on vascular dilation, and not relying on de-activating the inflammation triggers. Rather, nitric oxide in accordance with the invention may be applied to decontaminate, sterilize, or otherwise destroy microbes directly. Accordingly, very short periods of time may be used at very high concentrations. Exposure times may be as low as five minutes or less. In some embodiments, exposure times of less than one minute may provide substantially complete sterilization of equipment or healthy skin. Exposure times on the order of seconds may rely on nitric oxide moving in forced convection over a surface enclosed in an environment containing a preselected concentration of nitric oxide.

The exposure of healthy tissues or equipment to a single dose of nitric oxide can provide sterilization in accordance with the invention. Meanwhile, the cost of nitric oxide provided by a generator is substantially less expensive on the order of less than one percent of the cost of conventional nitric oxide delivery.

Rather than operating as a drug delivery protocol, a method in accordance with the present invention may operate as a poisoning of microbes. Rather than treating a disease through multiple applications of a drug during multiple weeks of therapy a single dose may provide adequate antisepsis. In one method in accordance with the invention, a single exposure sterilizes a surface, whether a surface of an implement, a supporting surface, a sterile field, or healthy tissues of a subject. A method in accordance with the invention provides an anti-microbial effect in a single exposure sufficiently effective to replace conventional scrubbing with liquid, anti-microbial compositions. By relying on an enclosed environment, concentrations may be controlled. Otherwise, chemical activity as well as uncontrolled dilution may negatively effect the concentration of nitric oxide.

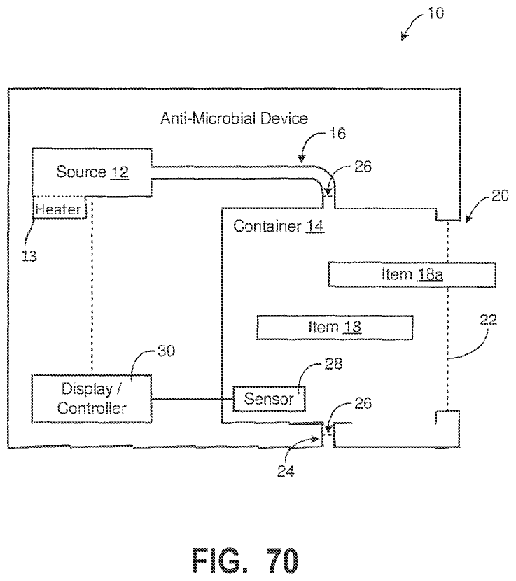

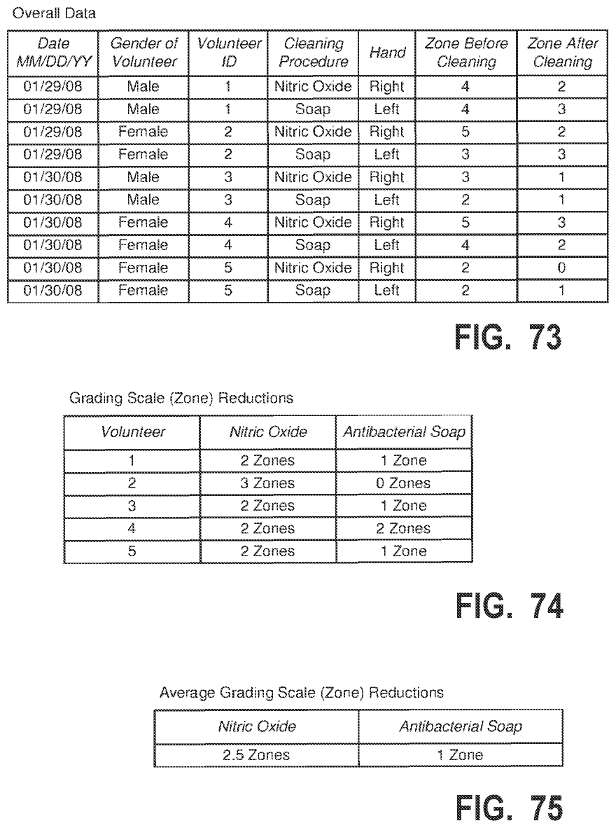

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which FIGS. 70-75 disclose additional advantageous aspects and features.

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings.

Referring to FIG. 70, an apparatus 10 or anti-microbial device 10 in accordance with the present invention may include a source 12 of nitric oxide. A source 12 may be any suitable mechanism for delivering nitric oxide. In selected embodiments, a source 12 may be a tank of nitric oxide. In other embodiments, a source 12 may be a nitric oxide generator. For example, a source 12 may be any of the nitric oxide generators disclosed in U.S. Pat. No. 7,220,393 issued May 22, 2007, U.S. patent application Ser. No. 11/751,523 filed May 21, 2007, U.S. patent application Ser. No. 12/361,123 filed Jan. 28, 2009, U.S. patent application Ser. No. 12/361,151 filed Jan. 28, 2009, U.S. Patent Application Ser. No. 61/025,226 filed Jan. 31, 2008, U.S. Patent Application Ser. No. 61/025,230 filed Jan. 31, 2008, and U.S. Patent Application Ser. No. 61/043,064 filed Apr. 7, 2008, each of which is hereby incorporated by reference.

A source 12 may include a heat source, or heater 13. The heater 13 may be used to heat the contents of the source 12, including without limitation, heating a nitrate and a nitrate in the presence of a metal to produce the desired nitric oxide. The heater 13 may be of any suitable type that can apply heat to the source 12 in a safe, effective manner, including without limitation, heaters that utilize a chemical reaction and may be contained within the apparatus 10, heaters that utilize a fuel that is combusted and may ne contained within the apparatus 10, and heater that utilize electricity and may be contained within the apparatus 10 or may require a connection outside the apparatus 10.

A source 12 may be connected to a container 14 by a conduit 16. The conduit 16 may conduct nitric oxide from the source 12 to the container 14. A container 14 may be any mechanism suitable for maintaining a nitric oxide environment over or around items 18 or surfaces of items 18. A container 14 may be formed of flexible materials, rigid materials, elastic materials or the like. A container 14 may comprise a bag, box, dome or hemisphere, glove, or the like.

Items 18 may be introduced within a container 14 in any suitable manner. Items 18 may be processed through a container 14 in batches. Alternatively, items 18 may pass through a container 14 on a conveyor system. Accordingly, an anti-microbial device 10 in accordance with the present invention may be part of a continuous manufacturing process.

A container 14 in accordance with the present invention may include an opening 20 for introducing items into the container 14 or for exposing the contents of a container 14 to a surface. In selected embodiments, when the apparatus 10 is in use, the opening 20 may be blocked or sealed. For example, a barrier 22 such as a door 22 may close to seal the opening 20. In other embodiments, an item 18 a to be sterilized may extend from the interior of the container 14 to the exterior of the container 14. In such embodiments, a barrier 22 may provide a seal between the container 14 and the item 18 a.

For example, in certain embodiments, an apparatus 10 in accordance with the present invention may be configured to sterilize the hands of a surgeon. In one such embodiment, the container 14 may be a bag and the barrier 22 may be tape sealing the bag against the arm of the surgeon. In other such embodiments, the container 14 may be substantially rigid e. (g., a box) and the barrier 22 may be an elastic or inflatable structure that seals against the arm or arms of the surgeon. Thus, a barrier 22 in accordance with the present invention may be adapted according to the intended use of the container 14.

In selected embodiments, a container 14 may include a vent 24 or exhaust port 24. A vent 24 may permit additional nitric oxide to be delivered to the container 14, without increasing the pressure within the container 14. Accordingly, a vent 24 may assist in maintaining a desired concentration of nitric oxide within a container 14.

A vent 24 may include a check valve 26 ensuring that only outgoing flows pass therethrough. If desired or necessary, the conduit 16 may also include a check valve 26. A check valve 26 in the conduit 16 may ensure that only flows from the source 12 to the container 14 may pass through the conduit 14.

An apparatus 10 in accordance with the present invention may include a sensor 28 for monitoring the concentration of nitric oxide within, or delivered to, a container 14. In selected embodiments, a sensor 28 may be connected to a display 30. Accordingly, a user or technician may monitor the concentration of nitric oxide and make adjustments (e.g., to the source 12) as necessary.

Alternatively, a sensor 28 may be connected to a computerized controller 30. Accordingly, a controller 30 may perform certain tasks based on the information received from the sensor 30. For example, a controller 30 may make adjustments as necessary to maintain the desired concentration of nitric oxide within the container 14, controlling the ratio of a stream of nitric oxide to a flow of ambient air. Additionally, a controller 30 may monitor how long the apparatus 10 has been in use and advise a user or technician when a particular sterilization cycle is complete.

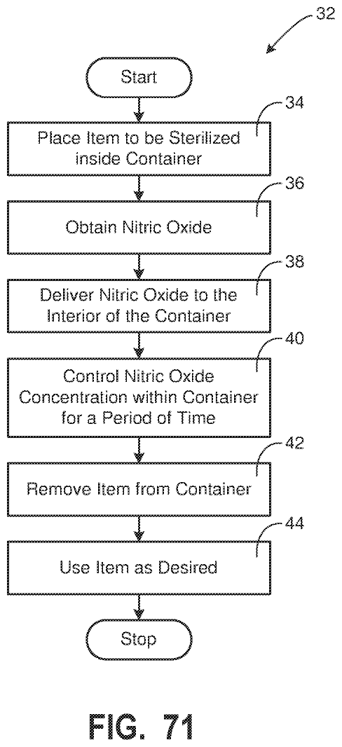

Referring to FIG. 71, a method 32 in accordance with the present invention may begin with placing 34 an item 18 to be sterilized within, or at least in fluid contact with the contents of, the container 14. Once nitric oxide has been obtained 36, it may be introduced 38 into the container 14. The concentration of nitric oxide within the container 14 may be controlled 40 for a period of time. The concentration and time may be selected to ensure that proper sterilization has been achieved. Once the sterilization cycle is complete, the item 18 may be removed 42 from the container 14 and used 44 as desired.

### EXAMPLE

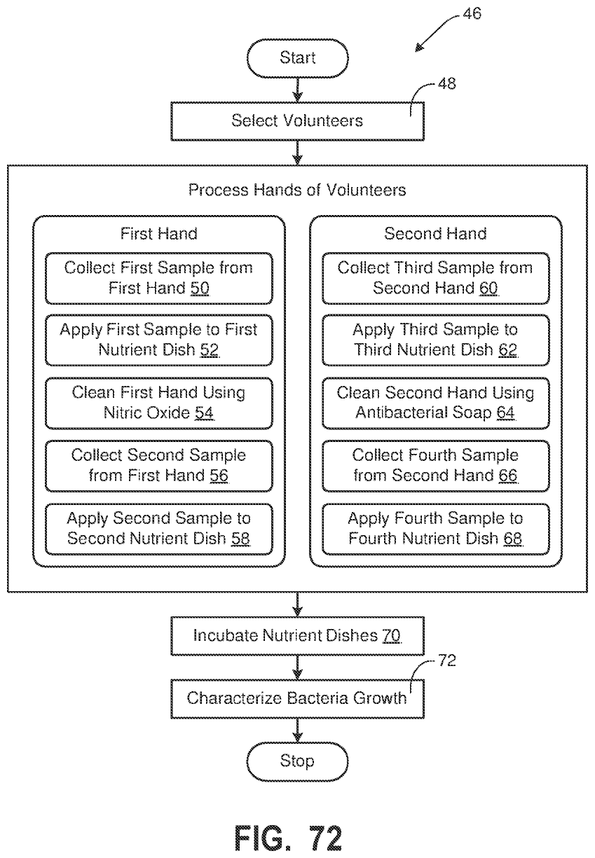

An experiment 46 used to determine the anti-microbial effectiveness of nitric oxide is illustrated in FIG. 72. In the experiment, five volunteers were selected 48. From a first hand of each volunteer, a technician using sterile gloves collected 50 a sample. This was accomplished by rubbing the back of the volunteer's hand with a sterile cotton collection swab for ten seconds. The swab was then applied 52 to a nutrient agar petri dish using the five corner or zone dilution method.

The five corner or zone dilution method involves mechanically diluting bacteria on a streak (blood agar) plate by sequentially spreading the bacteria across the plate in each of five zones. As the concentration of bacteria increases so do the number of zones containing bacteria. Bacteria on agar plate become visible as distinct circular colonies. Each colony represents an individual cell which has divided repeatedly to form a patch. The number of bacteria can be estimated by counting the number of patches or how far the bacteria is diluted by streaking it on the agar plate through the five zones.

After the sample was collected 50, the first hand was cleaned 54 using nitric oxide. This was done by placing the hand of the volunteer into a one-gallon plastic freezer bag. The bag was then inflated with nitric oxide through tubing attached to a portable nitric oxide generator. The open end of the bag was taped closed against the volunteer's forearm. A nitric oxide monitor assisted in keeping the nitric oxide concentration within the bag at 1,000 parts-per-million (ppm).

The volunteer maintained the hand inside the bag for fifteen minutes. After the fifteen minutes, the hand was removed from the bag in a sterile manner (i.e., the hand was not permitted to contact any non-sterile objects). Using sterile gloves and a sterile cotton collection swab, the technician collected 56 a second sample by rubbing the swab on the back of the hand for ten seconds. The swab was then applied 58 to a nutrient dish as explained above.

A similar process was followed with the volunteer's other hand. A technician using sterile gloves collected 60 a sample. This was accomplished by rubbing the back of the volunteer's hand with a sterile cotton collection swab for ten seconds. The swab was then applied 62 to a nutrient agar petri dish using the five corner or zone dilution method.

The second hand was then cleaned 64 using DIAL antibacterial soap. This cleaning lasted two minutes and was accomplished using the volunteers convention hand wasting techniques. After the second hand was cleaned 64, the technician used sterile gloves and a sterile cotton collection swab to collect 66 a sample by rubbing the swab on the back of the hand for ten seconds. The swab was then applied 68 to a nutrient dish as explained above.

The nutrient dishes were then incubated at thirty-five degrees Celsius for forty-eight hours. Using a zone-based grading scale for bacterial colonization, the technician then graded 72 the dishes for each volunteer. On this scale, bacteria growth extending no further than zone 1 was characterized as “zone 1,” bacteria growth extending no further than zone 2 was characterized “zone 2,” etc. Accordingly, the higher the zone number, the greater the number of bacteria.

The data collected from the experiment is present in FIGS. 73-75. From the data, it can be seen that hands exposed to 1,000 ppm of nitric oxide for fifteen minutes had a lower bacterial colony count than hands washed with DIAL antibacterial soap for 2 minutes.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

1-9. (canceled)

10. A system for providing a topical nitric oxide therapy, comprising:
a nitrite medium in a first container, the nitrite medium comprising about 3% of a nitrite component by weight, wherein the nitrite component is sodium nitrite or potassium nitrite wherein the nitrite medium comprises greater than about 1% hydroxypropyl methylcellulose polymers by weight;
an acidic medium in a second container, the acidic medium comprising about 9% by weight of one or more acidic reactants, wherein the acidic reactant is selected from the group consisting of citric acid, lactic acid, ascorbic acid, acetic acid, and a combination thereof; and
wherein the nitrite medium and the acidic medium are configured to be combined to form a nitric oxide topical medium producing nitric oxide suitable for topical application and suitable for administering nitric oxide therapy wherein a therapeutically effective amount of the nitric oxide topical medium is applied to a treatment surface suitable for receiving nitric oxide therapy, whereby the application of the therapeutically effective amount is adapted to deliver a dose of nitric oxide at the treatment surface of a patient, wherein the treatment surface of a patient is the patient's skin.

11. (canceled)

12. The system of claim 10, wherein one or more of the first and second containers are configured to dispense a medium by one or more of a pump action, a squeezing action, and a shaking action.

13. The system of claim 10, wherein the therapeutically effective amount of the topical medium is configured to produce nitric oxide gas having a concentration of between about 500 ppm and about 1000 ppm.

14. The system of claim 10, wherein the therapeutically effective amount of the topical medium is configured to produce nitric oxide gas having a concentration of between about 1000 ppm and about 2000 ppm.

15. The system of claim 10, wherein the dose of nitric oxide provides a localized vasodilation treatment to the patient.

16. The system of claim 10, wherein the dose of nitric oxide provides a systemic vasodilation treatment to the patient.

17. A composition system comprising a combination of a nitrite medium and an acidic medium for production of a topical medium for topical application of nitric oxide therapy, the composition system comprising:
a nitrite medium in a first container, the nitrite medium comprising a nitrite component, wherein the nitrite component is sodium nitrite or potassium nitrite, wherein the nitrite medium comprises greater than about 1% hydroxypropyl methylcellulose polymers by weight;
an acidic medium in a second container, the acidic medium comprising an acidic component, wherein the acidic component is selected from the group consisting of citric acid, lactic acid, ascorbic acid, acetic acid, and a combination thereof; and
wherein the nitrite medium and the acidic medium are configured to be combined to form a nitric oxide topical medium producing nitric oxide suitable for topical application and suitable for administering nitric oxide therapy wherein a therapeutically effective amount of the nitric oxide topical medium is applied to a treatment surface suitable for receiving nitric oxide therapy, whereby the application of the therapeutically effective amount is adapted to deliver a dose of nitric oxide at the treatment surface of a patient, wherein the treatment surface of a patient is the patient's skin, wherein the treatment surface of a patient is the patient's skin.

18. The composition system of claim 17, wherein the nitrite medium is a nitrite gel medium and the acidic medium is an acidic gel medium.

19. The composition system of claim 17, wherein the nitrite medium is a nitrite lotion medium and the acidic medium is an acidic lotion medium.

20. The composition system of claim 17, wherein the nitrite medium is a nitrite gel medium and the acidic medium is an acidic lotion medium.

21. The composition system of claim 17, wherein the nitrite medium is a nitrite lotion medium and the acidic medium is an acidic gel medium.

22. The composition system of claim 17, wherein the dose of nitric oxide has a concentration of nitric oxide of at least approximately 320 ppm of nitric oxide.

23. The composition system of claim 17, wherein the nitric oxide topical medium comprises approximately 3 grams of the nitrite medium and approximately 3 grams of the acidic medium thereby providing a concentration of nitric oxide of at least approximately 1000 ppm for topical application.

24. The composition system of claim 17, wherein the nitric oxide topical medium is a gel and comprises approximately 3 grams of the nitrite medium and approximately 3 grams of the acidic medium thereby providing a concentration of nitic oxide of at least approximately 840 ppm for topical application.

25. The composition system of claim 17, wherein the nitric oxide topical medium is a lotion and comprises approximately 3 grams of the nitrite medium and approximately 3 grams of the acidic medium thereby providing a concentration of nitic oxide of at least approximately 450 ppm for topical application.

26. (canceled)

27. The composition system of claim 17, wherein the nitrite medium comprises less than about 1% by weight of a combination of additives comprising two or more of methylchloroisothiazolinone, methylisothiazolinone, sodium hydroxide, ethylenediamine tetraacetate tetrasodium salt, and citric acid.

28. The composition system of claim 17, wherein the acidic medium comprises greater than about 1% hydroxypropyl methylcellulose polymers by weight.

29. The composition system of claim 17, wherein the acidic medium comprises less than about 1% by weight of a combination of additives comprising two or more of methylchloroisothiazolinone, methylisothiazolinone, sodium hydroxide, and ethylenediamine tetraacetate tetrasodium salt.
